SEQUENCE LISTING (67 sequences)

SEQ ID NO: 1 - RNA, 19 nt, Homo sapiens
  source 1..19: mol_type = other RNA
aatggatttttggagcagg

SEQ ID NO: 2 - RNA, 22 nt, Homo sapiens
  source 1..22: mol_type = other RNA
tcctgtactgagctgccccgag

SEQ ID NO: 3 - RNA, 22 nt, Homo sapiens
  source 1..22: mol_type = other RNA
ttcagatcccagcggtgcctct

SEQ ID NO: 4 - RNA, 17 nt, Homo sapiens
  source 1..17: mol_type = other RNA
ggggcgcggccggatcg

SEQ ID NO: 5 - RNA, 19 nt, Homo sapiens
  source 1..19: mol_type = other RNA
cggggtcggcggcgacgtg

SEQ ID NO: 6 - RNA, 22 nt, Homo sapiens
  source 1..22: mol_type = other RNA
gtgaaatgtttaggaccactag

SEQ ID NO: 7 - RNA, 22 nt, Homo sapiens
  source 1..22: mol_type = other RNA
gctaaggaagtcctgtgctcag

SEQ ID NO: 8 - RNA, 22 nt, Homo sapiens
  source 1..22: mol_type = other RNA
aaaccgttaccattactgagtt

SEQ ID NO: 9 - RNA, 22 nt, Homo sapiens
  source 1..22: mol_type = other RNA
tattgcacttgtcccggcctgt

SEQ ID NO: 10 - RNA, 22 nt, Homo sapiens
  source 1..22: mol_type = other RNA
tcaggctcagtcccctcccgat

SEQ ID NO: 11 - RNA, 23 nt, Homo sapiens
  source 1..23: mol_type = other RNA
caaagtgctgttcgtgcaggtag

SEQ ID NO: 12 - RNA, 18 nt, Homo sapiens
  source 1..18: mol_type = other RNA
gaaaaagtcatggaggcc

SEQ ID NO: 13 - RNA, 22 nt, Homo sapiens
  source 1..22: mol_type = other RNA
cgcatcccctagggcattggtg

SEQ ID NO: 14 - RNA, 22 nt, Homo sapiens
  source 1..22: mol_type = other RNA
ttcaagtaatccaggataggct

SEQ ID NO: 15 - RNA, 23 nt, Homo sapiens
  source 1..23: mol_type = other RNA
tgagaactgaattccataggctg

SEQ ID NO: 16 - RNA, 22 nt, Homo sapiens
  source 1..22: mol_type = other RNA
ctatacgacctgctgcctttct

SEQ ID NO: 17 - RNA, 23 nt, Homo sapiens
  source 1..23: mol_type = other RNA
aatgacacgatcactcccgttga

SEQ ID NO: 18 - RNA, 23 nt, Homo sapiens
  source 1..23: mol_type = other RNA
cccagtgttcagactacctgttc

SEQ ID NO: 19 - RNA, 22 nt, Homo sapiens
  source 1..22: mol_type = other RNA
gagcttattcataaaagtgcag

SEQ ID NO: 20 - RNA, 22 nt, Homo sapiens
  source 1..22: mol_type = other RNA
tcgtaccgtgagtaataatgcg

SEQ ID NO: 21 - RNA, 23 nt, Homo sapiens
  source 1..23: mol_type = other RNA
taccctgtagaaccgaatttgtg

SEQ ID NO: 22 - RNA, 21 nt, Homo sapiens
  source 1..21: mol_type = other RNA
agccgcggggatcgccgaggg

SEQ ID NO: 23 - RNA, 20 nt, Homo sapiens
  source 1..20: mol_type = other RNA
tatggaggtctctgtctggc

SEQ ID NO: 24 - RNA, 22 nt, Homo sapiens
  source 1..22: mol_type = other RNA
tagcagcacgtaaatattggcg

SEQ ID NO: 25 - RNA, 23 nt, Homo sapiens
  source 1..23: mol_type = other RNA
tgtgcaaatccatgcaaaactga

SEQ ID NO: 26 - RNA, 21 nt, Homo sapiens
  source 1..21: mol_type = other RNA
atcacattgccagggatttcc

SEQ ID NO: 27 - RNA, 23 nt, Homo sapiens
  source 1..23: mol_type = other RNA
taaagtgcttatagtgcaggtag

SEQ ID NO: 28 - RNA, 22 nt, Homo sapiens
  source 1..22: mol_type = other RNA
tagcttatcagactgatgttga

SEQ ID NO: 29 - RNA, 22 nt, Homo sapiens
  source 1..22: mol_type = other RNA
tgaggtagtaggttgtatagtt

SEQ ID NO: 30 - RNA, 22 nt, Homo sapiens
  source 1..22: mol_type = other RNA
tccgagcctgggtctccctctt

SEQ ID NO: 31 - RNA, 24 nt, Homo sapiens
  source 1..24: mol_type = other RNA
ttaatgctaatcgtgataggggtt

SEQ ID NO: 32 - RNA, 21 nt, Homo sapiens
  source 1..21: mol_type = other RNA
caaaacgtgaggcgctgctat

SEQ ID NO: 33 - RNA, 23 nt, Homo sapiens
  source 1..23: mol_type = other RNA
taccctgtagatccgaatttgtg

SEQ ID NO: 34 - RNA, 22 nt, Homo sapiens
  source 1..22: mol_type = other RNA
tggagtgtgacaatggtgtttg

SEQ ID NO: 35 - RNA, 21 nt, Homo sapiens
  source 1..21: mol_type = other RNA
tcacagtgaaccggtctcttt

SEQ ID NO: 36 - RNA, 22 nt, Homo sapiens
  source 1..22: mol_type = other RNA
actcggcgtggcgtcggtcgtg

SEQ ID NO: 37 - RNA, 22 nt, Homo sapiens
  source 1..22: mol_type = other RNA
tgagaactgaattccatgggtt

SEQ ID NO: 38 - RNA, 22 nt, Homo sapiens
  source 1..22: mol_type = other RNA
tggacggagaactgataagggt

SEQ ID NO: 39 - RNA, 22 nt, Homo sapiens
  source 1..22: mol_type = other RNA
caaagaattctccttttgggct

SEQ ID NO: 40 - RNA, 22 nt, Homo sapiens
  source 1..22: mol_type = other RNA
aagctgccagttgaagaactgt

SEQ ID NO: 41 - RNA, 21 nt, Homo sapiens
  source 1..21: mol_type = other RNA
agctacatctggctactgggt

SEQ ID NO: 42 - RNA, 22 nt, Homo sapiens
  source 1..22: mol_type = other RNA
cgtgtatttgacaagctgagtt

SEQ ID NO: 43 - RNA, 21 nt, Homo sapiens
  source 1..21: mol_type = other RNA
ttcacagtggctaagttccgc

SEQ ID NO: 44 - RNA, 22 nt, Homo sapiens
  source 1..22: mol_type = other RNA
aaaagctgggttgagagggcga

SEQ ID NO: 45 - RNA, 22 nt, Homo sapiens
  source 1..22: mol_type = other RNA
ctggccctctctgcccttccgt

SEQ ID NO: 46 - RNA, 23 nt, Homo sapiens
  source 1..23: mol_type = other RNA
gcaaagcacacggcctgcagaga

SEQ ID NO: 47 - RNA, 22 nt, Homo sapiens
  source 1..22: mol_type = other RNA
aattgcacggtatccatctgta

SEQ ID NO: 48 - RNA, 23 nt, Homo sapiens
  source 1..23: mol_type = other RNA
agctcggtctgaggcccctcagt

SEQ ID NO: 49 - RNA, 21 nt, Homo sapiens
  source 1..21: mol_type = other RNA
caggagtggggggtgggacgt

SEQ ID NO: 50 - RNA, 22 nt, Homo sapiens
  source 1..22: mol_type = other RNA
tgcggggctagggctaacagca

SEQ ID NO: 51 - RNA, 22 nt, Homo sapiens
  source 1..22: mol_type = other RNA
aacccgtagatccgatcttgtg

SEQ ID NO: 52-65 - skipped (14 entries)

SEQ ID NO: 66 - RNA, 102 nt, Homo sapiens
  source 1..102: mol_type = other RNA
ggtcctctgactctcttcggtgacgggtattcttgggtggataatacggattacgttgttattgcttaagaatacgcgtagtcgaggagagtaccagcggca

SEQ ID NO: 67 - RNA, 23 nt, Homo sapiens
  source 1..23: mol_type = other RNA
ttattgcttaagaatacgcgtag

(19) United States
(12) Patent Application Publication
(10) Pub. No.: US 2026/0201469 A1
(43) Pub. Date: Jul. 16, 2026

(54) EXTRACELLULAR VESICLES, ISOLATION AND USE AS BIOMARKERS

(71) Applicant: THE UNIVERSITY OF NEWCASTLE, New South Wales (AU)

(72) Inventors: Murray John CAIRNS, New South Wales (AU); Michelle Mia BARNETT, New South Wales (AU)

(21) Appl. No.: 19/129,467

(22) PCT Filed: Nov. 14, 2023
(86) PCT No.: PCT/AU2023/051154

§ 371 Date: May 13, 2025

(30) Foreign Application Priority Data
Nov. 14, 2022 (AU) 2022903418

(51) Int. Cl. C12Q 1/6883; G01N 1/30

(52) U.S. Cl. CPC C12Q 1/6883; G01N 1/30; C12Q 2600/158; C12Q 2600/178

(57) ABSTRACT

The present disclosure relates to neuron derived extracellular vesicles and methods for their isolation as well as their use as biomarkers, including for neurodegenerative and/or neuropsychiatric diseases. The present disclosure also relates to identification of small non-coding RNA and their use for diagnosis and/or prognosis and/or therapy stratification.

### TECHNICAL FIELD

The present disclosure relates to neuron derived extracellular vesicles, methods of isolation of neuron derived extracellular vesicles and use as biomarkers. The present disclosure also relates to identification of small non-coding RNA and their use for the diagnosis and/or prognosis and/or therapy stratification of a disease or condition including neurodegenerative diseases and/or neuropsychiatric diseases.

### BACKGROUND OF INVENTION

Central nervous system disorders are traditionally dichotomized between early-onset neurodevelopmental and late-onset neurodegenerative diseases.

Neurodegeneration is a common underlying pathomechanism in a wide range of neurologic diseases and psychiatric disorders which develop through polygenic interaction, multifactorial causative factors, and heterogenous pathogenesis. The neurologic diseases which manifest the neurodegeneration include Alzheimer's diseases, Parkinson's diseases, multiple sclerosis, amyotrophic lateral sclerosis, Huntington's disease, epilepsy, etc. while psychiatric disorders include depressive disorder, substance abuse disorder, anxiety disorder, post-traumatic stress disorder, bipolar disorder, schizophrenia, somatic symptom disorder, and autism spectrum disorder.

These incurable and debilitating diseases affect millions of people worldwide, and therefore represent a major global health challenge with severe implications for individuals and society.

Among current diagnostics for neurodegeneration, neuropathology is considered as the gold standard. However, it is usually based on an autopsy that is done after the death of a patient. Therefore, there is a need for an effective non-invasive diagnostic method that can be employed for an early detection of neurodegeneration when a pharmacological intervention is still possible.

The Diagnostic and Statistical Manual (DSM) is the handbook widely used by clinicians and psychiatrists to diagnose psychiatric disorders. But such diagnosis lacks objective measures of psychopathology and biomarkers that reliably delineate normal from disease states, and one disease state from another. Moreover, more so than in any other area of medicine, conceptions about mental health and disease remain profoundly influenced by social and cultural norms and stigma. Therefore, there is a need for a diagnostic method useful in prevention, diagnosis, drug response or drug development in psychiatric disorders.

### SUMMARY OF INVENTION

The present inventors determined miRNA expression in microtubule associated protein (MAP) enriched extracellular vesicles from a large cohort of individuals with schizophrenia and nonpsychiatric controls. The present inventors observed significant dysregulation of miRNA in schizophrenia subjects, particularly those with severe cognitive deficits which displayed a much larger magnitude of miRNA dysregulation than was observed in their cognitively spared counterparts. The predicted target gene interactions of cognitive deficit associated miRNA were enriched at the synapse, the process of neurogenesis and are overrepresented in neurotransmitter receptors and postsynaptic signal transmission pathways. These data show that miRNA from extracellular vesicles, particularly, extracellular vesicles enriched for neuronal origin are altered in individuals with schizophrenia and reflect a broader dysregulation in the posttranscriptional regulatory environment of the synapse.

The data suggests that the dysregulated miRNA observed are contributing to perturbations of neurogenesis and synaptic plasticity, possibly via signalling pathways important for neuroprotection. This methodology has broad applications for the diagnosis of diseases that affect tissues that are difficult to biopsy, particularly those affecting the CNS.

Accordingly, the present inventors have surprisingly developed a method of enriching for neuron derived extracellular vesicles which can be used in diagnosis and/or prognosis of a disease or condition (including but not limited to neurodegenerative diseases and/or neuropsychiatric diseases), monitoring the effectiveness of a treatment and treatment response, and for use in patient stratification.

Accordingly, the present disclosure provides a method of diagnosing a disease state of a subject, said method comprising the steps of:
a) measuring the total circulating neuronal origin extracellular vesicle small non-coding RNA level, in a sample from the subject; and
b) comparing the level to a reference level of total circulating neuronal origin extracellular vesicle small non-coding RNA level, wherein a decreased level of the total circulating neuronal origin extracellular vesicle non-coding RNA, compared to the reference level is indicative of a disease state or a stage of development of a disease state.

In one embodiment, the reference level of the total circulating neuronal origin extracellular vesicle small non-coding RNA is the level of the total circulating neuronal origin extracellular vesicle small non-coding RNA in a sample from a normal subject.

In one or a further embodiment, the reference level of the total circulating neuronal origin extracellular vesicle small non-coding RNA is a threshold level, for example, a cut-off value.

In one or a further embodiment, the small non-coding RNA is microRNA (miRNA).

In one or a further embodiment, the sample comprises microvesicles and/or exosomes.

In one or a further embodiment, the sample is a sample enriched for extracellular vesicles, for example, exosomes.

In one or a further embodiment, the sample is enriched for extracellular vesicles of neuronal origin according to a method of the disclosure.

In one or a further embodiment, the sample is blood, serum or plasma.

In one or a further embodiment, the disease is a neurodegenerative disease or neuropsychiatric disease.

In one embodiment, the disease is Schizophrenia.

In one embodiment, a log 10 fold decrease of at least about 0.24, about 0.25, about 0.26, about 0.27, about, 0.28, about 0.29, about 0.3 total circulating neuronal origin extracellular vesicle small non-coding RNA compared to the reference level of total circulating neuronal origin extracellular vesicle small non-coding RNA in a sample from a normal subject is indicative of Schizophrenia or a predisposition to Schizophrenia.

In another embodiment, the disease is Schizophrenia with cognitive deficit.

In one embodiment, a log 10 fold decrease of at least about 0.23, about 0.24, about 0.25, about 0.26, about 0.27, about 0.28, about 0.3 total circulating neuronal origin extracellular vesicle small non-coding RNA compared to a reference level of total circulating neuronal origin extracellular vesicle small non-coding RNA in a sample from a normal subject is indicative of Schizophrenia with cognitive deficit.

In another embodiment, the disease is Schizophrenia with spared condition.

In one embodiment, a log 10 fold decrease of at least about 0.25, about 0.26, about 0.27, about 0.28, about 0.29, about 0.3 total circulating neuronal origin extracellular vesicle small non-coding RNA compared to a reference level of total circulating neuronal origin extracellular vesicle small non-coding RNA in a sample from a normal subject is indicative of Schizophrenia with spared cognition.

The present inventors have also found that identification of one or more small non-coding RNAs in a sample can be used as a diagnostic or prognostic marker of a disease or condition such as, for example, a neurodegenerative disease and/or a neuropsychiatric disease, and can also be used to monitor changes to disease state further to therapeutic intervention.

In some embodiments of the disclosure, the level of one or more small non-coding RNAs in the sample can be used to stage the severity of the disease or condition. For example, the level of one or more small non-coding RNAs in the sample can be used to stage the severity of a neurodegenerative disease and/or a neuropsychiatric disease.

In addition to use as a diagnostic or prognostic tool and for objective monitoring of treatment efficacy in subjects, the one or more small non-coding RNAs can be use in the discovery and development of new treatments of a disease or condition.

Accordingly, the present disclosure also provides a method of diagnosing a disease state of a subject, said method comprising the steps of:
a) measuring the level of one or more small non-coding RNA in a sample from the subject, wherein the one or more small non-coding RNA comprises a nucleotide sequence shown in any one of SEQ ID NOs: 1 to 29, and wherein, the sample comprises extracellular vesicles, for example, extracellular vesicles of neuronal origin; and
b) comparing the level of the one or more small non-coding RNA to a reference level of the one or more small non-coding RNA, wherein an increased or decreased level of the one or more small non-coding RNA compared to the reference level is indicative of a disease state or a stage of development of a disease state.

In one embodiment, the reference level of the one or more small non-coding RNA is the level of the one or more small non-coding RNA in a sample from a normal subject.

In one or a further embodiment, the reference level of the one or more small non-coding RNA is a threshold level, for example, a cut-off value.

In one or a further embodiment, the one or more small non-coding RNA is a microRNA (miRNA).

In one or a further embodiment, the extracellular vesicles in the sample comprise microvesicles and/or exosomes, for example, microvesicles and/or exosomes of neuronal origin.

In one or a further embodiment, the sample comprising extracellular vesicles is a sample enriched for extracellular vesicles, for example, exosomes.

In one or a further embodiment, the sample comprising extracellular vesicles is enriched for extracellular vesicles of neuronal origin, for example, according to a method of the disclosure.

In one or a further embodiment, the sample is blood, serum or plasma.

In one or a further embodiment, the disease is a neurodegenerative disease or neuropsychiatric disease.

In one embodiment, the disease is Schizophrenia.

In one embodiment, an increased level of a small non-coding RNA comprising the nucleotide sequence shown in SEQ ID NO: 1 compared to the reference level(s) is indicative of Schizophrenia or a predisposition to Schizophrenia. For example, a log₂ fold increase of at least about 0.5, about 0.6. about 0.7, about 0.8 compared to a reference level of the small non-coding RNA in a sample from a normal subject is indicative of Schizophrenia or a predisposition to Schizophrenia.

In one or a further embodiment, the magnitude of the differential level of a small non-coding RNA comprising the nucleotide sequence shown in SEQ ID NO: 1 is increased in more severe disease states of Schizophrenia (such as Schizophrenia with cognitive deficit and treatment resistant Schizophrenia), when compared with Schizophrenia as a whole undifferentiated group.

In one or a further embodiment, targets of a small non-coding RNA comprising the nucleotide sequence shown in SEQ ID NO: 1 are specifically enriched in the brain.

In one or a further embodiment, targets of a small non-coding RNA comprising the nucleotide sequence shown in SEQ ID NO: 1 overlap with targets of a small non-coding RNA comprising the nucleotide sequence shown in SEQ ID NO: 67.

In one or a further embodiment, a decreased level of a small non-coding RNA comprising the nucleotide sequence shown in SEQ ID NO: 2 compared to the reference level(s) is indicative of Schizophrenia or a predisposition to Schizophrenia. For example, a log₂ fold decrease of at least about 0.3, about 0.4, about 0.5 compared to a reference level of the small non-coding RNA in a sample from a normal subject is indicative of Schizophrenia or a predisposition to Schizophrenia.

In one embodiment, the disease is Schizophrenia with cognitive deficit.

In one embodiment, an increased level of one or more small non-coding RNA(s) comprising the nucleotide sequence shown in any one of SEQ ID NOs: 1, and 3 to 7 compared to the reference level(s) of the one or more small non-coding RNA(s) is indicative of Schizophrenia with cognitive deficit. For example, a log₂ fold increase of at least about 0.7 to at least about 1.3 compared to a reference level(s) of the one or more small non-coding RNA(s) in a sample from a normal subject is indicative of Schizophrenia with cognitive deficit.

In another embodiment, an increased level of one or more small non-coding RNA(s) comprising the nucleotide sequence shown in any one of SEQ ID NOs: 1, 3, and 5 compared to the reference level(s) of the one or more small non-coding RNA(s) is indicative of Schizophrenia with cognitive deficit. For example, a log₂ fold increase of at least about 1 to at least about 1.3 compared to a reference level(s) of the one or more small non-coding RNA(s) in a sample from a normal subject is indicative of Schizophrenia with cognitive deficit.

In another embodiment, an increased level of small non-coding RNA comprising the nucleotide sequence shown in SEQ ID NO: 1 compared to the reference level of the small non-coding RNA is indicative of Schizophrenia with cognitive deficit. For example, a log₂ fold increase of at least about 1.3 compared to a reference level of the small non-coding RNA in a sample from a normal subject is indicative of Schizophrenia with cognitive deficit.

In another or further embodiment, an increased level of one or more small non-coding RNA(s) comprising the nucleotide sequence shown in any one of SEQ ID NOs: 3, 5, and 7 compared to the reference level(s) of the one or more small non-coding RNA(s) is indicative of Schizophrenia with cognitive deficit. For example, a log₂ fold increase of at least about 0.7 to at least about 1.1 compared to a reference level(s) of the one or more small non-coding RNA(s) in a sample from a normal subject is indicative of Schizophrenia with cognitive deficit.

In another or further embodiment, an increased level of one or more small non-coding RNA(s) comprising the nucleotide sequence shown in any one of SEQ ID NOs: 4 and 6 compared to the reference level(s) of the one or more small non-coding RNA(s) is indicative of Schizophrenia with cognitive deficit. For example, a log₂ fold increase of at least about 0.8 to at least about 0.9 compared to a reference level(s) of the one or more small non-coding RNA(s) in a sample from a normal subject is indicative of Schizophrenia with cognitive deficit.

In some embodiments, a log₂ fold increase of at least about 1, about 1.1, about 1.2, about 1.25, about 1.26, about 1.27, about 1.28, about 1.29, about 1.3 of the small non-coding RNA comprising the nucleotide sequence shown in SEQ ID NO: 1 compared to a reference level of the small non-coding RNA in a sample from a normal subject is indicative of Schizophrenia with cognitive deficit.

In some embodiments, a log₂ fold increase of at least about 0.9, about 1, about 1.1 of the small non-coding RNA comprising the nucleotide sequence shown in SEQ ID NO: 3 compared to a reference level of the small non-coding RNA in a sample from a normal subject is indicative of Schizophrenia with cognitive deficit.

In some embodiments, a log₂ fold increase of at least about 0.85, about 0.9, about 0.91, about 0.92, about 0.93 of the small non-coding RNA comprising the nucleotide sequence shown in SEQ ID NO: 4 compared to a reference level of the small non-coding RNA in a sample from a normal subject is indicative of Schizophrenia with cognitive deficit.

In some embodiments, a log₂ fold increase of at least about 0.9, about 1, about 1.1 of the small non-coding RNA comprising the nucleotide sequence shown in SEQ ID NO: 5 compared to a reference level of the small non-coding RNA in a sample from a normal subject is indicative of Schizophrenia with cognitive deficit.

In some embodiments, a log₂ fold increase of at least about 0.7, about 0.75, about 0.76, about 0.77, about 0.78, about 0.79, about 0.8 of the small non-coding RNA comprising the nucleotide sequence shown in SEQ ID NO: 6 compared to a reference level of the small non-coding RNA in a sample from a normal subject is indicative of Schizophrenia with cognitive deficit.

In some embodiments, a log₂ fold increase of at least about 0.7, about 0.71, about 0.72, about 0.73, about 0.74, about 0.75 of the small non-coding RNA comprising the nucleotide sequence shown in SEQ ID NO: 7 compared to a reference level of the small non-coding RNA in a sample from a normal subject is indicative of Schizophrenia with cognitive deficit.

In one or a further embodiment, a decreased level of one or more small non-coding RNA(s) comprising the nucleotide sequence shown in any one of SEQ ID NOs: 2, and 8 to 11 compared to the reference level(s) of the one or more small non-coding RNA(s) is indicative of Schizophrenia with cognitive deficit. For example, a log₂ fold decrease of at least about 0.4 to at least about 1.3 compared to a reference level(s) of the one or more small non-coding RNA(s) in a sample from a normal subject is indicative of Schizophrenia with cognitive deficit.

In another embodiment, a decreased level of a small non-coding RNA comprising the nucleotide sequence shown in SEQ ID NO: 8 compared to the reference level of the small non-coding RNA is indicative of Schizophrenia with cognitive deficit. For example, a log₂ fold decrease of at least about 1.3 compared to a reference level of the small non-coding RNA in a sample from a normal subject is indicative of Schizophrenia with cognitive deficit.

In another or further embodiment, a decreased level of a small non-coding RNA comprising the nucleotide sequence shown in SEQ ID NO: 2 compared to the reference level of the small non-coding RNA is indicative of Schizophrenia with cognitive deficit. For example, a log₂ fold decrease of at least about 0.7 compared to a reference level of the small non-coding RNA in a sample from a normal subject is indicative of Schizophrenia with cognitive deficit.

In some embodiments, a log₂ fold decrease of at least about 0.7, about 0.71, about 0.72, about 0.73, about 0.74, about 0.75 of the small non-coding RNA comprising the nucleotide sequence shown in SEQ ID NO: 2 compared to a reference level of the small non-coding RNA in a sample from a normal subject is indicative of Schizophrenia with cognitive deficit.

In some embodiments, a log₂ fold decrease of at least about 1, about 1.1, about 1.2, about 1.25, about 1.26, about 1.27, about 1.28, about 1.29, about 1.3 of the small non-coding RNA comprising the nucleotide sequence shown in SEQ ID NO: 8 compared to a reference level of the small non-coding RNA in a sample from a normal subject is indicative of Schizophrenia with cognitive deficit.

In some embodiments, a log₂ fold decrease of at least about 0.50, about 0.51, about 0.52, about 0.54, about 0.55 of the small non-coding RNA comprising the nucleotide sequence shown in SEQ ID NO: 9 compared to a reference level of the small non-coding RNA in a sample from a normal subject is indicative of Schizophrenia with cognitive deficit.

In some embodiments, a log 2 fold decrease of at least about 0.50, about 0.51, about 0.52, about 0.54, about 0.55, about 0.56, about 0.57, about 0.58, about 0.59, about 0.6 of the small non-coding RNA comprising the nucleotide sequence shown in SEQ ID NO: 10 compared to a reference level of the small non-coding RNA in a sample from a normal subject is indicative of Schizophrenia with cognitive deficit.

In some embodiments, a log₂ fold decrease of at least about 0.30, about 0.35, about 0.36, about 0.37, about 0.38, about 0.39, about 0.4 of the small non-coding RNA comprising the nucleotide sequence shown in SEQ ID NO: 11 compared to a reference level of the small non-coding RNA in a sample from a normal subject is indicative of Schizophrenia with cognitive deficit.

In one or a further embodiment, an increased level of one or more small non-coding RNA(s) comprising the nucleotide sequence shown in any one of SEQ ID NOs: 1, 3, 5, and 7 compared to the reference level(s) of the one or more small non-coding RNA(s) is indicative of Schizophrenia with cognitive deficit. For example, a log 2 fold increase of at least about 1.0 to at least about 1.3 compared to a reference level(s) of the small non-coding RNA(s) in a sample from a subject with cognitively spared Schizophrenia is indicative of Schizophrenia with cognitive deficit.

In another embodiment, an increased level of one or more small non-coding RNA(s) comprising the nucleotide sequence shown in any one of SEQ ID NOs: 1, 3, and 5 compared to the reference level(s) of the one or more small non-coding RNA(s) is indicative of Schizophrenia with cognitive deficit. For example, a log₂ fold increase of at least about 1 to at least about 1.3 compared to a reference level(s) of the one or more small non-coding RNA(s) in a sample from a subject with cognitively spared Schizophrenia is indicative of Schizophrenia with cognitive deficit

In another embodiment, an increased level of a small non-coding RNA comprising the nucleotide sequence shown in SEQ ID NO: 1 compared to the reference level of the small non-coding RNA is indicative of Schizophrenia with cognitive deficit. For example, a log₂ fold increase of at least about 1 compared to a reference level of the small non-coding RNA in a sample from a subject with cognitively spared Schizophrenia is indicative of Schizophrenia with cognitive deficit.

In another or further embodiment, an increased level of one or more small non-coding RNA(s) comprising the nucleotide sequence shown in any one of SEQ ID NOs: 3, 5, and 7 compared to the reference level(s) of the one or more small non-coding RNA(s) is indicative of Schizophrenia with cognitive deficit. For example, a log₂ fold increase of at least about 1 to at least about 1.3 compared to a reference level(s) of the one or more small non-coding RNA(s) in a sample from a subject with cognitively spared Schizophrenia is indicative of Schizophrenia with cognitive deficit.

In some embodiments, a log₂ fold increase of at least about 0.9, about 1, about 1.1 of the small non-coding RNA comprising the nucleotide sequence shown in SEQ ID NO: 1 compared to a reference level of the small non-coding RNA in a sample from a subject with cognitively spared Schizophrenia is indicative of Schizophrenia with cognitive deficit.

In some embodiments, a log₂ fold increase of at least about 1, about 1.1, about 1.2 of the small non-coding RNA comprising the nucleotide sequence shown in SEQ ID NO: 3 compared to a reference level of the small non-coding RNA in a sample from a subject with cognitively spared Schizophrenia is indicative of Schizophrenia with cognitive deficit.

In some embodiments, a log₂ fold increase of at least about 1, about 1.1, about 1.2, about 1.25, about 1.3 of the small non-coding RNA comprising the nucleotide sequence shown in SEQ ID NO: 5 compared to a reference level of the small non-coding RNA in a sample from a subject with cognitively spared Schizophrenia is indicative of Schizophrenia with cognitive deficit.

In some embodiments, a log₂ fold increase of at least about 0.9, about 0.95, about 0.96, about 0.97, about 0.98. about 0.99, about 1 of the small non-coding RNA comprising the nucleotide sequence shown in SEQ ID NO: 7 compared to a reference level of the small non-coding RNA in a sample from a subject with cognitively spared Schizophrenia is indicative of Schizophrenia with cognitive deficit.

In one or a further embodiment, a decreased level of a small non-coding RNA comprising the nucleotide sequence shown in SEQ ID NO: 8 compared to the reference level of the one or more small non-coding RNA is indicative of Schizophrenia with cognitive deficit. For example, a log₂ fold decrease of at least about 1.2, about 1.21, about 1.22, about 1.23, about 1.24, about 1.25 compared to a reference level of the small non-coding RNA in a sample from a subject cognitively spared Schizophrenia is indicative of Schizophrenia with cognitive deficit.

In one embodiment, the disease is cognitively spared Schizophrenia.

In one embodiment, a decreased level of at least one or more small non-coding RNA(s) comprising the nucleotide sequence shown in any one of SEQ ID NOs: 2, and 12 to 17 compared to the reference level(s) of the one or more small non-coding RNA(s) is indicative of cognitively spared Schizophrenia. For example, a log₂ fold decrease of at least about 0.4 to at least about 0.8 compared to a reference level(s) of the small non-coding RNA(s) in a sample from a normal subject is indicative of cognitively spared Schizophrenia.

In some embodiments, a log₂ fold decrease of at least about 0.4, about 0.41, about 0.42 of the small non-coding RNA comprising the nucleotide sequence shown in SEQ ID NO: 2 compared to a reference level of the small non-coding RNA in a sample from a normal subject is indicative of cognitively spared Schizophrenia.

In some embodiments, a log₂ fold decrease of at least about 0.6, about 0.61, about 0.62 of the small non-coding RNA comprising the nucleotide sequence shown in SEQ ID NO: 12 compared to a reference level of the small non-coding RNA in a sample from a normal subject is indicative of cognitively spared Schizophrenia.

In some embodiments, a log₂ fold decrease of at least about 0.7, about 0.71, about 0.72, about 0.73, about 0.74, about 0.75, about 0.76 of the small non-coding RNA comprising the nucleotide sequence shown in SEQ ID NO: 13 compared to a reference level of the small non-coding RNA in a sample from a normal subject is indicative of cognitively spared Schizophrenia.

In some embodiments, a log₂ fold decrease of at least about 0.6, about 0.61, about 0.62 of the small non-coding RNA comprising the nucleotide sequence shown in SEQ ID NO: 14 compared to a reference level of the small non-coding RNA in a sample from a normal subject is indicative of cognitively spared Schizophrenia.

In some embodiments, a log₂ fold decrease of at least about 0.5, about 0.51, about 0.52, about 0.53, about 0.54 of the small non-coding RNA comprising the nucleotide sequence shown in SEQ ID NO: 15 compared to a reference level of the small non-coding RNA in a sample from a normal subject is indicative of cognitively spared Schizophrenia.

In some embodiments, a log₂ fold decrease of at least about 0.4, about 0.41, about 0.42, about 0.43, about 0.44 of the small non-coding RNA comprising the nucleotide sequence shown in SEQ ID NO: 16 compared to a reference level(s) of the small non-coding RNA(s) in a sample from a normal subject is indicative of cognitively spared Schizophrenia.

In some embodiments, a log₂ fold decrease of at least about 0.4, about 0.41, about 0.42, about 0.43, about 0.44 of the small non-coding RNA comprising the nucleotide sequence shown in SEQ ID NO: 17 compared to a reference level of the small non-coding RNA in a sample from a normal subject is indicative of cognitively spared Schizophrenia.

In one embodiment, the disease is treatment resistant Schizophrenia.

In one embodiment, an increased level of at least one or more small non-coding RNA(s) comprising the nucleotide sequence shown in any one of SEQ ID NOs: 1, and 3 to 7 compared to the reference level(s) of the one or more small non-coding RNA(s) is indicative of treatment resistant Schizophrenia. For example, a log 2 fold increase of at least about 1.1 to at least about 2.6 compared to a reference level(s) of the small non-coding RNA(s) in a sample from a subject with Schizophrenia that is not treatment resistant is indicative of treatment resistant Schizophrenia.

In another embodiment, an increased level of a small non-coding RNA comprising the nucleotide sequence shown in SEQ ID NO: 1 compared to the reference level of the non-coding RNA is indicative of treatment resistant Schizophrenia. For example, a log₂ fold increase of at least about 2 compared to a reference level of the small non-coding RNA in a sample from a subject with Schizophrenia that is not treatment resistant is indicative of treatment resistant Schizophrenia.

In another or further embodiment, an increased level of one or more small non-coding RNA(s) comprising the nucleotide sequence shown in any one of SEQ ID NOs: 3, 5, and 7 compared to the reference level(s) of the one or more small non-coding RNA(s) is indicative of treatment resistant Schizophrenia. For example, a log 2 fold increase of at least about 2.3 to at least about 2.6 compared to a reference level(s) of the one or more small non-coding RNA(s) in a sample from a subject with Schizophrenia that is not treatment resistant is indicative of treatment resistant Schizophrenia.

In another or further embodiment, an increased level of at least one or more small non-coding RNA(s) comprising the nucleotide sequence shown in any one of SEQ ID NOs: 4 and 6 compared to the reference level(s) of the one or more small non-coding RNA(s) is indicative of treatment resistant Schizophrenia. For example, a log₂ fold increase of at least about 1.1 to at least about 1.3 compared to a reference level(s) of the small non-coding RNA(s) in a sample from a subject with Schizophrenia that is not treatment resistant is indicative of treatment resistant Schizophrenia.

In some embodiments, a log 2 fold increase of at least about 1.95, about 2, about 2.05 the small non-coding RNA comprising the nucleotide sequence shown in SEQ ID NO: 1 compared to a reference level of the small non-coding RNA in a sample from a subject with Schizophrenia that is not treatment resistant is indicative of treatment resistant Schizophrenia.

In some embodiments, a log₂ fold increase of at least about 2, about 2.1, about 2.2, about 2.3 of the small non-coding RNA comprising the nucleotide sequence shown in SEQ ID NO: 3 compared to a reference level of the small non-coding RNA in a sample from a subject with Schizophrenia that is not treatment resistant is indicative of treatment resistant Schizophrenia.

In some embodiments, a log₂ fold increase of at least about 1, about 1.1 about 1.2, about 1.3 of the small non-coding RNA comprising the nucleotide sequence shown in SEQ ID NO: 4 compared to a reference level of the small non-coding RNA in a sample from a subject with Schizophrenia that is not treatment resistant is indicative of treatment resistant Schizophrenia.

In some embodiments, a log₂ fold increase of at least about 2, about 2.5, about 2.6 of the small non-coding RNA comprising the nucleotide sequence shown in SEQ ID NO: 5 compared to a reference level of the small non-coding RNA in a sample from a subject with Schizophrenia that is not treatment resistant is indicative of treatment resistant Schizophrenia.

In some embodiments, a log₂ fold increase of at least about 1, about 1.1 of the small non-coding RNA comprising the nucleotide sequence shown in SEQ ID NO: 6 compared to a reference level of the small non-coding RNA in a sample from a subject with Schizophrenia that is not treatment resistant is indicative of treatment resistant Schizophrenia.

In some embodiments, a log 2 fold increase of at least about 2, about 2.1, about 2.2, about 2.3 of the small non-coding RNA comprising the nucleotide sequence shown in SEQ ID NO: 7 compared to a reference level of the small non-coding RNA in a sample from a subject with Schizophrenia that is not treatment resistant is indicative of treatment resistant Schizophrenia.

In one embodiment, a decreased level of a small non-coding RNA(s) comprising the nucleotide sequence shown in any one of SEQ ID NOs: 18 to 20 compared to the reference level(s) of the small non-coding RNA(s) is indicative of treatment resistant Schizophrenia.

In some embodiments, a log 2 fold decrease of at least about 0.8, about 0.9, about 1, about 1.1, about 1.2, about 1.25, about 1.26, about 1.27, about 1.28, about 1.29, about 1.3, about 1.35, about, 1.36, about 1.37, about 1.38, about 1.39, about 1.4 of a small non-coding RNA comprising the nucleotide sequence shown in SEQ ID NO: 18 compared to a reference level(s) of the small non-coding RNA(s) in a sample from a subject with Schizophrenia that is not treatment resistant, or from a pooled sample from a subject with Schizophrenia that is not treatment resistant and a normal subject is indicative of treatment resistant Schizophrenia.

In some embodiments, a log₂ fold decrease of at least about 1, about 1.1 about 1.2, about 1.3 of a small non-coding RNA comprising the nucleotide sequence shown in SEQ ID NO: 19 compared to a reference level of the small non-coding RNA in a pooled sample from a subject with Schizophrenia that is not treatment resistant and a normal subject is indicative of treatment resistant Schizophrenia.

In some embodiments, a log₂ fold decrease of at least about 0.7, about 0.75, about 0.8, about 0.81, about 0.82, about 0.83, about 0.84, about 0.85 of a small non-coding RNA comprising the nucleotide sequence shown in SEQ ID NO: 20 compared to a reference level of the small non-coding RNA in a pooled sample from a subject with Schizophrenia that is not treatment resistant and a normal subject is indicative of treatment resistant Schizophrenia.

In other embodiments, the reference level is the level of the small non-coding RNA(s) in a pooled sample from a subject with Schizophrenia that is not treatment resistant and a normal subject.

In one embodiment, the disease is Schizophrenia with depression.

In one embodiment, an increased level of a small non-coding RNA comprising the nucleotide sequence shown in SEQ ID NO: 4 compared to the reference level(s) of the small non-coding RNA is indicative of Schizophrenia with depression.

In one embodiment, a log₂ fold increase of at least about 0.8, about 0.9, about 1.0, about 1.1 compared to a reference level of the small non-coding RNA in a sample from a non-depressed subject with Schizophrenia or from a normal subject, or from a pooled sample from a non-depressed subject with Schizophrenia and a normal subject is indicative of Schizophrenia with depression.

In some embodiments, the method further comprises measuring total circulating neuronal origin extracellular vesicle small non-coding RNA level.

In one embodiment, a log 10 fold decrease of at least about 0.3 total circulating neuronal origin extracellular vesicle small non-coding RNA compared to the reference level of total circulating neuronal origin extracellular vesicle small non-coding RNA in a sample from a normal subject is indicative of Schizophrenia or a predisposition to Schizophrenia.

In one embodiment, a log 10 fold decrease of at least about 0.3 total circulating neuronal origin extracellular vesicle small non-coding RNA compared to a reference level of total circulating neuronal origin extracellular vesicle small non-coding RNA in a sample from a normal subject is indicative of Schizophrenia with cognitive deficit.

In another embodiment, a log 10 fold decrease of at least about 0.3 total circulating neuronal origin extracellular vesicle small non-coding RNA compared to a reference level of total circulating neuronal origin extracellular vesicle small non-coding RNA in a sample from a normal subject is indicative of Schizophrenia with spared cognition.

In some embodiments, the method further comprises a psychological, behavioural, physiological and/or genetic assessment of the subject.

In some embodiments, the psychological assessment determines the presence and/or level of cognitive impairment.

In some embodiments, the method further comprises selecting a treatment or modifying a treatment for the disease state or the predisposition to the disease state, based on the diagnosis of disease state.

In some embodiments, the treatment selected or modified may target signaling pathways important to synaptic plasticity, for example, Brain-derived neurotrophic factor (BDNF) signaling, or cAMP signaling, or signaling involving NOVA regulated synaptic proteins.

In further embodiments, the treatment selected or modified may target signaling pathways, for example, Erythropoietin (EPO) signaling, or Morphine addiction, or Cholinergic synapse, or Phosphodiesterases in neuronal function and GABAergic synapse.

In some embodiments, the method further comprises administering to the subject a therapeutically effective amount of an anti-disease state therapeutic.

The present disclosure also provides a method for preparing an extracellular vesicle fraction enriched for neuron derived extracellular vesicles from a sample for analysing small non-coding RNA involved in a disease state, comprising:
(a) providing a biological sample;
(b) contacting the sample with a microtubule associated protein (MAP) binding agent to selectively bind extracellular vesicles expressing MAP; and
(c) isolating the bound extracellular vesicles.

In one embodiment, the method further comprises:
(d) extracting RNA from the extracellular vesicles; and
(e) analysing the level of the one or more small non-coding RNA to a reference level of the one or more small non-coding RNA, wherein an increased or decreased level of the one or more small non-coding RNA compared to the reference level is indicative of a disease state or a stage of development of a disease state.

In one embodiment, the MAP is MAP1B.

In one embodiment, the MAP binding agent is immobilised to a non-porous support.

In one or a further embodiment, isolating the bound extracellular vesicles comprises:
(i) removing the unbound fraction
(ii) releasing the extracellular vesicles from the binding agent.

In one or a further embodiment, the MAP binding agent is an antibody or binding fragment thereof.

The present disclosure also provides a method for the isolation of neuron derived extracellular vesicles from a blood sample, comprising:
(a) contacting the sample with a microtubule associated protein (MAP) binding agent to selectively bind extracellular vesicles expressing MAP1B; and
(b) isolating the bound extracellular vesicles.

The present disclosure also provides a population of neuron derived extracellular vesicles, wherein the extracellular vesicles comprise one or more small non-coding RNA comprising a sequence selected from any one of SEQ ID NOs: 2, 6, 9, 21, or 29 to 51.

The present disclosure also provides a population of neuron derived extracellular vesicles, wherein the extracellular vesicles comprise one or more small non-coding RNA comprising a sequence selected from any one of SEQ ID NOs: 30-32.

In a further embodiment, the extracellular vesicles comprise one or more additional small non-coding RNA comprising a sequence selected from any one of SEQ ID NOs: 2, 6, 9, 21, 29, or 33 to 51.

In some embodiments, the relative abundance of the one or more small non-coding RNA is about 0.013 to about 0.027.

The present disclosure also provides a population of neuron derived extracellular vesicles, wherein the extracellular vesicles comprise one or more seed sequences comprising a sequence selected from any one of SEQ ID NOs: 63-65. These sequences may target synaptic genes associated with intellectual disability and may be used to modulate treatment thereof.

In some embodiments, the extracellular vesicles are isolated according to a method of the disclosure.

The present disclosure also provides a kit for the isolation of neuron derived extracellular vesicles from a blood sample comprising:
(a) a microtubule associated protein (MAP) binding agent; and
optionally, one or more agents selected from a solid support, wash buffer, elution buffer.

In some embodiments the population of neuron derived extracellular vesicles may be used for screening putative drugs.

The present disclosure provides a method for a drug screening assay comprising the steps of:
(a) providing a population of extracellular vesicles of the disclosure;
(b) contacting the extracellular vesicles with a drug;
(c) monitoring the effect of the drug on modulation of the one or more sequences (e.g., small non-coding RNA or seed sequence).

In some embodiments, the assay can be used to screen for drugs that modulate the blood brain barrier.

The present disclosure is not to be limited in scope by the specific examples described herein, which are intended for the purpose of exemplification only. Functionally equivalent products, compositions and methods are clearly within the scope of the present disclosure.

Any example/embodiment of the present disclosure herein shall be taken to apply mutatis mutandis to any other example/embodiment of the disclosure unless specifically stated otherwise.

### BRIEF DESCRIPTION OF DRAWINGS

FIG. 1 Neuronal origin miRNA, from serum extracellular vesicles, consistently observed across samples. a) Heat map for the expression of each miRNA (n=105) in each sample (n=477) is represented by a square. Yellow=high expression, blue=low expression. Sample labels in the heat map are omitted due to the large number of samples. Labels for miRNA in the heat map are limited to every fifth label. miRNA expression is normalised counts per million, converted to z-score. Heat map constructed by ordering the distance matrix to minimise the Hamilton path length. b) Principal components analysis demonstrates hsa-miR-19b-3p and hsa-miR-19a-3p together (top panel) and individually (middle and bottom panels) explain the most variation in dimension one, represented as the highest quality variables on the factor map by cos 2 (squared coordinates). c) Given that hsa-miR-19b-3p and hsa-miR-19a-3p are transcribed from the same primary transcript and show evidence of correlated expression (Table 5), PCA was repeated without hsa-miR-19a-3p (b, middle panel) and then without hsa-miR-19b-3p (b, bottom panel), in each case showing a small reduction in proportion of variance retained in dimension one.

FIG. 2 miRNA dysregulated in immuno-fractionated serum extracellular vesicles from schizophrenia subjects. Total RNA was recovered from anti-MAP1B enriched serum extracellular vesicles from schizophrenia subjects (n=221) and non-psychiatric controls (n=256). Small RNA libraries were constructed for each subject and sequenced on NovaSeq instrument for 100 cycles of paired-end reads. Differential expression analysis was conducted using edgeR with adjustment for batch, gender and age. a) Significant differential expression (FDR<0.1) of neuronal miRNA was observed for schizophrenia subjects compared to non-psychiatric controls (whole cohort), b) cognitive deficit subtype of schizophrenia (CD) compared to non-psychiatric controls and c) cognitive deficit subtype of schizophrenia compared to cognitively spared subtype of schizophrenia (CS).

FIG. 3 Association of gene sets from dysregulated miRNA in schizophrenia subjects. a) Forest plot of MAGMA gene-set association results for the three sets of predicted targets for the differentially expressed miRNA between each group. SZ=schizophrenia, CD=cognitive deficit, CS=cognitively spared. Each panel displays the results constructing a model using liberal or conservative definitions the boundaries for each gene, as well as a model with conservative boundaries additionally covaried for cortical gene expression for each gene. Conservative boundaries extend the gene 5 kb upstream and 1.5 kb downstream to capture regulatory variation, whilst liberal boundaries are 35 kb and 10 kb upstream and downstream, respectively. The MAGMA beta-coefficients for the gene-set term are plotted with the error bars representing the standard error of the coefficient. b) QQ-plots of residualised genic Z values from a null model without the gene-set term for the inhibitory and excitatory neuron genesets (conservative boundaries). Black points denote the 25th, 50th, and 75th quantiles, whilst the dotted line is the one-sided, upper 95% confidence band of deviation from the diagonal by chance alone. Points are shaded red to indicate if they fall above the confidence band or grey otherwise.

FIG. 4 Cognitive deficit associated miRNA are enriched with target genes associated with neural processes. Predicted targets of miRNA significantly dysregulated in schizophrenia subjects with cognitive deficit compared to non-psychiatric controls eleven miRNA were generated using TargetScanHuman and filtered to retain transcripts with binding sites for at least three of the eleven miRNA. The gene list was mapped to predefined pathways and tested (hypergeometric) for overrepresentation using Consensus Path Database (CPDB). Among the significantly enriched pathways are “Cholinergic synapse” (q-value=0.001), “Nervous system development” (q-value=0.002), “Neurotrophin signaling pathway” (q-value=0.003), “Erythropoietin activates Phosphoinositide-3-kinase” (q-value=0.009) and “Reelin signaling pathway (q-value=0.010).

FIG. 5 Differential expression of neuronal origin miRNA in serum extracellular vesicles from subjects with treatment resistant schizophrenia (TRS). Differential expression was determined using edgeR with adjustment for batch, gender and age. Significant differential expression (FDR<0.1) of neuronal miRNA was observed for TRS (n=42) compared to non-TRS (n=179). a) To enable comparison with the substantive analysis (miRNA differentially expressed in schizophrenia and cognitive subtypes compared to non-psychiatric controls) the same set of miRNA (105 miRNA) were analysed. b) A larger set of miRNA (196 miRNA) were analysed. Significant differential expression (FDR<0.1) of neuronal miRNA was also observed, with the inclusion of non-psychiatric controls, for TRS (n=42) compared to non-TRS (n=435) c) adjusted for case control status and d) unadjusted for case control status.

FIG. 6 Enrichment for neuronal-origin EV's from serum. Schematic summary of immunofractionation of serum to enrich for neuronal origin EV's and subsequent extraction of neuronal RNA for small RNA sequencing. a) Neurons, and other brain cell types, release EV's to the extracellular milieu both constitutively and in response to activity (refer to introduction). Released EV's, present in the interstitial fluid that surrounds neural cells, exit the brain parenchyma to reach the blood through two possible routes i) the vascular pathway, guarded by the blood-brain-barrier (BBB) and II) the cerebrospinal fluid (CSF) pathway via perivascular and perineural spaces. Given the relatively large size and negative charge of EV's, their transport to the vasculature via the BBB is uncertain, although there are reports of blood to brain transit of EV's via vesicle transcytosis (Banks et al, 2020). The CSF pathway is the more likely route of exit, although the specific mechanisms and relative contributions are unclear (for a recent review, see Kaur et al., 2021). Nevertheless, EV's have been recovered from adult human postmortem brain tissue, CSF and peripheral biofluids (refer to discussion) and proteomics of neuron-derived plasma EV's have informed Alzheimer's disease related pathology (Fiandaca et al., 2015; Goetzl et al., 2015; Kapogiannis et al., 2015). b) Whole blood was obtained from study participants during clinical interview, processed to serum and stored at −80° C. c) As described in methods, serum aliquots (100 μL) were incubated with anti-MAP1B coupled magnetic beads allowing removal of the supernatant (containing unbound EV's, including those of non-neuronal origin) while leaving an enriched fraction of relatively homogeneous neuronal origin EV's for RNA extraction and small RNA sequencing.

FIG. 7 Serum EV's enriched for neuronal origin demonstrate a distinct miRNA profile. Venn diagram of human mature miRNA identified in total serum EV's (‘Total’), neuronal origin enriched serum EV's (‘Neuronal enriched’) and serum EV's depleted of neuronal origin EV's (‘Neuronal depleted’). The detection of three miRNA (hsa-miR-615-3p, hsa-miR-424-3p and hsa-miR-155-5p) is specific to the neuronal enriched fraction.

FIG. 8 Distinct miRNA profile from neuronal enriched compared to neuronal depleted serum EV's. Relative abundance of each miRNA was calculated as miRNA count/total miRNA counts. The miRNA from neuronal enriched and neuronal depleted fractions were combined and ranked by relative abundance and the top one-third abundant miRNA are shown, corresponding to relative abundance of >0.0127.

FIG. 9 Differential expression of neuronal origin miRNA in serum extracellular vesicles from subjects with depression. Differential expression was determined using edgeR with adjustment for batch, gender and age. Neuronal miRNA differential expression was observed for schizophrenia subjects with depression (n=60) compared to non-depressed schizophrenia subjects and non-psychiatric control subjects (n=417). Increased expression of miR-3178 was significant (FDR<0.05).

FIG. 10 Schizophrenia associated miRNA from neuronal origin serum extracellular vesicles are highly expressed in the human brain. miRmine data was downloaded for ten human brain datasets. miRmine mature miRNA were ranked by average Reads per Million (RPM), where rank 1 corresponds to highest expression in brain. The five most highly expressed miRNA in human brain include miR-92a-3p and miR-486-5p, both of which are significantly reduced in cognitive deficit subtype schizophrenia while miR-486-5p is significantly reduced in the combined schizophrenia group.

FIG. 11 Reduced circulating neuronal origin miRNA in schizophrenia subjects. Library sizes were normalised by the trimmed mean of M values (TMM), as described in (2) and plotted as log 10 of total miRNA counts. (A) Comparison of the distribution of effective library sizes for non-psychiatric comparison subjects (CO, n=256) and schizophrenia subjects (SZ, n=221) demonstrates reduced circulating miRNA in schizophrenia (Wilcoxon rank sum test, P=4.385e-08). (B) Pairwise comparison demonstrates both schizophrenia subjects with severe cognitive deficits (szCD, n=111) and schizophrenia subjects with spared cognition (szCS, n=110) have decreased miRNA with respect to comparison subjects (szCD: P=2.5e-05; Wilcoxon rank sum test, szCS: P=1.8e-05; Wilcoxon rank sum test). P values adjusted for multiple testing by false discovery rate method.

FIG. 12 Synaptic plasticity pathways with druggable protein targets. (A) Selected pathways with enrichment for genes targeted by miRNA differentially expressed in schizophrenia subjects with severe cognitive deficits (szCD). (B) Approved medications that target genes from the five selected pathways enriched for protein drug targets (adjusted P value<0.05).

FIG. 13 Phosphodiesterase interactions form the largest group of druggable targets associated with cognitive deficit schizophrenia miRNA. Gene lists from selected pathways (FIG. 12A) were filtered for minimum interaction score of 0.7000 (high confidence) and clustered (Markov cluster algorithm (MCL) using inflation parameter 4) as implemented in STRING. The largest cluster is composed of genes from the phosphodiesterases in neuronal function pathway (red nodes), the second largest cluster are predominantly genes from EPO signaling pathway (peach nodes) and the third largest cluster is equally composed of genes from EPO signaling and cholinergic synapse (brown nodes).

FIG. 14 Consistently dysregulated miRNA. (A) miR-1246 was the only miRNA consistently dysregulated across all contrasts (CD v CS, CD v CO, TRS v nonTRS and SZ v CO). miR-4521, miR-5100 and miR-7704 were the next most consistently dysregulated (CD v CS, CD v CO and TRS v nonTRS), followed by miR-203a-3p and miR-3178 (CD v CO and TRS v nonTRS), and finally miR-451a (CD v CS and CD v CO) and miR-486-5p (CD v CO and SZ v CO). (B) Differential expression of miR-1246 (log 2 fold-change) across all comparisons. Schizophrenia with severe cognitive deficits (CD, n=111), Schizophrenia with spared cognition (CS, n=110), non-psychiatric comparison subjects (CO, n=256), treatment resistant schizophrenia (TRS, n=42) and subjects who are not treatment resistant include cases and comparison (nonTRS, n=435).

FIG. 15 miR-1246 target brain enriched genes. Predicted targets of hsa-miR-1246 were determined with TargetScan and filtered to include high confidence target genes based on binding site efficacy as cumulative weighted context score less than-0.2. (A) Target genes are specifically enriched in the brain and (B) significantly overlap with miR-137 target genes.

FIG. 16 Validation of anti-MAP1B with human serum. Relative RNA expression increased >15-fold when anti-MAP1B coupled beads (“anti-MAP1B (+)”) were used to enrich for neuronal origin serum EVs compared to nil antibody control (“anti-MAP1B (−)”).

### DETAILED DESCRIPTION

Before describing the present invention in detail, it is to be understood that the terminology used herein is for the purpose of describing particular embodiments only and is not intended to be limiting.

As used in this specification and the appended claims, the singular forms “a”, “an” and “the” include plural referents unless the content clearly dictates otherwise.

Throughout the description and claims of the specification, the word “comprise” and variations of the word, such as “comprising” and “comprises”, is not intended to exclude other additives, components, integers or steps.

Unless specifically defined otherwise, all technical and scientific terms used herein shall be taken to have the same meaning as commonly understood by one of ordinary skill in the art

All publications cited herein are entirely incorporated herein by reference. Publications refer to any scientific or patent publications, or any other information available in any media format, including all recorded, electronic or printed formats. The following references are entirely incorporated herein by reference: Ausubel, et al., ed., Current Protocols in Molecular Biology, John Wiley & Sons, Inc., NY, N.Y. (1987 including all updates until present); Sambrook, et al., Molecular Cloning: A Laboratory Manual, 2nd Edition, Cold Spring Harbor, N.Y. (1989); Harlow and Lane, Antibodies, a Laboratory Manual, Cold Spring Harbor, N.Y. (1989); Colligan, et al., eds., Current Protocols in Immunology, John Wiley & Sons, Inc., NY (1994 including all updates until present); Colligan et al., Current Protocols in Protein Science, John Wiley & Sons, NY, N.Y., (1997 including all updates until present).

A reference herein to a publication which is given as prior art is not to be taken as admission that publication was known or that the information it contains was part of the common general knowledge as at the priority date of any of the claims.

“Evaluation” in the context of the present disclosure relates to the assessment of the severity of the disease or condition in a subject.

“Diagnosis” in the context of the present disclosure relates to the recognition and detection (e.g., early detection) of a disease or condition in a subject and may also comprise differential diagnosis and pre-disposition of the subject to develop the disease or clinical condition. A diagnosis may include an assessment of the degree of disease/condition severity (e.g., “low” to “high”), current state of disease progression (e.g., “early”, “middle,” or “late” stages), or include a comparative assessment to an earlier diagnosis (e.g., “advancing”, “stable”, or in “remission”).

“Prognosis” denotes a prediction of how a subject's (e.g., a patient's) disease or condition will progress. This may include a prediction of the chance of recovery or the chance of an adverse outcome for said subject. Prognosis may also include a prediction of disease/condition response to treatment.

The term “score” in the context of the present disclosure refers to a rating, expressed numerically, based on the specific achievement or the degree to which certain qualities or conditions are manifest.

The term “treatment stratification” in the context of the present disclosure refers to the choice and/or adjustment of a therapeutic treatment of the subject.

The term “risk stratification” in the context of the present disclosure refers to a probability of specified outcomes for a subject.

As used herein the term “subject” refers to animals, preferably mammals, and, more preferably, humans. In some embodiments, the subject is a dog, a cat, a horse, a cow, other farm animals, or a rodent (e.g., mice, rats, guinea pig, etc.). The term “subject,” “patient,” and “individual” are used interchangeably herein.

The term “patient” as used herein includes a living human or non-human subject that is receiving medical care or that should receive medical care due to a disease or condition. This includes subjects with no defined illness or observable symptoms of a disease or condition who are being investigated for signs of pathology. Thus, the methods described herein are applicable to both, human and veterinary disease.

In some examples, the term “subject” refers to subjects at risk of or with symptoms of neurodegenerative disease and/or neuropsychiatric diseases and mood disorders (including schizophrenia, anxiety, bipolar disorder; manic depression and the like (e.g., problems with movement or mental functioning, etc.).

As used herein “mood disorders” are conditions characterized by a disturbance in the regulation of mood, behavior, and affect. “Mood disorders” can include depression, anxiety, schizophrenia, bipolar disorder, manic depression and the like.

As used herein “depression” includes depressive disorders or depression in association with medical illness or substance abuse in addition to depression as a result of sociological situations. Patients defined as having depression may be diagnosed mainly on the basis of clinical symptoms including a depressed mood episode wherein a person displays a depressed mood on a daily basis for a period of greater than, for example, 2 weeks. A depressed mood episode may be characterized by sadness, indifference, apathy, or irritability and is usually associated with changes in a number of neurovegetative functions, including sleep patterns, appetite and weight, fatigue, impairment in concentration and decision making. Patients taking an antidepressant are deemed to have depression or depressive disorder.

As used herein, Schizophrenia refers to a psychiatric disorder that typically manifests in the years between late adolescence and early adulthood. Currently, diagnosis is made in the clinical setting based on operational criteria such as the nature and duration of symptoms, medical history and family history. Symptoms are broadly classified as; positive including hallucinations and delusions; negative for example, apathy and social withdrawal; and cognitive impairments. Cognitive and negative symptoms are not ameliorated to the same extent as positive symptoms with current pharmacotherapies and contribute to greater functional impairment. Although it starts out as a neurodevelopmental disorder, Schizophrenia becomes neurodegenerative after onset, with each new psychotic episode leading to further damage. As such, the term “neurodegenerative disease” as used herein, is intended to also include Schizophrenia.

As used herein, “neurodegeneration is a slow and progressive loss of neuronal cells in specified regions of the brain and is the main pathologic feature of Alzheimer's disease, Parkinson's disease, Huntington's disease (HD), epilepsy and other various neurodegeneration-initiated cerebral failures. The underlying pathophysiology of these neurodegenerative diseases is oxidative stress from impaired mitochondrial function, deposition of aggregated proteins, neuroinflammation, and activation of apoptotic factors.

As used herein, “neurodegenerative disease” refers to characterized complex and serious medical conditions, which principally affect the neurons in the human brain. Such conditions lead to the disorders of the central nervous system (CNS), which eventually results in the progressive loss of neural tissues including death of neurons.

As a mental illness, schizophrenia is principally a disorder of the central nervous system (CNS), particularly neuronal cell types and gene expression from postmortem human brain tissue provides evidence of transcriptional dysregulation in schizophrenia. Yet, among the many differentially expressed genes there is often a lack of replication of findings and more importantly, the miss expressed genes do not yield a coherent explanation for the observed phenotypes. This suggests that additional factors between genotype and phenotype are involved.

A long-standing concern in the field is that clinical presentation is extremely variable and the underlying biology driving this heterogeneity is not adequately captured by current diagnostic instruments, such as the Diagnostic and Statistical Manual of the American Psychiatric Association (DSM) and International Classification of Diseases (ICD). This has led to a need for discerning molecular signatures of psychiatric illnesses such that diagnosis and treatment are biologically informed.

In some examples, the subject is identified as being at risk of developing a disease or condition, for example, a neurodegenerative disease or neuropsychiatric disease that could result from disease, trauma, or a medical procedure. In some examples, the subject is identified as being at risk of developing a neurodegenerative or neuropsychiatric disease that could result from endocrine conditions, oxidative stress, infection and inflammation, nutrition, vascular conditions, depression, psychological trauma, head trauma, and tumors. In some examples, illicit/psychoactive drugs may be associated with increased risk of developing a neurodegenerative disease or neuropsychiatric disease.

In some examples, the methods include the step of identifying a subject with at risk for developing or having a sign or symptom of a disease or condition, for example, a neurodegenerative disease or neuropsychiatric disease.

As used herein, “disease”, “disorder”, “condition” and the like, are used interchangeably and have meanings ascribed to each and all of such terms.

The term “apoptotic bodies” refers to vesicles produced by dying cells having a diameter between 50-5000 nm. Apoptotic bodies contain exposed phosphatidylserine on their membranes, and their major protein markers include histones, TSP, and C3b. A notable distinction between apoptotic bodies and exosomes and microvesicles is that apoptotic bodies also contain fragmented DNA and cell organelles from their host cell.

The term “exosome” refers to cell-derived vesicles having a diameter of between about 50 and 160 nm, preferably a diameter of about 100-160 nm, for example, a diameter of about 130 nm, 135 nm, 140 nm, 145, nm, 150 nm or 155 nm, as determined by NTA, nanoparticle tracking analysis.

Exosomes include specific surface markers, including surface markers such as tetraspanins, for example, CD9, CD37, CD44, CD53, CD63, CD81, CD82 and CD151; targeting or adhesion markers such as syntenin, integrins, ICAM-1, EpCAM and CD31; membrane fusion markers such as annexins, TSG101, ALIX; and other exosome transmembrane proteins such as Rab5b, HLA-G, HSP70, LA1VIP2 (lysosome-associated membrane protein) and LIMP (lysosomal integral membrane protein). Normally used exosome markers include syntenin, Alix, Tsg101, tetraspanins (CD81, CD63, CD9), and flotillin, preferably Tsg101 and syntenin.

The term “microvesicles” refers to cell-derived vesicles having a diameter of between about 50 and 1000 nm, preferably a diameter of about 50-500 nm, for example, a diameter of about 100 nm, 150 nm, 200 nm, 250 nm, 300 nm, 350 nm, 400 nm, 450 nm, or 500 nm. Microvesicles include specific surface markers not present in other vesicles, including surface markers such as integrins, selectins, CD40.

The term “exosomes” refers to vesicles produced from intraluminal vesicles that form within multivesicular bodies (MVBs, or multivesicular endosomes), fusion of the MVB at the plasma membrane releases these vesicles into the extracellular milieu where they are known as exosomes.

An “effective amount” refers to at least an amount effective, at dosages and for periods of time necessary, to achieve the desired result. For example, the desired result may be a therapeutic or prophylactic result. An effective amount can be provided in one or more administrations. In some examples of the present disclosure, the term “effective amount” is meant an amount necessary to effect treatment of disease pathology in an animal as described herein. The effective amount may vary according to the weight, age, sex, health and/or physical condition and other factors relevant to the animal being treated. Typically, the effective amount will fall within a relatively broad range (e.g., a “dosage” range) that can be determined through routine trial and experimentation. Accordingly, this term is not to be construed to limit the disclosure to a specific quantity. The effective amount can be administered in a single dose or in a dose repeated once or several times over a treatment period.

### Extracellular Vesicles

Extracellular vesicles useful in the methods of the disclosure include exosomes and microvesicles. Exosomes are nanometer-sized vesicles of endocytic origin that form by inward budding of the limiting membrane of multivesicular endosomes. Their size is typically equivalent to that of the intraluminal vesicle within multivesicular endosomes (50-160 mn). Due to their endocytic origin exosomes are commonly enriched in endosome-associated proteins such as Rab GTPases, SNAREs, Annexins, and flotillin. Some of these proteins (e.g. Alix and Tsg101) are normally used as exosome markers. Tetraspanins are a family of membrane proteins known to cluster into microdomains at the plasma membrane. These proteins are abundant in exosomes and considered to be markers as well. Microvesicles bud from the cell surface and their size may vary between 50-1,000 nm. Common protein markers used to define these vesicles are selectins, integrins and the CD40 ligand.

Extracellular vesicles are produced by many different types of cells including immune cells such as B lymphocytes, T lymphocytes, dendritic cells (DCs). Extracellular vesicles are also produced, for example, by glioma cells, platelets, reticulocytes, neurons, glia, intestinal epithelial cells and tumour cells. Extracellular vesicles for use in the methods of the disclosure can be derived from any suitable cell, preferably neurons. Neurons, and other brain cell types, typically release extracellular vesicles to the extracellular milieu both constitutively and in response to activity. Released extracellular vesicles, present in the interstitial fluid that surrounds neural cells, can exit the brain parenchyma to reach the blood.

Extracellular vesicles can therefore be isolated from tissues or physiological fluids, such as plasma, urine, amniotic fluid and malignant effusions. In some embodiments, the extracellular vesicles are isolated from brain tissues or gut tissues. In alternate embodiments, the extracellular vesicles are isolated from peripheral biofluids, preferably circulating serum.

In other embodiments, extracellular vesicles are isolated from whole blood, serum, or plasma of a patient with a neurodegenerative disease or neuropsychiatric disease.

### Isolation of Extracellular Vesicles

Multiple different methods are known in the art to isolate extracellular vesicles from different samples such as conditioned cell culture medium, serum, blood, and urine. Once isolated, extracellular vesicles can be characterized by technology such as nanoparticle tracking analysis, electron microscopy, density gradients, dynamic light scattering, and nanoscale flow cytometry.

In some embodiments, the extracellular vesicles are isolated by centrifugation. For example, particles with a high buoyant density are first sedimented, such as cells, cell debris, apoptotic bodies, and aggregates of biopolymers. In order to reduce losses caused by co-sedimentation and to decrease contamination of the preparations with the products of cell lysis, this step typically includes several substeps, for example, centrifugation at 300-400×g for about 10 min to sediment a main portion of the cells, at 2000×g to remove cell debris, and at 10,000×g to remove the aggregates of biopolymers, apoptotic bodies, and the other structures with the buoyant density higher than that of extracellular vesicles. Extracellular vesicles contained in the resulting supernatant can be sedimented by ultracentrifugation at, for example, >100,000×g (100,000-200,000×g) for about 2 hours. The non-extracellular vesicle proteins in the extracellular vesicle pellet can be removed by suspending followed by repeated ultracentrifugation. The obtained extracellular vesicle preparation can be further purified and the isolated microparticles selected according to their size by microfiltration of suspension using filters with pore diameters of, for example, 0.1, 0.22, or 0.45 μm.

In alternate embodiments, low-speed centrifugation (<10,000×g) can be used to remove cells and cell debris or centrifugation at 16,000×g. Different spinning speeds (100,000 to 200,000×g) can also be used for final extracellular vesicle sedimentation.

Ultracentrifugation can be used isolate the extracellular vesicle fraction with a size of 20-250 nm. The isolated extracellular vesicles display one or more of the following markers: CD9, CD63, CD81, TSG101, syntenin, Alix, Flotillin-1, AQP2, and FLT1.

In some embodiments, density gradient ultracentrifugation is used in order to increase the efficiency of particle separation according to their buoyant density. This method enables separation of subcellular components, such as mitochondria, peroxisomes, and endosomes and is typically used to isolate microvesicles. Density gradient ultracentrifugation utilizes two methods for formation of the gradient, namely, a continuous density gradient (formed either during centrifugation or upfront) or a stepwise gradient (the density increases in a discrete manner), a sucrose cushion. A long high-speed centrifugation results in concentration of the exosome-like vesicles in a band with close densities (exosomes, approximately 1.1-1.19 g/ml, but varying depending on the extracellular vesicle content); thus, extracellular vesicles can be separated from proteins and nucleoproteins. The extracellular vesicles isolated by ultracentrifugation typically express different exosomal markers, such as CD9, CD63, CD81, TSG101, syntenin, Alix, Flotillin-1, AQP2, HSP70, and FLT1 as well as some amount of non-extracellular vesicle proteins.

Differential ultracentrifugation can be used to isolate the extracellular vesicle fraction with a size of 50-160 nm. In some embodiments, the isolated extracellular vesicle fraction does not comprise any vesicles over 200 nm.

There are numerous protocols known in the art for extracellular vesicle isolation that utilize the separation of micro/nanoparticles according to their size, including ultrafiltration, hydrostatic dialysis, and gel filtration.

Commercial membrane filters have pores of various diameters with a narrow range of pore size distribution, which simplifies isolation of the particles with a specified size. In some embodiments, a method used for extracellular isolation can be supplemented with micro- or ultrafiltration. Ultrafiltration may alternate successive ultracentrifugation stages or it can be an additional step in gel filtration chromatography.

When isolating extracellular vesicles by microfiltration, the filters with pore diameters of 0.8, 0.45, 0.22, and 0.1 μm are typically used; such filters retain the particles with diameters of over 800, 450, 220, and 100 nm, respectively (+/−20%). Larger particles are removed first (by filters with pore diameters of, for example, 0.8 and 0.45 μm) and the particles with a size smaller than the target extracellular vesicles are separated from the filtrate at the next stage (by filters with pore diameters of, for example, 0.22 and 0.1 μm). Thus, the extracellular vesicle fraction of a specified size is concentrated.

Protocols utilizing ultrafiltration in combination with centrifugation and ultracentrifugation can be used to separate individual fractions of large microvesicles and exosomes in a selective manner. Microfiltration through the filters with a pore diameter of, for example, 0.65 μm and centrifugation at, for example, 10,000×g give microvesicles, while successive filtration using, for example, 0.65, 0.45, 0.22, and 0.1 μm filters and ultracentrifugation allows for selective isolation of exosomes.

The difference in the composition of isolated fractions can be confirmed by cryoelectron microscopy, particle size analysis, flow cytometry, and/or western blot assays for Alix, TSG101, CD63, CD81, and EpCAM proteins.

Another method for selective isolation of exosomes is the successive ultrafiltration comprising several stages, namely, filtration using, for example, 0.1 μm filter (e.g., Millipore Express (PES) membrane Stericup Filter Unit with a low affinity for proteins) and five-time tangential flow filtration using, for example, 0.1 μm filter (e.g., 100 nm TrackEtch filter, Millipore, United States). The first stage separates the exosomes and microvesicles from the very large particles; tangential flow filtration cleans the specimen from small-sized contaminants (mainly proteins), and the final step selectively separates exosomes and microvesicles.

In other embodiments, the extracellular vesicles can be isolated by gel filtration (size exclusion chromatography). Gel filtration makes it possible to separate the molecules differing in their hydrodynamic radius and is widely used for separation of biopolymers (proteins, polysaccharides, proteoglycans, etc.). Pretreatment and concentration of extracellular vesicle samples by ultracentrifugation or ultrafiltration are typically required in order to obtain the extracellular vesicle preparations free of proteins and lipoprotein impurities.

In some embodiments, extracellular vesicles are isolated by utilising methods that change extracellular vesicle solubility and/or aggregation. Extracellular vesicles can be precipitated using PEG solutions. This method utilizes a decrease in the solubility of compounds in the solutions of superhydrophilic polymers, PEGs. The procedure comprises mixing of the sample and polymer solution, incubation, and sedimentation of extracellular vesicles by low-speed centrifugation (for example, at 1500×g). The extracellular vesicles can be resuspending in, for example, PBS. The size of the extracellular vesicles isolated with PEG is comparable to the particles isolated by ultracentrifugation, ultrafiltration, and gel chromatography.

In some embodiments, a positively charged molecule, for example, protamine, can be used to aggregate and isolate extracellular vesicles. The protamine can be used in combination with PEG, for example PEG 35,000 Da. For example, the sample is first centrifuged (1500-3000×g). Then biological samples are mixed with precipitating solutions (4:1), such as 1-0.1 mg/ml protamine, 0.2 g/ml PEG 35,000, or a mixture of protamine and PEG. The resulting solution is incubated overnight and centrifuged at, for example, 1500×g (30 min, 22° C.). The pellet is then suspended in buffer and gel-filtered on, for example, a Sephadex G-100 (e.g., GE Healthcare Bio-Sciences AB, Sweden) column to purify the sample from lipoproteins, other low molecular weight impurities, and protamine.

In other embodiments, extracellular vesicles are isolated by neutralizing their surface charge with sodium acetate. Sodium acetate is thought to interfere with the hydration of extracellular vesicle surface, compensates the negative charge, and initiates extracellular vesicle aggregation via hydrophobic interactions. For example, the sample is first centrifuged (500×g, 30 min; 12,000×g, 30 min) to remove cells, debris, and large vesicles; then the supernatant is mixed with 0.1 volume of sodium acetate buffer (1.0 M pH 4.75) and incubated on ice for 30-60 min and additionally for 5 min at 37° C. Extracellular vesicles are sedimented by centrifugation (5000×g, 10 min); the pellet is washed with 0.1 M sodium acetate buffer and centrifuged under the same conditions to suspend the pellet in HBS (HEPES buffered saline). The precipitation procedure is repeated if necessary.

In other embodiments, extracellular vesicles are isolated based on precipitation of proteins with an organic solvent, PROSPR (PRotein Organic Solvent PRecipitation) rather than extracellular vesicle precipitation. This method is based on protein precipitation in acetone under conditions that retain hydrophobic vesicles in supernatant. For example, the sample is supplemented with fourfold volume of cold acetone (−20° C.) and centrifuged (3000×g for 1 min) and the supernatant containing extracellular vesicle fraction is concentrated in a vacuum concentrator.

In other embodiments, extracellular vesicles can be affinity purified. Because extracellular vesicles are rich in proteins and contain many receptors on their surfaces, antibodies can be used to purify them. For example, exosomes can be purified using antibodies specific for some of the most common exosomal protein markers, such as: CD9, CD81, CD63, CD82, Hsp70, Ras-related protein Rab-5b, cytoskeletal protein actin and TSG101. In particularly preferred embodiments, a microtubule associated protein (MAP) binding agent can be used to selectively bind extracellular vesicles expressing MAP and enrich for neuron derived extracellular vesicles.

Specific antibodies to extracellular vesicle markers can be used to select desired extracellular vesicle population (immunoenrichment) or to trap unwanted extracellular vesicle populations (negative selection or immunodepletion). Because extracellular vesicles are very heterogeneous in accordance to their origin, abundance of these markers on different extracellular vesicle also varies. So, a combination of specific antibodies can be used to capture different types of extracellular vesicles.

Antibodies covalently bound to the fixed phase may be used for this purpose. Magnetic beads, highly porous monolithic silica microtips, surface of plastic plates, cellulose filters, and membrane affinity filters are also used for this purpose.

In some embodiments, annexin 5, a protein binding to phosphatidylserine in the presence of calcium ions can be used to isolate extracellular vesicle having phosphatidylserine on their surface. Phosphatidylserine can be exposed on the surface of extracellular vesicles, in particular, microvesicles, apoptotic bodies, and, to a less degree, exosomes.

In some embodiments, extracellular vesicles are isolated based on the ability of heparin to bind extracellular vesicles. For example, extracellular vesicles can be isolated from conditioned cell culture medium using an agarose sorbent with heparin, e.g., Affi-Gel® Heparin Gel (Bio-Rad). Binding of heat shock proteins can also be used to isolate extracellular vesicles according to known methods. For example, the peptide venceremin (Vn) can be used.

In other embodiments, lectins can be used to agglutinate extracellular vesicles. Lectins are the proteins that reversibly, noncovalently, and highly specifically bind carbohydrate motifs of glycoproteins, proteoglycans, and glycolipids.

The skilled person would appreciate that the properties of a sample need to be taken into account when using a particular method for isolation of extracellular vesicles, since the protocol should be fit to specific characteristics of the sample, such as viscosity (when analysing the blood plasma and serum), presence of specific proteins (e.g., THP in the urine), extracellular concentration, and the type of further analysis/use of the isolated extracellular vesicles. It is well known that different methods can result in different extracellular vesicle subpopulations. Moreover, the extracellular vesicle isolation efficiency by different methods depends on the nature of biological fluids.

The isolated extracellular vesicles can be resuspended in any suitable buffer or medium for administration to an animal or for use in in vitro drug screening assays at any suitable protein concentration. For example, exosomes used for intranasal delivery can be resuspended in HEPES buffer at a protein concentration of about 2 mg/mL.

Once isolated, the extracellular vesicle preparation can be characterized of one or more of TEM, NTA, dynamic light scattering, flow cytometry, and antibodies used for markers specific of an isolated EV type can be used to characterise the extracellular vesicle morphology, biochemical composition, and the receptors expressed by the vesicles.

### Enriching for Neuron Derived Extracellular Vesicles

In some embodiments of the disclosure the methods involve preparing an extracellular vesicle fraction enriched for neuron derived extracellular vesicles. Such methods rely on immunoaffinity capture-based techniques.

Immunoaffinity capture-based techniques rely on the use of a binding agent, for example, an antibody or binding fragment thereof, to capture extracellular vesicles based on the expression of the antigen on the surface of the extracellular vesicle. Binding agents for a specific antigen of interest can be immobilized or conjugated onto/into a variety of solid media, such as magnetic beads or polymeric materials (e.g., agarose beads and monolithic columns). Advantageously, these methods allow for isolation of exosomes derived from a specific source, for example, from neurons.

In some examples, binding agents, for example, antibodies or binding fragments thereof are non-covalently coupled to magnetic beads. In other embodiments, the binding agents are covalently coupled to magnetic beads via, for example, biotinylation. Typically, bound extracellular vesicles are either eluted from the beads using appropriate reagents, or alternatively, bead-extracellular vesicle complexes can be directly used for further analysis.

A microfluidic device utilising immunocapture based isolation methods may be used and, in some embodiments, the device may be used for continuous extracellular vesicle isolation and detection.

Advantageously, such microfluidic techniques can be used to isolate extracellular vesicles based on their physical and biochemical properties simultaneously. Additionally, microfluidic isolation methods typically are rapid, efficient, require small starting volumes.

In some embodiments, immunoaffinity chromatography with monolithic materials may be used to isolate extracellular vesicles. Monolithic columns have several advantages over particulate-based stationary supports for the separation of large biomolecules, including low back pressure and convective mass transfer rather than diffusive transport, enabling high flow rates and short separation times due to large, interconnected pores. In particular, polymer-based monoliths can withstand alkaline pH conditions required for elution or desorption of bound particles unlike silica-based particles and monoliths. The polymer-based monoliths can also be directly coupled to other separation methods or detection systems for further analyses, making them attractive solid supports for extracellular vesicle isolation.

Besides magnetic beads and monolithic supports, other solid media may be used for extracellular vesicle isolation, such as magnetic nanowires, paper (cellulose), and nanospring.

One or more binding agents can be used for immunocapture of the extracellular vesicles. In some embodiments a microtubule associated protein (MAP) binding agent is used to selectively bind extracellular vesicles expressing MAP and enrich for neuron derived extracellular vesicles. The MAP binding agent may be used in combination with one or more additional binding agents, for example, against common markers such as CD9, CD81 and CD63 for immunocapture.

The present inventors have found that serum pre-clearing has no significant effect on yield when MAP binding agents are used. As such, a pre-clearing step is not needed. This advantageously minimizes technical variability and/or increases the number of samples that may be processed. Microtubule associated proteins are a diverse class that include motor, structural and enzymatic proteins. Microtubule associated protein 1B (MAP1B) is considered a classical structural MAP and belongs to the MAP1 family, as does MAP1A and MAP1S. The MAP2/tau family (MAP2, MAPT and MAP4) are also considered classical structural MAP's, yet they are otherwise unrelated to the MAP1 family.

The MAP1 family proteins are brain and neuron expressed; MAP1B and MAP1A show biased tissue expression for the brain whereas MAP1S is ubiquitously expressed. The three MAP1 proteins are encoded as polyproteins, each cleaved into a heavy chain and light chain where the light chains arise from the carboxyl terminus.

Overall, MAP1B and MAP1A share approximately 69% sequence identity, while MAP1B and MAP1S share approximately 43% sequence identity. Table 1 describes the results for non-intersecting local alignment between MAP1B and MAP1A followed by MAP1B and MAP1S, in order of decreasing similarity based on alignment score. Shared sequence identity between MAP1B and MAP1A is greatest at the N terminus (≈55%) followed by C terminus (≈50%). Shared sequence identity between MAP1B and MAP1S is greatest at the N terminus (≈43%) followed by C terminus (≈51%). The immunogenic sequence recognised by anti-MAP1B (residues 900-950) and the transmembrane domain (residues 790-810), are regions of interest with respect to the patent application. As shown in Table 1, sequence similarity and alignment scores between MAP1B and MAP1A are much lower in fragments covering these regions than in the N terminus and the C terminus. There was no reported sequence similarity between MAP1B and MAP1S for the regions of interest.

TABLE 1
Non-overlapping local alignment of MAP1 family proteins

| | | | Similarity | |
|---|---|---|---|---|
| | Residues | Region | (%) | Score |
| MAP1A | MAP1B: 223-726 | NH₂ | 55.4 | 1367.0 |
| | MAP1A: 1-497 | | | |
| | MAP1B: 2096-2467 | COOH | 50.1 | 692.0 |
| | MAP1A: 2447-2802 | | | |
| | MAP1B: 598-1345 | transmembrane | 23.7 | 233.0 |
| | MAP1A: 295-1019 | domain and | | |
| | MAP1B: 626-960 | immunogenic | 24.6 | 143.0 |
| | MAP1A: 329-670 | sequence | | |
| | MAP1B: 671-1172 | | 24.3 | 143.0 |
| | MAP1A: 314-823 | | | |
| MAP1S | MAP1B: 39-623 | NH₂ | 43.2 | 994.0 |
| | MAP1S: 17-561 | | | |
| | MAP1B: 2310-2467 | COOH | 51.2 | 373.0 |
| | MAP1S: 900-1058 | | | |
| | MAP1B: 1571-1596 | other | 80.8 | 100.0 |
| | MAP1S: 679-705 | | | |

Transmembrane domain (MAP1B residues 790-810).
Immunogenic sequence (MAP1B residues 900-950).

MAP binding agents useful in the methods of the disclosure include antibodies or binding fragments thereof such as, for example, rabbit, polyclonal, anti-MAP1B, catalogue #A301-446A, supplied by Bethyl laboratories.

The bound vesicles can be eluted with a low pH buffer. Following elution, the vesicles can be lysed with, for example, QIAzol/Trizol; the RNA extracted by addition of, for example, chloroform; and precipitated by, for example, addition of ethanol. In further embodiments, the vesicles can be further purified using, for example, a kit for RNA extraction from vesicles such as, for example, exoRNeasy from Qiagen.

### Diagnosis Based on Small Non-Coding RNA

In some embodiments, methods comprise identifying one or more small non-coding RNAs, for example, miRNAs indicative of a disease or condition or stage and comparing the level of particular non-coding RNAs and a level known to be indicative of an abnormal state. In some embodiments, said level has been correlated with a diagnosis; that is, the skilled person can use the level to determine whether the subject has a disease or condition or a pre-disposition thereto and respond accordingly. Alternatively, the level of particular non-coding RNAs can be compared to a level indicative of a normal state. In some embodiments, said level has been correlated with the absence of disease or a condition.

In some embodiments, methods for analysing a sample from a subject for a disease or condition comprises generating a small non-coding RNA profile (e.g., miRNA profile) for the sample and evaluating the profile to determine whether small non-coding RNA(s) in the sample are differentially expressed compared to a reference level.

In some embodiments, the reference level may be the average of a set of controls because a given control individual may be variable.

In other embodiments, the reference level may be the level of one or more small non-coding RNAs from a sample taken from the same subject at an earlier time point.

In some embodiments, to account for variability in sample input, the read count of one or more small non-coding RNAs is used as a proxy of molecules per mL of sample.

Three main techniques have been used to detect and quantify small non-coding RNAs (including miRNAs-cloning, hybridization with selective probes, and polymerase chain reaction (PCR)-based detection. Hybridization techniques include Northern blotting, bead-based flow-cytometry, and oligonucleotide microchip (microarray). Several commercially available small non coding RNA measurement platforms are available including microarray based and quantitative polymerase chain reaction (qPCR) based methods. The general detection principles for these methods are well known in the field. Microarray-based method usually involves the spotting or synthesizing RNA specific probe sequences on a solid support. The array is then hybridized with florescent or color matrix dye-labeled small non-coding RNAs from biological samples. The small non-coding RNA levels are then determined by the intensity of fluorescent or coloured dyes.

The comparison may involve using an array that has selective small non-coding RNA probes that are indicative of a disease or condition. Arrays include macroarrays and microarrays. Typically, arrays are made with materials that do not interfere with the hybridization between the probe and a sample. In some embodiments, the array is a solid support that is made with glass, plastic, or metal.

Clearly contemplated is an array that is a microarray. The arrays have one or more probes directed to one or more small non coding RNA (e.g., miRNA) molecules (to provide for example, a “miRNA array”).

An miRNA array includes one or more miRNA probes immobilized on a solid support. An “miRNA probe” refers to a nucleic acid having a sequence that is complementary or identical to all or part of a miRNA precursor or gene such that it is capable of specifically hybridizing to an miRNA gene, the cognate miRNA precursor, or the processed (mature) miRNA. Typically, the probe will contain at least ten contiguous nucleotides complementary to all or part of the miRNA precursor or at least ten contiguous nucleotides complementary or identical to all or part of an miRNA gene. It will be understood that DNA probes with sequences relative to an miRNA gene will be identical in sequence to all or part of the coding sequence of the gene and complementary in sequence to all or part of the noncoding sequence of the gene. In specific embodiments, an miRNA probe contains the sequence encoding an miRNA (“miRNA coding sequence” which refers to sequence encoding processed (mature) miRNA) or part thereof (e.g., seed sequence). Because the precise length and, consequently, sequence of a particular processed miRNA has been found to vary occasionally, the predominant species will be understood as the sequence and length of the processed miRNA. The predominant species is usually the one observed at least 90% of the time.

Arrays can contain small non-coding sequences from any organism having small non-coding sequences, specifically including, mammals such as humans. However, unless specifically indicated, the naming of a particular miRNA refers to a human miRNA. Specifically contemplated are arrays having, having at least, or having at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 5100, 5200, 5300, 5400, 5500, 5600, 5700, 5800, 5900, 6000, 6100, 6200, 6300, 6400, 6500, 6600, 6700, 6800, 6900, 7000, 7100, 7200, 7300, 7400, 7500, 7600, 7700, 7800, 7900, 8000, 8100, 8200, 8300, 8400, 8500, 8600, 8700, 8800, 8900, 9000, 9100, 9200, 9300, 9400, 9500, 9600, 9700, 9800, 9900, 10000 or more different probes (e.g., miRNA probes having different sequences targeting the same or different miRNAs, miRNA precursors, or miRNA genes).

In some embodiments, an amine is attached to the 5′ or 3′ end of the probe. The amine is a reactive group on the probe that allows for attachment of the probe to the array.

Specifically contemplated are arrays with probes for any of SEQ ID NOS: 1-29. Probes may be identical or complementary to 5, 6, 7, 8, 9, 10, 11, 12, 13, 14, 15, 16, 17, 18, 19, 20, 21, 22, 23, 24, 25, 26, 27, 28, 29 or more contiguous nucleic acids (or any range derivable therein) of SEQ ID NOs: 1-29. Alternatively, any probe used may be, be at least, or be at most 80, 81, 82, 83, 84, 85, 86, 87, 88, 89, 90, 91, 92, 93, 94, 95, 96, 97, 98, 99, or 100% complementary or identical (or any range derivable therein) to any sequence in SEQ ID NOs: 1-29. It is specifically contemplated that any embodiment discussed in the context of an array may be employed more generally in screening or profiling methods or kits of the invention. In other words, any embodiments describing what may be included in a particular array can be practiced in the context of profiling more generally and need not involve an array per se.

In specific embodiments, an array has, has at least, or has at most 1, 2, 3, 4, 5, 6, 7, 8, 9, 10, 11, 12, 13, 14, 15, 16, 17, 18, 19, 20, 21, 22, 23, 24, 25, 26, 27, 28, 29 probes (or any range derivable therein) for human miRNA selected from the group consisting of hsa-miR-1246, hsa-miR-486-5p, hsa-miR-5100, hsa-miR-3178, hsa-miR-7704, hsa-miR-203a-3p, hsa-miR-4521, hsa-miR-451a, hsa-miR-92a-3p, hsa-miR-484, hsa-miR-93-5p, hsa-miR-12136, hsa-miR-324-5p, hsa-miR-26a-5p, hsa-miR-146b-5p, hsa-let-7d-3p, hsa-miR-425-5p, hsa-miR-199a-5p, has-miR-590-5p, hsa-miR-126-3p, hsa-miR-10b-5p, hsa-miR-3648, hsa-miR-1843, hsa-miR-16-5p, hsa-miR-19b-3p, hsa-miR-23a-3p, hsa-miR-20a-5p, hsa-miR-21-5p, and hsa-let-7a-5p. It will be understood that shorthand notations are employed such that a generic description of a miRNA refers to any of its gene family members (distinguished by a number) and related members (distinguished by a letter), unless otherwise indicated. Exceptions to this shorthand notations will be otherwise identified. A probe with at least 90% complementarity will allow hybridization to a miRNA. It is contemplated that a probe for a non-human miRNA can be used in embodiments of the invention to target human homologs or sequences with sufficient complementarity to allow their detection with the non-human miRNA probe. Such probes may be for miRNA identified in non-human subjects.

It is contemplated that any combination of these probes or the target miRNAs can be used in methods and compositions of the invention. Furthermore, an increase and/or decrease in expression of, of at least, or of at most 1, 2, 3, 4, 5, 6, 7, 8, 9, 10, 11, 12, 13, 14, 15, 16, 17, 18, 19, 20, 21, 22, 23, 24, 25, 26, 27, 28, 29 or more of these miRNAs relative to a reference level.

Direct hybridization of miRNA samples onto a microarray may require a large amount of total RNA. With small or limited RNA samples, a PCR based approach can be used.

In this method, total RNA is isolated as usual. However, a reverse transcriptase (RT) reaction follows. The RT reaction first consists of small RNA fractionation, followed by polyadenylation. Then a standard RT protocol is applied where poly(T)s are added to prime the synthesized the poly(A) tail so reverse transcriptase can produce cDNAs from the small RNA. Finally, small non-coding RNA (e.g., miRNA) specific primers will probe for a specific small non-coding RNA (e.g., miRNA) through PCR amplification. Due to specificity issues and inability to differentiate between mature and pre-miRNA, changes can be made to the RT step. Instead of a general poly(A) reaction in combination with universal priming through poly(T) adapter molecules, a miRNA specific stem-loop reverse primer can be used. This specially designed primer contains sequence that is antisense to a portion of the 3′ sequence of the miRNA that is to be amplified. To increase the specificity of the PCR amplification step, the forward primer contains antisense sequence derived from the mature miRNA, and the reverse primer consists of sequences taken from the stem-loop of the reverse primer. In some embodiments, qPCR based systems are used to detect and quantify one or more small non-coding RNAs in a sample.

The qPCR based system generally involves modification of the miRNA templates so that they are long enough to support two independent primers that are required for PCR.

In one system, marketed as TaqMan® by Life Technologies, a hairpin nucleic acid molecule with one prong shortened and the other extended to be complementary to the 3′ portion of the targeted miRNA is first annealed to the miRNA in the sample. The longer arm of the hairpin is then further extended by using the miRNA as a template to generate miRNA cDNA. The loop is then opened to serve as an adaptor for extension of the original microRNA thus resulting in a longer RNA molecule for amplification. The amplified molecule is detected with a probe containing a fluorescence quenching system.

In another system, marketed by Qiagen, a polyA tail is added to the miRNA. The miRNA is then transcribed into cDNA by annealing a polyT with a tag sequence to the polyA. The resulting cDNA can then be used in detecting specific miRNA by PCR using an miRNA specific primer and a polyA primer with a tag sequence at 3′end. The miRNA specific primer usually covers the entire length of the miRNA because of the short sequence of the miRNA.

An additional system, Exiqon, uses a similar approach to that of Qiagen but with different tag sequence. It incorporates modified nucleotides into the primer to provide “locked” nucleic acid, to increase primer specificity.

Advantageously, PCR-based techniques are able to detect low copy numbers of individual miRNAs with high sensitivity and specificity on both the precursor and the mature form of miRNAs. The stem-loop reverse transcription-polymerase chain reaction (RT-PCR) can profile miRNA expression with only nanograms of total RNA or even single cells. qPCR combines the high sensitivity provided by the stem-loop RT-PCR with the ability to profile large numbers of miRNA in a single experiment.

In other embodiments, RNA-seq is used to detect and quantify one or more small non-coding RNAs in a sample.

An RNA-seq workflow has several steps, which can be broadly summarized as: RNA extraction, Reverse transcription into cDNA, Adapted ligation, Amplification and Sequencing.

Once the RNA sample has been obtained, the first step in the technique involves converting the population of RNA to be sequenced into complimentary DNA (cDNA) fragments (a cDNA library). This is done by reverse transcription and allows the RNA to be put into an NGS workflow. The cDNA may then be fragmented, and adapters are added to each end of the fragments. These adapters contain functional elements which permit sequencing, for example, the amplification element (which facilitates clonal amplification of the fragments) and the primary sequencing priming site. In small RNA-sequence workflow, the cDNA does not need to be fragmented before adding the adapters. Following processes of amplification, size selection, clean-up and quality checking, the cDNA library is then analyzed by NGS, producing short sequences that correspond to all or part of the fragment from which it was derived. The depth to which the library is sequenced varies depending on the purpose for which the output data will be used for. Sequencing may follow either single-end or paired-end sequencing methods. Single-read sequencing is a cheaper and faster technique (for reference, about 1% of the cost of Sanger sequencing) that sequences the cDNA fragments from just one end. Paired-end methods sequence from both ends and are therefore more expensive but offer advantages in post-sequencing data reconstruction.

Advantageously, RNA-seq is not limited to genomic sequences-unlike hybridization-based approaches, which may require species-specific probes, RNA-seq can detect transcripts with previously undetermined genomic sequences. Further, it has a low background signal—the cDNA sequences used in RNA-seq can be mapped to targeted regions on the genome, which makes it easy to remove experimental noise. It is quantifiable-microarray data for example is displayed as values relative to other signals detected on the array, whilst RNA-seq data is quantifiable. While it cannot quantify miRNA levels with the molar resolution of qPCR, deep sequencing of miRNA does have the advantage of being able to sample all miRNAs present in a sample, whether the sequence is known or not. Furthermore, as sequences are read directly, RNA-seq can distinguish closely related miRNAs and isoforms.

In some embodiments, the level of the one or more small non-coding RNA is combined with one or more clinical parameters selected from the group consisting of age, gender, Diagnostic Interview for Psychosis (DIP) score, substance use disorder, IQ, neuropsychological status, working memory and executive functioning for diagnosis. Subjects may be excluded on the basis of organic brain disorder, brain injury with >24 hours post-event amnesia, IQ<70, movement disorders, current diagnosis of substance dependence and electroconvulsive therapy within last 6 months.

The term “combined” or variations such as “combination” or “combining” is defined as a possible selection of a certain number of parameters and the arrangement of these parameters into specified groups using a mathematical algorithm (e.g., deviation or ratio). For example, a ratio can be calculated between the level of a biomarker (e.g., miRNA) in a sample taken from a patient and the level of the same biomarker in a sample taken from a reference sample, or the patient at baseline. Moreover, a deviation can be calculated between the level of a biomarker in a sample taken from a patient and the level of the same biomarker in a reference sample, or the patient at baseline. It is also encompassed herein, that a ratio between different biomarkers can be calculated. For example, the ratio can be calculated between biomarker levels measured in samples taken from the patient at the same time point or at different time points.

In some embodiments, the level of the at least one biomarker can be combined as continuous or categorical variable.

The term “score” in the context of the present disclosure refers to a rating, expressed numerically, based on the specific achievement or the degree to which certain qualities or conditions (e.g. the level of one or more small non-coding RNAs) are present in the sample.

In some embodiments of the disclosure, the level of one or more small non-coding RNAs in the sample can be used to stage the severity of the disease or condition. For example, the level of one or more small non-coding RNAs in the sample can be used to stage the severity of disease state.

In some embodiments of the disclosure, the subject is stratified as having early, moderate or severe neurodegenerative disease or neuropsychiatric disease.

The term “treatment stratification” in the context of the present disclosure refers to the choice and/or adjustment of a therapeutic treatment of the subject. For example, subjects having a log₂ fold decrease of about 1.356 [95% CI: −2.19, −0.52] in the level of miR-199a-5p may be responsive to treatment, for example, to clozapine intervention.

The term “risk stratification” in the context of the present disclosure refers to a probability of specified outcomes for a subject. For example, subjects having about a log₂ fold change (95% confidence level) in one or more small non-coding RNAs may be at risk for disease state (see, for example Table 7). For example, a log₂ fold decrease of about 1.278 [95% CI: −1.87, −0.68] in the level of mir-451a or a log₂ fold increase of 1.278 [95% CI: 0.83, 1.72] in the level of mir-1246 may indicate risk for disease state.

### EXAMPLES

### Example 1

### Materials and Methods

### Participants and Study Design

Participants were drawn from the Australian Schizophrenia Research Bank (ASRB), a national research repository of phenotypic data and biological samples from English speaking people aged 18-65 years. Clinical assessments at the time of ASRB enrolment included the Diagnostic Interview for Psychosis (DIP), substance use disorder, IQ, neuropsychological status, working memory and executive functioning. Participants were excluded on the basis of organic brain disorder, brain injury with >24 hours post-event amnesia, IQ<70, movement disorders, current diagnosis of substance dependence and electroconvulsive therapy within last 6 months. Additionally for controls, a personal or family history of psychosis or bipolar type 1 disorder led to exclusion (Loughland et al., 2010). The current work includes 221 participants with lifetime diagnosis of a psychotic disorder and 256 non-psychiatric controls. A summary of demographic and clinical characteristics for the cohort are shown in Table 2. Refer to Table 3 for a full description of cohort demographic and clinical characteristics.

Clustering of subjects by cognitive performance is described by Green et al. (Green et al., 2013). Briefly, Grade of Membership (GoM) was used to determine membership of schizophrenia subjects based on input variables spanning 9 cognitive measures. The most parsimonious model included 2 classes of membership; cognitively spared subjects had low probabilities of cognitive impairment while cognitive deficit subjects showed higher probabilities of poor performance on all cognitive measures.

TABLE 2
Summary of clinical and demographic characteristics
for schizophrenia and nonpsychiatric control cohort.

| | | Summary |
|---|---|---|
| Demographic and clinical variables | | statistics |
| Diagnosis | schizophrenia/control | 221/256 |
| Cognitive subtype | spared/deficit | 110/111 |
| Mean age | schizophrenia/spared/deficit | 40.0/40.5/39.4 |
| | control | 44.2 |
| Gender (male/female) | schizophrenia | 147/74 |
| | control | 111/145 |
| Mean age at illness onset (male/female) | schizophrenia | 23.8/26.1 |
| Mean age at illness onset | | 24.9/24.2 |
| (spared/deficit) | | |
| Mean duration of illness (male/female) | | 15.1/16.0 |
| Mean duration of illness (spared/deficit) | | 15.6/15.2 |
| Number of subjects using antipsychotic | | 200 |
| medications | | |
| Number of treatment resistant subjects | | 42 |
| (taking clozapine) | | |
| Number of subjects using | | 60 |
| antidepressant medication | | |
| Number of early onset subjects (onset | | 31 |
| before 18 years age) | | |

TABLE 3
Detailed demographic and clinical variables of cohort.

| | | | | | | Family history | | | |
|---|---|---|---|---|---|---|---|---|---|
| | | | | | | (schizophrenia | Family history | | Medication |
| | | Cognitive | | | Age at | or other | (other mental | Duration of | (antipsychotic, |
| # | Diagnosis | subtype | Age | Gender | onset | psychosis) | condition) | illness (years) | antidepressant) |
| 1 | CTRL | N/A | 46 | F | N/A | absent | present | N/A | N/A |
| 2 | CTRL | N/A | 31 | F | N/A | absent | absent | N/A | N/A |
| 3 | SZ | CD | 39 | M | 16 | absent | present | 2 | Aripiprazole |
| 4 | SZ | CD | 37 | M | 17 | present | present | 3 | Clozapine |
| 5 | CTRL | N/A | 44 | F | N/A | absent | absent | N/A | N/A |
| 6 | SZ | CS | 24 | M | 19 | absent | absent | 3 | Risperidone, |
| | | | | | | | | | Sertraline |
| 7 | SZ | CD | 21 | F | 13 | absent | present | 2 | Olanzapine, |
| | | | | | | | | | Quetiapine |
| | | | | | | | | | fumerate, |
| | | | | | | | | | Risperidone, |
| | | | | | | | | | Thioridazine |
| | | | | | | | | | hydrochloride, |
| | | | | | | | | | Venlafaxine |
| 8 | CTRL | N/A | 55 | M | N/A | absent | present | N/A | N/A |
| 9 | CTRL | N/A | 25 | M | N/A | absent | present | N/A | N/A |
| 10 | SZ | CS | 35 | M | 24 | absent | present | 2 | Amisulpride, |
| | | | | | | | | | Aripiprazole, |
| | | | | | | | | | Escitalopram, |
| | | | | | | | | | Venlafaxine |
| 11 | SZ | CD | 20 | M | 16 | absent | present | 2 | Amisulpride, |
| | | | | | | | | | Chlorpromazine, |
| | | | | | | | | | Clozapine, |
| | | | | | | | | | Venlafaxine |
| 12 | SZ | CS | 65 | M | 27 | absent | absent | 2 | Olanzapine |
| 13 | CTRL | N/A | 30 | M | N/A | absent | present | N/A | N/A |
| 14 | SZ | CS | 64 | M | 19 | present | absent | 3 | N/A |
| 15 | SZ | CS | 54 | F | 20 | present | present | 2 | N/A |
| 16 | CTRL | N/A | 29 | F | N/A | absent | present | N/A | N/A |
| 17 | SZ | CD | 44 | M | 27 | absent | absent | 3 | Aripiprazole, |
| | | | | | | | | | Quetiapine |
| | | | | | | | | | fumerate |
| 18 | SZ | CD | 43 | M | 26 | absent | absent | 2 | Amisulpride, |
| | | | | | | | | | Ziprasidone |
| | | | | | | | | | hydrochloride |
| 19 | SZ | CS | 57 | M | 17 | absent | absent | 3 | Fluphenazine |
| | | | | | | | | | decanoate |
| 20 | SZ | CD | 41 | M | 33 | present | present | 3 | Haloperidol |
| | | | | | | | | | decanoate |
| 21 | CTRL | N/A | 61 | F | N/A | absent | absent | N/A | N/A |
| 22 | CTRL | N/A | 35 | F | N/A | absent | absent | N/A | N/A |
| 23 | CTRL | N/A | 30 | M | N/A | absent | absent | N/A | N/A |
| 24 | CTRL | N/A | 41 | M | N/A | absent | present | N/A | N/A |
| 25 | SZ | CS | 42 | F | 35 | present | present | 3 | Quetiapine |
| | | | | | | | | | fumerate, |
| | | | | | | | | | Tranylcypromine |
| 26 | SZ | CS | 61 | M | 14 | present | present | 2 | Olanzapine |
| 27 | SZ | CD | 28 | M | 15 | present | present | 2 | Olanzapine |
| 28 | CTRL | N/A | 21 | F | N/A | absent | present | N/A | N/A |
| 29 | SZ | CD | 30 | F | 24 | present | absent | 3 | Amisulpride, |
| | | | | | | | | | Aripiprazole |
| 30 | SZ | CD | 24 | M | 11 | absent | present | 2 | Amisulpride, |
| | | | | | | | | | Clozapine, |
| | | | | | | | | | Paroxetine |
| 31 | CTRL | N/A | 27 | F | N/A | absent | present | N/A | N/A |
| 32 | SZ | CD | 35 | M | 22 | present | present | 2 | Quetiapine |
| | | | | | | | | | fumerate |
| 33 | CTRL | N/A | 29 | M | N/A | absent | absent | N/A | N/A |
| 34 | SZ | CD | 62 | M | 41 | absent | present | 3 | Quetiapine |
| | | | | | | | | | fumerate |
| 35 | SZ | CS | 47 | M | 35 | absent | present | 2 | Risperidone |
| 36 | SZ | CS | 36 | F | 33 | present | present | 2 | Olanzapine, |
| | | | | | | | | | Risperidone |
| 37 | CTRL | N/A | 44 | F | N/A | absent | present | N/A | N/A |
| 38 | CTRL | N/A | 37 | F | N/A | absent | present | N/A | N/A |
| 39 | CTRL | N/A | 30 | M | N/A | absent | absent | N/A | N/A |
| 40 | SZ | CS | 54 | F | 21 | present | present | 2 | Amisulpride |
| 41 | SZ | CS | 62 | F | 54 | absent | present | 3 | Risperidone |
| 42 | CTRL | N/A | 48 | F | N/A | absent | present | N/A | N/A |
| 43 | CTRL | N/A | 28 | M | N/A | absent | present | N/A | N/A |
| 44 | SZ | CD | 38 | M | 29 | absent | absent | 2 | Aripiprazole |
| 45 | SZ | CS | 26 | F | 23 | absent | present | 2 | Aripiprazole, |
| | | | | | | | | | Venlafaxine |
| 46 | SZ | CD | 29 | M | 17 | absent | absent | 2 | Amisulpride |
| 47 | CTRL | N/A | 23 | F | N/A | absent | absent | N/A | N/A |
| 48 | SZ | CS | 31 | M | 23 | present | absent | 2 | Quetiapine |
| | | | | | | | | | fumerate, |
| | | | | | | | | | Mirtazapine |
| 49 | SZ | CS | 20 | M | 16 | absent | present | 2 | N/A |
| 50 | SZ | CS | 40 | M | 27 | present | present | 3 | Amisulpride |
| 51 | CTRL | N/A | 50 | F | N/A | absent | present | N/A | N/A |
| 52 | CTRL | N/A | 19 | M | N/A | absent | present | N/A | N/A |
| 53 | SZ | CD | 40 | M | 25 | absent | present | 3 | Amisulpride, |
| | | | | | | | | | Risperidone |
| 54 | SZ | CD | 53 | F | 46 | absent | present | 3 | Amisulpride |
| 55 | SZ | CS | 51 | F | 26 | present | present | 3 | Amisulpride |
| 56 | SZ | CD | 36 | M | 31 | absent | present | 3 | Risperidone |
| 57 | SZ | CS | 33 | M | 24 | present | present | 3 | Quetiapine |
| | | | | | | | | | fumerate, |
| | | | | | | | | | Risperidone |
| 58 | SZ | CS | 31 | M | 22 | absent | present | 3 | Amisulpride |
| 59 | SZ | CS | 33 | F | 19 | absent | present | 3 | Aripiprazole, |
| | | | | | | | | | Quetiapine |
| | | | | | | | | | fumerate, |
| | | | | | | | | | Reboxetine |
| 60 | SZ | CD | 35 | M | 25 | absent | absent | 2 | Clozapine |
| 61 | SZ | CS | 60 | F | 21 | absent | present | 3 | Quetiapine |
| | | | | | | | | | fumerate |
| 62 | SZ | CS | 23 | F | 22 | absent | present | 2 | Risperidone, |
| | | | | | | | | | Citalopram |
| 63 | SZ | CS | 53 | F | 37 | present | absent | 3 | Olanzapine |
| 64 | SZ | CS | 50 | M | 19 | absent | present | 3 | Aripiprazole |
| 65 | SZ | CD | 31 | M | 16 | absent | present | 3 | Clozapine |
| 66 | SZ | CD | 35 | M | 22 | absent | present | 3 | Olanzapine, |
| | | | | | | | | | Venlafaxine |
| 67 | SZ | CS | 32 | M | 19 | absent | present | 2 | Trifluoperazine |
| | | | | | | | | | hydrochloride |
| 68 | SZ | CS | 53 | M | 21 | present | present | 3 | Olanzapine |
| 69 | SZ | CS | 63 | F | 28 | absent | present | 3 | Aripiprazole |
| 70 | SZ | CS | 53 | F | 22 | absent | present | 3 | Olanzapine, |
| | | | | | | | | | Risperidone, |
| | | | | | | | | | Citalopram |
| 71 | SZ | CS | 44 | M | 29 | present | present | 2 | Amisulpride |
| 72 | SZ | CS | 58 | M | 25 | absent | present | 2 | Olanzapine |
| 73 | SZ | CD | 45 | M | 23 | absent | absent | 2 | Clozapine |
| 74 | SZ | CS | 45 | F | 20 | absent | present | 3 | Mirtazapine |
| 75 | SZ | CD | 39 | M | 22 | present | present | 2 | Olanzapine |
| 76 | SZ | CD | 30 | M | 22 | absent | present | 2 | Amisulpride, |
| | | | | | | | | | Sertraline |
| 77 | CTRL | N/A | 36 | F | N/A | absent | present | N/A | N/A |
| 78 | SZ | CS | 28 | F | 22 | absent | present | 3 | Amisulpride, |
| | | | | | | | | | Ziprasidone |
| | | | | | | | | | hydrochloride |
| 79 | SZ | CS | 34 | F | 24 | absent | absent | 2 | Clozapine, |
| | | | | | | | | | Sertraline |
| 80 | SZ | CD | 26 | M | 17 | present | present | 2 | Quetiapine |
| | | | | | | | | | fumerate, |
| | | | | | | | | | Risperidone |
| 81 | CTRL | N/A | 45 | M | N/A | absent | present | N/A | N/A |
| 82 | SZ | CD | 55 | F | 31 | absent | present | 3 | Fluphenazine |
| | | | | | | | | | decanoate |
| 83 | SZ | CS | 50 | F | 21 | absent | present | 2 | Amisulpride |
| 84 | SZ | CS | 25 | M | 20 | absent | absent | 2 | Aripiprazole |
| 85 | CTRL | N/A | 26 | M | N/A | absent | absent | N/A | N/A |
| 86 | CTRL | N/A | 46 | F | N/A | absent | present | N/A | N/A |
| 87 | CTRL | N/A | 57 | M | N/A | absent | present | N/A | N/A |
| 88 | SZ | CD | 41 | M | 23 | present | present | 2 | Risperidone |
| 89 | CTRL | N/A | 27 | F | N/A | absent | present | N/A | N/A |
| 90 | CTRL | N/A | 23 | M | N/A | absent | present | N/A | N/A |
| 91 | SZ | CD | 31 | M | 17 | absent | present | 3 | Risperidone, |
| | | | | | | | | | dothiepin |
| 92 | SZ | CD | 37 | F | 21 | present | absent | 2 | Clozapine |
| 93 | SZ | CS | 41 | M | 21 | present | present | 2 | Risperidone, |
| | | | | | | | | | Reboxetine |
| 94 | SZ | CS | 45 | M | 26 | absent | present | 2 | Risperidone |
| 95 | CTRL | N/A | 37 | F | N/A | absent | present | N/A | N/A |
| 96 | SZ | CD | 43 | F | 30 | present | absent | 3 | Ziprasidone |
| | | | | | | | | | hydrochloride |
| 97 | SZ | CS | 52 | F | 30 | present | present | 3 | Aripiprazole |
| 98 | SZ | CS | 45 | M | 43 | absent | present | 3 | N/A |
| 99 | CTRL | N/A | 24 | F | N/A | absent | present | N/A | N/A |
| 100 | CTRL | N/A | 38 | M | N/A | absent | absent | N/A | N/A |
| 101 | CTRL | N/A | 22 | F | N/A | absent | absent | N/A | N/A |
| 102 | CTRL | N/A | 48 | M | N/A | absent | absent | N/A | N/A |
| 103 | CTRL | N/A | 47 | M | N/A | absent | present | N/A | N/A |
| 104 | CTRL | N/A | 32 | M | N/A | absent | absent | N/A | N/A |
| 105 | SZ | CD | 57 | M | 27 | absent | absent | 3 | Clozapine, |
| | | | | | | | | | Fluoxetine |
| 106 | CTRL | N/A | 22 | F | N/A | absent | present | N/A | N/A |
| 107 | SZ | CS | 32 | M | 23 | absent | present | 2 | N/A |
| 108 | SZ | CD | 33 | M | 21 | absent | present | 2 | Clozapine |
| 109 | SZ | CD | 55 | F | 30 | absent | present | 3 | Quetiapine |
| | | | | | | | | | fumerate, |
| | | | | | | | | | Risperidone, |
| | | | | | | | | | Sertraline |
| 110 | SZ | CD | 40 | F | 22 | present | present | 3 | Quetiapine |
| | | | | | | | | | fumerate, |
| | | | | | | | | | Zuclopenthixol, |
| | | | | | | | | | Fluoxetine |
| 111 | SZ | CD | 36 | F | 26 | absent | present | 3 | Clozapine |
| 112 | CTRL | N/A | 50 | M | N/A | absent | present | N/A | N/A |
| 113 | SZ | CS | 27 | F | 20 | present | present | 3 | Risperidone |
| 114 | SZ | CS | 36 | F | 16 | present | present | 2 | Amisulpride, |
| | | | | | | | | | Clozapine |
| 115 | SZ | CS | 47 | F | 26 | present | present | 2 | Aripiprazole, |
| | | | | | | | | | Quetiapine |
| | | | | | | | | | fumerate |
| 116 | CTRL | N/A | 40 | M | N/A | absent | present | N/A | N/A |
| 117 | SZ | CD | 28 | M | 17 | absent | absent | 2 | Amisulpride, |
| | | | | | | | | | Clozapine, |
| | | | | | | | | | Fluoxetine |
| 118 | SZ | CD | 52 | F | 21 | present | present | 3 | Fluoxetine |
| 119 | SZ | CS | 42 | M | 24 | present | absent | 2 | Olanzapine |
| 120 | SZ | CS | 37 | M | 26 | absent | present | 2 | Risperidone |
| 121 | SZ | CS | 26 | F | 13 | present | present | 3 | Quetiapine |
| | | | | | | | | | fumerate, |
| | | | | | | | | | Venlafaxine |
| 122 | SZ | CD | 50 | M | 20 | absent | absent | 2 | N/A |
| 123 | SZ | CD | 30 | F | 19 | absent | present | 3 | Aripiprazole, |
| | | | | | | | | | Quetiapine |
| | | | | | | | | | fumerate, |
| | | | | | | | | | Mirtazapine |
| 124 | SZ | CS | 36 | M | 23 | present | present | ~2 | Aripiprazole |
| 125 | CTRL | N/A | 46 | M | N/A | absent | present | N/A | N/A |
| 126 | SZ | CD | 43 | M | 22 | absent | present | 2 | Aripiprazole, |
| | | | | | | | | | Quetiapine |
| | | | | | | | | | fumerate, |
| | | | | | | | | | Sertraline |
| 127 | SZ | CS | 28 | M | 17 | absent | absent | 2 | Risperidone |
| 128 | SZ | CS | 33 | M | 19 | absent | absent | 2 | Clozapine |
| 129 | SZ | CD | 27 | F | 25 | present | present | 3 | Quetiapine |
| | | | | | | | | | fumerate |
| 130 | SZ | CS | 58 | M | 34 | absent | present | 2 | N/A |
| 131 | SZ | CD | 24 | M | 17 | absent | absent | 2 | Trifluoperazine |
| | | | | | | | | | hydrochloride |
| 132 | SZ | CS | 42 | M | 39 | present | present | 3 | Quetiapine |
| | | | | | | | | | fumerate |
| 133 | SZ | CD | 32 | M | 17 | absent | present | 3 | Olanzapine |
| 134 | CTRL | N/A | 48 | F | N/A | absent | present | N/A | N/A |
| 135 | SZ | CS | 52 | M | 32 | absent | present | 2 | Trifluoperazine |
| | | | | | | | | | hydrochloride |
| 136 | CTRL | N/A | 57 | M | N/A | absent | present | N/A | N/A |
| 137 | CTRL | N/A | 54 | M | N/A | absent | absent | N/A | N/A |
| 138 | CTRL | N/A | 36 | M | N/A | absent | present | N/A | N/A |
| 139 | CTRL | N/A | 24 | F | N/A | absent | present | N/A | N/A |
| 140 | CTRL | N/A | 48 | M | N/A | absent | present | N/A | N/A |
| 141 | SZ | CD | 37 | M | 17 | absent | present | 2 | Amisulpride, |
| | | | | | | | | | Quetiapine |
| | | | | | | | | | fumerate |
| 142 | SZ | CD | 40 | F | 24 | present | present | 3 | Ziprasidone |
| | | | | | | | | | hydrochloride |
| 143 | SZ | CD | 34 | M | 18 | absent | present | 2 | Quetiapine |
| | | | | | | | | | fumerate, |
| | | | | | | | | | Sertraline |
| 144 | SZ | CD | 41 | M | 29 | absent | present | 3 | Aripiprazole, |
| | | | | | | | | | Risperidone, |
| | | | | | | | | | Mirtazapine |
| 145 | SZ | CD | 52 | F | 40 | absent | present | 2 | Olanzapine |
| 146 | SZ | CS | 42 | F | 18 | absent | present | 2 | Olanzapine |
| 147 | SZ | CS | 39 | F | 28 | present | present | 2 | N/A |
| 148 | SZ | CS | 20 | F | 16 | absent | present | 2 | Aripiprazole |
| 149 | SZ | CS | 32 | M | 21 | absent | absent | 3 | Aripiprazole |
| 150 | SZ | CD | 40 | M | 34 | absent | absent | 3 | Amisulpride, |
| | | | | | | | | | Venlafaxine |
| 151 | SZ | CD | 34 | M | 20 | absent | present | 2 | Aripiprazole, |
| | | | | | | | | | Olanzapine, |
| | | | | | | | | | Mirtazapine |
| 152 | SZ | CS | 44 | F | 40 | absent | present | 3 | Aripiprazole, |
| | | | | | | | | | Sertraline |
| 153 | SZ | CD | 41 | M | 37 | absent | present | 3 | Olanzapine, |
| | | | | | | | | | Risperidone |
| 154 | SZ | CS | 39 | F | 28 | absent | absent | 2 | Amisulpride, |
| | | | | | | | | | Clozapine |
| 155 | CTRL | N/A | 23 | F | N/A | absent | absent | N/A | N/A |
| 156 | SZ | CD | 36 | M | 22 | present | present | 3 | Aripiprazole, |
| | | | | | | | | | Clozapine, |
| | | | | | | | | | Citalopram |
| 157 | CTRL | N/A | 43 | M | N/A | absent | present | N/A | N/A |
| 158 | SZ | CD | 23 | M | 21 | absent | absent | 2 | Risperidone |
| 159 | SZ | CS | 37 | M | 21 | absent | present | 2 | Clozapine |
| 160 | CTRL | N/A | 59 | F | N/A | absent | present | N/A | N/A |
| 161 | SZ | CS | 35 | F | 26 | absent | present | 3 | Aripiprazole, |
| | | | | | | | | | Escitalopram |
| 162 | SZ | CS | 45 | M | 34 | absent | absent | 3 | Olanzapine |
| 163 | SZ | CD | 32 | M | 27 | absent | present | 2 | Amisulpride, |
| | | | | | | | | | Flupenthixol |
| | | | | | | | | | decanoate |
| 164 | SZ | CD | 41 | M | 37 | absent | absent | 3 | Amisulpride, |
| | | | | | | | | | Aripiprazole |
| 165 | SZ | CD | 25 | M | 18 | absent | present | 2 | Clozapine, |
| | | | | | | | | | Risperidone, |
| | | | | | | | | | Sertraline |
| 166 | SZ | CD | 29 | M | 23 | present | absent | 2 | Risperidone |
| 167 | SZ | CS | 49 | M | 20 | absent | present | 2 | Olanzapine |
| 168 | SZ | CS | 37 | F | 24 | present | present | 2 | N/A |
| 169 | CTRL | N/A | 45 | M | N/A | absent | present | N/A | N/A |
| 170 | CTRL | N/A | 20 | F | N/A | absent | present | N/A | N/A |
| 171 | SZ | CD | 47 | F | 24 | absent | absent | 3 | Amisulpride |
| 172 | SZ | CS | 51 | M | 18 | absent | absent | 2 | N/A |
| 173 | SZ | CD | 30 | F | 21 | absent | present | 3 | Amisulpride |
| 174 | SZ | CS | 25 | F | 19 | absent | present | 2 | Amisulpride |
| 175 | SZ | CS | 44 | F | 14 | present | present | 3 | Clozapine, |
| | | | | | | | | | Risperidone, |
| | | | | | | | | | Venlafaxine |
| 176 | SZ | CS | 43 | M | 32 | absent | present | 3 | Risperidone |
| 177 | CTRL | N/A | 25 | F | N/A | absent | present | N/A | N/A |
| 178 | CTRL | N/A | 38 | F | N/A | absent | absent | N/A | N/A |
| 179 | SZ | CD | 54 | M | 38 | absent | present | 2 | Olanzapine, |
| | | | | | | | | | Zuclopenthixol |
| 180 | CTRL | N/A | 55 | F | N/A | absent | absent | N/A | N/A |
| 181 | CTRL | N/A | 48 | F | N/A | absent | absent | N/A | N/A |
| 182 | SZ | CS | 49 | M | 25 | absent | absent | 3 | Risperidone |
| 183 | SZ | CD | 27 | M | 23 | absent | absent | 3 | Olanzapine |
| 184 | SZ | CS | 55 | F | 44 | present | present | 3 | Risperidone |
| 185 | SZ | CS | 28 | M | 15 | present | present | 2 | Clozapine |
| 186 | SZ | CS | 33 | M | 30 | absent | present | 2 | Amisulpride |
| 187 | SZ | CD | 46 | F | 14 | absent | absent | 3 | Risperidone |
| 188 | SZ | CS | 55 | F | 25 | absent | present | 2 | Amisulpride |
| 189 | CTRL | N/A | 49 | F | N/A | absent | present | N/A | N/A |
| 190 | SZ | CD | 33 | M | 29 | present | present | 3 | Clozapine |
| 191 | SZ | CD | 34 | M | 21 | absent | present | 3 | Clozapine |
| 192 | SZ | CS | 25 | M | 19 | absent | present | 2 | Clozapine, |
| | | | | | | | | | Citalopram |
| 193 | CTRL | N/A | 24 | F | N/A | absent | absent | N/A | N/A |
| 194 | SZ | CD | 40 | M | 31 | present | present | 2 | Olanzapine, |
| | | | | | | | | | Citalopram |
| 195 | SZ | CS | 39 | M | 29 | present | present | 3 | Pericyazine, |
| | | | | | | | | | Citalopram |
| 196 | SZ | CD | 57 | F | 23 | absent | absent | 3 | Olanzapine |
| 197 | SZ | CD | 50 | M | 21 | absent | absent | 3 | Flupenthixol |
| | | | | | | | | | decanoate |
| 198 | SZ | CD | 46 | M | 31 | present | present | 2 | Amisulpride |
| 199 | SZ | CD | 39 | M | 24 | absent | absent | 2 | Flupenthixol |
| | | | | | | | | | decanoate |
| 200 | SZ | CS | 31 | M | 25 | present | present | 3 | Aripiprazole, |
| | | | | | | | | | Sertraline |
| 201 | SZ | CS | 36 | F | 20 | absent | present | 3 | Olanzapine, |
| | | | | | | | | | Sertraline |
| 202 | CTRL | N/A | 22 | M | N/A | absent | absent | N/A | N/A |
| 203 | CTRL | N/A | 20 | F | N/A | absent | present | N/A | N/A |
| 204 | SZ | CS | 35 | M | 34 | absent | present | 2 | Olanzapine |
| 205 | SZ | CD | 25 | M | 24 | absent | present | 2 | Risperidone |
| 206 | SZ | CD | 36 | M | 29 | absent | absent | 2 | Olanzapine |
| 207 | SZ | CS | 38 | F | 19 | absent | present | 3 | Quetiapine |
| | | | | | | | | | fumerate, |
| | | | | | | | | | Sertraline |
| 208 | CTRL | N/A | 34 | F | N/A | absent | present | N/A | N/A |
| 209 | CTRL | N/A | 35 | F | N/A | absent | present | N/A | N/A |
| 210 | SZ | CS | 43 | F | 17 | absent | absent | 3 | Quetiapine |
| | | | | | | | | | fumerate, |
| | | | | | | | | | Zuclopenthixol |
| 211 | CTRL | N/A | 28 | F | N/A | absent | absent | N/A | N/A |
| 212 | SZ | CS | 31 | M | 20 | present | present | 3 | Flupenthixol |
| | | | | | | | | | decanoate, |
| | | | | | | | | | Quetiapine |
| | | | | | | | | | fumerate |
| 213 | CTRL | N/A | 22 | M | N/A | absent | absent | N/A | N/A |
| 214 | SZ | CS | 46 | F | 43 | absent | present | 3 | Aripiprazole, |
| | | | | | | | | | Sertraline |
| 215 | CTRL | N/A | 55 | F | N/A | absent | present | N/A | N/A |
| 216 | SZ | CS | 42 | M | 27 | absent | present | 3 | Risperidone |
| 217 | SZ | CS | 21 | M | 16 | absent | present | 2 | Clozapine, |
| | | | | | | | | | Fluvoxamine |
| 218 | SZ | CD | 44 | M | 23 | absent | absent | 3 | Risperidone |
| 219 | SZ | CS | 36 | M | 33 | absent | present | 2 | Risperidone |
| 220 | SZ | CD | 37 | F | 35 | absent | present | 3 | Sertraline |
| 221 | SZ | CD | 37 | M | 22 | absent | present | 3 | Aripiprazole |
| 222 | SZ | CD | 30 | M | 24 | present | present | 2 | Ziprasidone |
| | | | | | | | | | hydrochloride, |
| | | | | | | | | | Moclobemide |
| 223 | SZ | CD | 33 | M | 26 | absent | absent | 3 | Olanzapine |
| 224 | SZ | CS | 26 | M | 19 | present | present | 2 | Risperidone |
| 225 | SZ | CS | 39 | M | 25 | absent | present | 3 | Olanzapine |
| 226 | CTRL | N/A | 32 | F | N/A | absent | present | N/A | N/A |
| 227 | SZ | CS | 44 | M | 24 | absent | present | 2 | Aripiprazole |
| 228 | SZ | CS | 27 | F | 26 | absent | present | 2 | Risperidone |
| 229 | SZ | CD | 49 | F | 18 | present | present | 3 | Clozapine |
| 230 | SZ | CS | 31 | M | 23 | absent | absent | 2 | Haloperidol |
| | | | | | | | | | decanoate, |
| | | | | | | | | | Quetiapine |
| | | | | | | | | | fumerate, |
| | | | | | | | | | Venlafaxine |
| 231 | SZ | CS | 29 | M | 28 | present | present | 2 | Aripiprazole, |
| | | | | | | | | | Venlafaxine |
| 232 | SZ | CD | 58 | M | 20 | absent | absent | 2 | N/A |
| 233 | SZ | CS | 34 | M | 19 | present | present | 2 | N/A |
| 234 | SZ | CD | 56 | M | 51 | absent | present | 3 | Clozapine |
| 235 | CTRL | N/A | 21 | M | N/A | absent | absent | N/A | N/A |
| 236 | SZ | CS | 42 | M | 23 | absent | absent | 3 | Zuclopenthixol |
| 237 | SZ | CD | 37 | M | 27 | absent | present | 2 | Risperidone |
| 238 | SZ | CS | 47 | M | 43 | absent | present | 3 | Mirtazapine, |
| | | | | | | | | | Venlafaxine |
| 239 | CTRL | N/A | 54 | F | N/A | absent | present | N/A | N/A |
| 240 | CTRL | N/A | 64 | M | N/A | absent | present | N/A | N/A |
| 241 | CTRL | N/A | 59 | F | N/A | absent | absent | N/A | N/A |
| 242 | SZ | CS | 57 | F | 25 | present | present | 3 | Trifluoperazine |
| | | | | | | | | | hydrochloride |
| 243 | CTRL | N/A | 47 | F | N/A | absent | absent | N/A | N/A |
| 244 | CTRL | N/A | 32 | M | N/A | absent | present | N/A | N/A |
| 245 | CTRL | N/A | 65 | F | N/A | absent | absent | N/A | N/A |
| 246 | SZ | CS | 35 | F | 27 | absent | present | 2 | Risperidone, |
| | | | | | | | | | Fluoxetine |
| 247 | CTRL | N/A | 56 | F | N/A | absent | absent | N/A | N/A |
| 248 | SZ | CS | 58 | F | 34 | present | present | 3 | Olanzapine, |
| | | | | | | | | | Venlafaxine |
| 249 | SZ | CS | 26 | M | 20 | present | present | 2 | Sertraline |
| 250 | SZ | CD | 48 | M | 32 | present | absent | 3 | Clozapine |
| 251 | SZ | CD | 39 | M | 28 | absent | present | 2 | Haloperidol |
| | | | | | | | | | decanoate, |
| | | | | | | | | | Quetiapine |
| | | | | | | | | | fumerate |
| 252 | CTRL | N/A | 55 | F | N/A | absent | present | N/A | N/A |
| 253 | SZ | CD | 39 | M | 23 | absent | present | 2 | Clozapine |
| 254 | CTRL | N/A | 55 | M | N/A | absent | absent | N/A | N/A |
| 255 | CTRL | N/A | 22 | F | N/A | absent | absent | N/A | N/A |
| 256 | CTRL | N/A | 38 | F | N/A | absent | present | N/A | N/A |
| 257 | CTRL | N/A | 30 | M | N/A | absent | present | N/A | N/A |
| 258 | SZ | CD | 46 | M | 20 | absent | present | 2 | Pericyazine |
| 259 | CTRL | N/A | 58 | M | N/A | absent | present | N/A | N/A |
| 260 | CTRL | N/A | 53 | M | N/A | absent | present | N/A | N/A |
| 261 | CTRL | N/A | 59 | M | N/A | absent | present | N/A | N/A |
| 262 | SZ | CS | 26 | M | 18 | absent | present | 3 | Olanzapine |
| 263 | CTRL | N/A | 50 | M | N/A | absent | absent | N/A | N/A |
| 264 | CTRL | N/A | 56 | F | N/A | absent | present | N/A | N/A |
| 265 | CTRL | N/A | 33 | F | N/A | absent | present | N/A | N/A |
| 266 | CTRL | N/A | 24 | M | N/A | absent | absent | N/A | N/A |
| 267 | CTRL | N/A | 47 | F | N/A | absent | absent | N/A | N/A |
| 268 | CTRL | N/A | 33 | M | N/A | absent | present | N/A | N/A |
| 269 | CTRL | N/A | 45 | M | N/A | absent | present | N/A | N/A |
| 270 | CTRL | N/A | 59 | F | N/A | absent | absent | N/A | N/A |
| 271 | CTRL | N/A | 47 | M | N/A | absent | absent | N/A | N/A |
| 272 | CTRL | N/A | 22 | M | N/A | absent | absent | N/A | N/A |
| 273 | CTRL | N/A | 24 | M | N/A | absent | present | N/A | N/A |
| 274 | CTRL | N/A | 56 | M | N/A | absent | absent | N/A | N/A |
| 275 | CTRL | N/A | 28 | F | N/A | absent | present | N/A | N/A |
| 276 | CTRL | N/A | 42 | M | N/A | absent | absent | N/A | N/A |
| 277 | CTRL | N/A | 31 | F | N/A | absent | present | N/A | N/A |
| 278 | CTRL | N/A | 56 | M | N/A | absent | present | N/A | N/A |
| 279 | SZ | CS | 51 | F | 40 | absent | present | 3 | Risperidone, |
| | | | | | | | | | Escitalopram |
| 280 | SZ | CS | 51 | F | 39 | absent | present | 3 | Olanzapine |
| 281 | CTRL | N/A | 29 | F | N/A | absent | present | N/A | N/A |
| 282 | CTRL | N/A | 36 | F | N/A | absent | present | N/A | N/A |
| 283 | CTRL | N/A | 57 | M | N/A | absent | present | N/A | N/A |
| 284 | CTRL | N/A | 61 | F | N/A | absent | present | N/A | N/A |
| 285 | CTRL | N/A | 53 | M | N/A | absent | present | N/A | N/A |
| 286 | CTRL | N/A | 59 | M | N/A | absent | present | N/A | N/A |
| 287 | CTRL | N/A | 51 | M | N/A | absent | present | N/A | N/A |
| 288 | CTRL | N/A | 50 | F | N/A | absent | present | N/A | N/A |
| 289 | CTRL | N/A | 22 | M | N/A | absent | absent | N/A | N/A |
| 290 | CTRL | N/A | 39 | F | N/A | absent | present | N/A | N/A |
| 291 | CTRL | N/A | 55 | F | N/A | absent | present | N/A | N/A |
| 292 | CTRL | N/A | 27 | F | N/A | absent | present | N/A | N/A |
| 293 | SZ | CD | 46 | M | 19 | present | present | 2 | Amisulpride, |
| | | | | | | | | | Clozapine |
| 294 | CTRL | N/A | 28 | M | N/A | absent | absent | N/A | N/A |
| 295 | CTRL | N/A | 18 | F | N/A | absent | present | N/A | N/A |
| 296 | CTRL | N/A | 22 | M | N/A | absent | absent | N/A | N/A |
| 297 | SZ | CD | 59 | M | 24 | absent | present | 2 | Amisulpride, |
| | | | | | | | | | Clozapine |
| 298 | CTRL | N/A | 48 | M | N/A | absent | present | N/A | N/A |
| 299 | CTRL | N/A | 42 | F | N/A | present | present | N/A | N/A |
| 300 | CTRL | N/A | 63 | F | N/A | absent | present | N/A | N/A |
| 301 | CTRL | N/A | 45 | M | N/A | absent | present | N/A | N/A |
| 302 | CTRL | N/A | 50 | F | N/A | absent | present | N/A | N/A |
| 303 | CTRL | N/A | 31 | M | N/A | absent | present | N/A | N/A |
| 304 | CTRL | N/A | 20 | F | N/A | absent | absent | N/A | N/A |
| 305 | SZ | CS | 21 | M | 18 | present | present | 2 | Quetiapine |
| | | | | | | | | | fumerate, |
| | | | | | | | | | Risperidone, |
| | | | | | | | | | Venlafaxine |
| 306 | CTRL | N/A | 55 | F | N/A | absent | present | N/A | N/A |
| 307 | CTRL | N/A | 39 | M | N/A | absent | present | N/A | N/A |
| 308 | CTRL | N/A | 49 | F | N/A | absent | present | N/A | N/A |
| 309 | CTRL | N/A | 63 | M | N/A | absent | present | N/A | N/A |
| 310 | CTRL | N/A | 56 | M | N/A | absent | absent | N/A | N/A |
| 311 | CTRL | N/A | 48 | M | N/A | absent | absent | N/A | N/A |
| 312 | CTRL | N/A | 39 | F | N/A | absent | absent | N/A | N/A |
| 313 | CTRL | N/A | 62 | F | N/A | absent | present | N/A | N/A |
| 314 | SZ | CD | 60 | M | 43 | absent | absent | 3 | Clozapine, |
| | | | | | | | | | Zuclopenthixol |
| 315 | SZ | CD | 25 | M | 21 | absent | present | 2 | Aripiprazole, |
| | | | | | | | | | Quetiapine |
| | | | | | | | | | fumerate |
| 316 | CTRL | N/A | 57 | F | N/A | absent | present | N/A | N/A |
| 317 | CTRL | N/A | 64 | F | N/A | absent | present | N/A | N/A |
| 318 | CTRL | N/A | 39 | F | N/A | present | present | N/A | N/A |
| 319 | CTRL | N/A | 37 | M | N/A | absent | absent | N/A | N/A |
| 320 | CTRL | N/A | 25 | F | N/A | absent | present | N/A | N/A |
| 321 | CTRL | N/A | 57 | M | N/A | absent | absent | N/A | N/A |
| 322 | CTRL | N/A | 58 | F | N/A | absent | present | N/A | N/A |
| 323 | CTRL | N/A | 45 | M | N/A | absent | present | N/A | N/A |
| 324 | SZ | CS | 28 | M | 16 | absent | absent | 2 | Aripiprazole |
| 325 | CTRL | N/A | 57 | F | N/A | absent | present | N/A | N/A |
| 326 | CTRL | N/A | 47 | M | N/A | absent | present | N/A | N/A |
| 327 | CTRL | N/A | 50 | M | N/A | absent | present | N/A | N/A |
| 328 | CTRL | N/A | 61 | M | N/A | absent | absent | N/A | N/A |
| 329 | SZ | CD | 37 | F | 28 | absent | present | 3 | Aripiprazole, |
| | | | | | | | | | Zuclopenthixol |
| 330 | CTRL | N/A | 43 | F | N/A | absent | present | N/A | N/A |
| 331 | CTRL | N/A | 54 | F | N/A | absent | present | N/A | N/A |
| 332 | SZ | CD | 43 | F | 33 | absent | present | 3 | Aripiprazole |
| 333 | CTRL | N/A | 51 | F | N/A | absent | present | N/A | N/A |
| 334 | CTRL | N/A | 34 | M | N/A | absent | absent | N/A | N/A |
| 335 | CTRL | N/A | 22 | M | N/A | absent | absent | N/A | N/A |
| 336 | CTRL | N/A | 43 | F | N/A | absent | present | N/A | N/A |
| 337 | CTRL | N/A | 47 | M | N/A | absent | present | N/A | N/A |
| 338 | CTRL | N/A | 28 | F | N/A | absent | absent | N/A | N/A |
| 339 | CTRL | N/A | 62 | F | N/A | absent | present | N/A | N/A |
| 340 | CTRL | N/A | 63 | M | N/A | absent | absent | N/A | N/A |
| 341 | CTRL | N/A | 62 | M | N/A | absent | present | N/A | N/A |
| 342 | CTRL | N/A | 64 | F | N/A | absent | present | N/A | N/A |
| 343 | CTRL | N/A | 58 | M | N/A | absent | present | N/A | N/A |
| 344 | CTRL | N/A | 47 | F | N/A | absent | present | N/A | N/A |
| 345 | SZ | CD | 42 | M | 30 | absent | absent | 3 | N/A |
| 346 | CTRL | N/A | 58 | M | N/A | absent | absent | N/A | N/A |
| 347 | CTRL | N/A | 64 | F | N/A | absent | present | N/A | N/A |
| 348 | CTRL | N/A | 45 | F | N/A | absent | present | N/A | N/A |
| 349 | CTRL | N/A | 26 | M | N/A | absent | present | N/A | N/A |
| 350 | CTRL | N/A | 59 | M | N/A | absent | present | N/A | N/A |
| 351 | CTRL | N/A | 42 | F | N/A | absent | present | N/A | N/A |
| 352 | CTRL | N/A | 35 | F | N/A | absent | absent | N/A | N/A |
| 353 | CTRL | N/A | 39 | F | N/A | present | present | N/A | N/A |
| 354 | CTRL | N/A | 51 | F | N/A | absent | present | N/A | N/A |
| 355 | CTRL | N/A | 63 | M | N/A | absent | absent | N/A | N/A |
| 356 | CTRL | N/A | 54 | F | N/A | absent | present | N/A | N/A |
| 357 | SZ | CD | 49 | M | 13 | present | present | 2 | Clozapine, |
| | | | | | | | | | Mirtazapine |
| 358 | SZ | CD | 35 | F | 25 | absent | absent | 3 | Quetiapine |
| | | | | | | | | | fumerate, |
| | | | | | | | | | Risperidone |
| 359 | CTRL | N/A | 55 | M | N/A | absent | present | N/A | N/A |
| 360 | CTRL | N/A | 31 | F | N/A | absent | absent | N/A | N/A |
| 361 | CTRL | N/A | 40 | M | N/A | absent | present | N/A | N/A |
| 362 | CTRL | N/A | 39 | M | N/A | absent | present | N/A | N/A |
| 363 | SZ | CD | 57 | M | 14 | absent | absent | 2 | Olanzapine |
| 364 | CTRL | N/A | 46 | F | N/A | absent | present | N/A | N/A |
| 365 | CTRL | N/A | 30 | F | N/A | absent | absent | N/A | N/A |
| 366 | CTRL | N/A | 46 | F | N/A | absent | absent | N/A | N/A |
| 367 | SZ | CD | 28 | F | 24 | absent | present | 3 | Quetiapine |
| | | | | | | | | | fumerate, |
| | | | | | | | | | Fluoxetine |
| 368 | CTRL | N/A | 47 | M | N/A | absent | present | N/A | N/A |
| 369 | SZ | CS | 45 | F | 34 | absent | present | 3 | Flupenthixol |
| | | | | | | | | | decanoate, |
| | | | | | | | | | Fluoxetine |
| 370 | CTRL | N/A | 41 | M | N/A | absent | absent | N/A | N/A |
| 371 | SZ | CD | 62 | F | 32 | absent | present | 3 | Trifluoperazine |
| | | | | | | | | | hydrochloride |
| 372 | CTRL | N/A | 51 | M | N/A | absent | absent | N/A | N/A |
| 373 | CTRL | N/A | 22 | F | N/A | absent | present | N/A | N/A |
| 374 | CTRL | N/A | 54 | M | N/A | absent | present | N/A | N/A |
| 375 | CTRL | N/A | 29 | F | N/A | absent | present | N/A | N/A |
| 376 | CTRL | N/A | 53 | M | N/A | absent | present | N/A | N/A |
| 377 | CTRL | N/A | 28 | F | N/A | absent | present | N/A | N/A |
| 378 | CTRL | N/A | 40 | F | N/A | absent | present | N/A | N/A |
| 379 | CTRL | N/A | 50 | M | N/A | absent | absent | N/A | N/A |
| 380 | CTRL | N/A | 32 | F | N/A | absent | present | N/A | N/A |
| 381 | CTRL | N/A | 36 | F | N/A | absent | absent | N/A | N/A |
| 382 | CTRL | N/A | 40 | F | N/A | absent | present | N/A | N/A |
| 383 | CTRL | N/A | 49 | F | N/A | absent | present | N/A | N/A |
| 384 | CTRL | N/A | 56 | M | N/A | absent | absent | N/A | N/A |
| 385 | CTRL | N/A | 62 | F | N/A | absent | absent | N/A | N/A |
| 386 | CTRL | N/A | 55 | F | N/A | absent | present | N/A | N/A |
| 387 | CTRL | N/A | 37 | M | N/A | absent | absent | N/A | N/A |
| 388 | CTRL | N/A | 46 | M | N/A | absent | present | N/A | N/A |
| 389 | SZ | CD | 41 | M | 22 | absent | absent | 3 | Clozapine |
| 390 | CTRL | N/A | 39 | F | N/A | absent | present | N/A | N/A |
| 391 | CTRL | N/A | 52 | F | N/A | absent | present | N/A | N/A |
| 392 | CTRL | N/A | 56 | F | N/A | absent | present | N/A | N/A |
| 393 | CTRL | N/A | 52 | F | N/A | absent | present | N/A | N/A |
| 394 | CTRL | N/A | 53 | M | N/A | absent | present | N/A | N/A |
| 395 | CTRL | N/A | 30 | F | N/A | absent | present | N/A | N/A |
| 396 | CTRL | N/A | 55 | F | N/A | absent | present | N/A | N/A |
| 397 | CTRL | N/A | 32 | F | N/A | absent | present | N/A | N/A |
| 398 | SZ | CS | 54 | M | 18 | absent | absent | 3 | Clozapine, |
| | | | | | | | | | Sertraline |
| 399 | SZ | CS | 64 | M | 20 | absent | present | 3 | Zuclopenthixol |
| 400 | CTRL | N/A | 34 | F | N/A | absent | present | N/A | N/A |
| 401 | CTRL | N/A | 46 | M | N/A | absent | absent | N/A | N/A |
| 402 | CTRL | N/A | 49 | M | N/A | absent | absent | N/A | N/A |
| 403 | SZ | CD | 25 | M | 19 | absent | present | 2 | Flupenthixol |
| | | | | | | | | | decanoate, |
| | | | | | | | | | Olanzapine, |
| | | | | | | | | | Quetiapine |
| | | | | | | | | | fumerate |
| 404 | SZ | CD | 39 | M | 33 | absent | absent | 3 | N/A |
| 405 | SZ | CD | 36 | M | 16 | absent | absent | 2 | Clozapine, |
| | | | | | | | | | Zuclopenthixol |
| 406 | CTRL | N/A | 59 | F | N/A | present | present | N/A | N/A |
| 407 | CTRL | N/A | 19 | F | N/A | absent | present | N/A | N/A |
| 408 | CTRL | N/A | 41 | F | N/A | absent | absent | N/A | N/A |
| 409 | CTRL | N/A | 52 | F | N/A | absent | present | N/A | N/A |
| 410 | SZ | CS | 30 | M | 19 | absent | present | 3 | N/A |
| 411 | SZ | CD | 45 | M | 25 | absent | absent | 2 | N/A |
| 412 | SZ | CD | 48 | M | 22 | present | absent | 3 | Olanzapine, |
| | | | | | | | | | Zuclopenthixol |
| 413 | CTRL | N/A | 54 | F | N/A | absent | present | N/A | N/A |
| 414 | CTRL | N/A | 59 | F | N/A | absent | present | N/A | N/A |
| 415 | CTRL | N/A | 57 | F | N/A | absent | present | N/A | N/A |
| 416 | CTRL | N/A | 55 | M | N/A | absent | present | N/A | N/A |
| 417 | SZ | CS | 52 | F | 33 | absent | present | 2 | Amisulpride, |
| | | | | | | | | | Citalopram |
| 418 | SZ | CD | 41 | M | 28 | absent | absent | 3 | Zuclopenthixol |
| 419 | CTRL | N/A | 53 | M | N/A | absent | present | N/A | N/A |
| 420 | CTRL | N/A | 63 | M | N/A | absent | present | N/A | N/A |
| 421 | CTRL | N/A | 63 | M | N/A | absent | present | N/A | N/A |
| 422 | CTRL | N/A | 49 | F | N/A | absent | present | N/A | N/A |
| 423 | CTRL | N/A | 57 | M | N/A | absent | present | N/A | N/A |
| 424 | CTRL | N/A | 52 | M | N/A | absent | absent | N/A | N/A |
| 425 | CTRL | N/A | 43 | F | N/A | absent | present | N/A | N/A |
| 426 | CTRL | N/A | 48 | F | N/A | absent | present | N/A | N/A |
| 427 | CTRL | N/A | 61 | F | N/A | absent | absent | N/A | N/A |
| 428 | CTRL | N/A | 47 | F | N/A | absent | present | N/A | N/A |
| 429 | CTRL | N/A | 59 | F | N/A | absent | present | N/A | N/A |
| 430 | CTRL | N/A | 25 | F | N/A | absent | present | N/A | N/A |
| 431 | CTRL | N/A | 62 | F | N/A | absent | absent | N/A | N/A |
| 432 | CTRL | N/A | 61 | F | N/A | absent | present | N/A | N/A |
| 433 | CTRL | N/A | 60 | M | N/A | absent | present | N/A | N/A |
| 434 | CTRL | N/A | 55 | M | N/A | absent | absent | N/A | N/A |
| 435 | SZ | CD | 33 | M | 19 | absent | absent | 2 | Trifluoperazine |
| | | | | | | | | | hydrochloride |
| 436 | CTRL | N/A | 43 | F | N/A | absent | present | N/A | N/A |
| 437 | CTRL | N/A | 62 | F | N/A | absent | present | N/A | N/A |
| 438 | CTRL | N/A | 61 | F | N/A | absent | present | N/A | N/A |
| 439 | CTRL | N/A | 27 | F | N/A | absent | present | N/A | N/A |
| 440 | CTRL | N/A | 54 | F | N/A | absent | present | N/A | N/A |
| 441 | CTRL | N/A | 54 | F | N/A | absent | present | N/A | N/A |
| 442 | CTRL | N/A | 39 | F | N/A | absent | present | N/A | N/A |
| 443 | SZ | CS | 40 | F | 36 | absent | present | 2 | Quetiapine |
| | | | | | | | | | fumerate |
| 444 | CTRL | N/A | 42 | F | N/A | present | present | N/A | N/A |
| 445 | CTRL | N/A | 54 | M | N/A | absent | present | N/A | N/A |
| 446 | CTRL | N/A | 46 | M | N/A | absent | present | N/A | N/A |
| 447 | CTRL | N/A | 60 | M | N/A | absent | present | N/A | N/A |
| 448 | CTRL | N/A | 49 | F | N/A | absent | present | N/A | N/A |
| 449 | CTRL | N/A | 56 | F | N/A | absent | present | N/A | N/A |
| 450 | CTRL | N/A | 57 | M | N/A | absent | present | N/A | N/A |
| 451 | CTRL | N/A | 18 | M | N/A | absent | absent | N/A | N/A |
| 452 | CTRL | N/A | 61 | M | N/A | absent | present | N/A | N/A |
| 453 | SZ | CD | 33 | M | 20 | present | present | 2 | Clozapine |
| 454 | CTRL | N/A | 46 | F | N/A | absent | present | N/A | N/A |
| 455 | SZ | CS | 38 | M | 22 | absent | absent | 2 | Olanzapine |
| 456 | SZ | CS | 24 | F | 14 | absent | present | 3 | Quetiapine |
| | | | | | | | | | fumerate |
| 457 | SZ | CD | 40 | M | 21 | absent | absent | 3 | Aripiprazole, |
| | | | | | | | | | Clozapine, |
| | | | | | | | | | Fluoxetine |
| 458 | CTRL | N/A | 47 | M | N/A | absent | present | N/A | N/A |
| 459 | CTRL | N/A | 64 | M | N/A | absent | present | N/A | N/A |
| 460 | SZ | CD | 63 | M | 19 | present | present | 2 | Clozapine |
| 461 | CTRL | N/A | 19 | M | N/A | absent | present | N/A | N/A |
| 462 | SZ | CD | 38 | F | 18 | absent | absent | 2 | Flupenthixol |
| | | | | | | | | | decanoate, |
| | | | | | | | | | Quetiapine |
| | | | | | | | | | fumerate, |
| | | | | | | | | | Risperidone, |
| | | | | | | | | | Citalopram |
| 463 | CTRL | N/A | 59 | F | N/A | absent | absent | N/A | N/A |
| 464 | CTRL | N/A | 33 | F | N/A | absent | present | N/A | N/A |
| 465 | SZ | CD | 36 | M | 19 | absent | present | 3 | Clozapine |
| 466 | SZ | CD | 37 | M | 25 | absent | present | 2 | Clozapine |
| 467 | CTRL | N/A | 28 | F | N/A | absent | present | N/A | N/A |
| 468 | CTRL | N/A | 60 | F | N/A | absent | present | N/A | N/A |
| 469 | CTRL | N/A | 48 | M | N/A | absent | present | N/A | N/A |
| 470 | SZ | CD | 41 | F | 20 | present | present | 2 | Clozapine |
| 471 | CTRL | N/A | 56 | M | N/A | absent | absent | N/A | N/A |
| 472 | SZ | CD | 40 | M | 21 | absent | absent | 2 | Aripiprazole, |
| | | | | | | | | | Clozapine, |
| | | | | | | | | | Escitalopram |
| 473 | CTRL | N/A | 61 | F | N/A | absent | present | N/A | N/A |
| 474 | CTRL | N/A | 42 | F | N/A | absent | present | N/A | N/A |
| 475 | CTRL | N/A | 46 | F | N/A | absent | present | N/A | N/A |
| 476 | CTRL | N/A | 65 | F | N/A | absent | present | N/A | N/A |
| 477 | CTRL | N/A | 45 | M | N/A | absent | present | N/A | N/A |

SZ—Schizophrenia; CTRL—Control; CD—Cognitive deficit subtype; CS—Cognitive spared subtype; F—Female; M—Male

### Immuno-Fractionation and RNA Extraction

Protein G magnetic beads (Dynabeads protein G for IP; catalogue #10004D) were washed three times in PBS, 50 μL beads incubated with 4 μL anti-MAP1B (rabbit, polyclonal, anti-MAP1B, catalogue #A301-446A, supplied by Bethyl laboratories) for one hour at room temperature and then washed again to remove unbound antibody. 100 μL serum plus protease inhibitor was incubated with pre-coupled beads overnight at 4° C. with end on-end rotation. Following overnight incubation, serum tubes were placed on the magnetic rack to allow fractionation. The supernatant (serum depleted of EV's expressing MAP1B) was removed and immediately transferred to clean tubes and placed in 80° C. storage. Meanwhile, the beads were washed 3 times in PBS and incubated in 100 mM glycine buffer (pH 3.0) for three minutes to release bound EV's into the supernatant. The supernatant was collected in a clean tube for immediate RNA extraction. Total RNA was extracted from serum EV's using Trizol, according to the manufacturer's instructions. Briefly, 1 mL Trizol was added to 100 μL EV supernatant. Samples were homogenised using 23-gauge needle and rested at room temperature for 5 minutes. Chloroform (200 μL) was added, samples vortexed, rested and phases separated by centrifugation at 20,000×g for 15 minutes at 4° C. The upper aqueous phase containing RNA was transferred to a clean tube. Isopropyl alcohol (500 μL) and RNA grade glycogen (2 μL) were added, the sample vortexed and incubated overnight at −20° C. to precipitate total RNA. The following day, samples were centrifuged at 12,000×g for 20 minutes at 4° C., and the supernatant discarded. The RNA pellet was washed twice with 1 mL ice cold 75% ethanol, air dried, resuspended in 12 μL RNAse free water and stored at −80° C.

### Library Preparation and Next-Generation Sequencing

Small RNA libraries were prepared using SMARTer smRNA-seq kit for illumina (Clontech Laboratories Inc.) according to the manufacturer's instructions. Total RNA (7 μL) was polyadenylated, reverse transcribed and amplified (17 cycles). Post-PCR clean-up was performed according to the manufacturer's instructions. Libraries were quantified (KAPA Biosystems and Agilent smallRNA chip), equimolar-pooled and size selected (8% acrylamide, native PAGE) by excising fragments corresponding to approximately 175 base pairs. Sequencing was performed on NovaSeq 6000 using XP workflow (2-lane flow cell) for 100 cycles of paired-end reads.

### Differential Expression Analysis

Raw sequencing reads were demultiplexed and quality checked using FastQC. Reads were trimmed of adapters, low quality bases and length <10 using Cutadapt (Martin, 2011). Sequencing reads were aligned using Bowtie2 (Langmead and Salzberg, 2012) to the human genome (GRCh38) and features counted using HTseq (Anders, Pyl and Huber, 2015) according to miRbase annotation of mature miRNA (v22.1). Differential expression of miRNA was analysed using edgeR (McCarthy, Chen and Smyth, 2012). Read counts were filtered to retain miRNA that reached 10 counts per million (CPM) in the smallest group size (n=221) and were normalised using trimmed mean of M-values.

Differential expression was determined using the likelihood ratio test, where a p-value<0.05 and False Discovery Rate <0.1 were the criteria for significant differential expression.

The set of miRNA robustly detected in the whole cohort was tested for overrepresentation using TAM2.0 (J. Li et al., 2018) with background being all miRNA in the curated database. Heat map was generated using the R package ComplexHeatmap (Gu, Eils and Schlesner, 2016) on normalised and scaled counts per million (CPM) and the distance matrix ordered using the seriation package (Hahsler, Hornik and Buchta, 2008) by minimising the sum of dissimilarities. Principal component analysis was performed using FactoMineR (Lê, Josse and Husson, 2008) on normalised and scaled CPM.

miRNA Target Genes and Functional Analysis

Prediction of miRNA targets was performed with TargetScan (Agarwal et al., 2015). Human transcripts with 3′UTR sequences complementary to the seed sequence of at least two miRNA (schizophrenia versus control, cognitive deficit versus cognitively spared) or three miRNA (cognitive deficit versus control, TRS versus non-TRS) were extracted. The stringency was optimized by filtering the predicted targets so that gene lists were of manageable and similar size. Gene lists were analysed using the ToppFun suite with the default background gene set and the top five significantly enriched categories were selected, sorted by q-value (Bonferroni) (Chen et al., 2009). To identify pathways enriched with cognitive deficit associated miRNA targets, the gene list was submitted to Consensus Path Database and analysed with default settings (Kamburov et al., 2011). Genes from selected pathways were tested for enrichment of protein drug targets using WebGestalt and results are presented for significant enrichment using multiple testing adjusted P value<0.05 (Liao et al, 2019). Additionally, pathway genes were clustered by protein-protein interactions with the following settings; minimum interaction score 0.70 (high confidence) and inflation parameter 4 in the Markov cluster algorithm, as implemented in STRING (Szklarczyk et al., 2019).

Gene-Set Association of miRNA Target Genes

Schizophrenia GWAS summary statistics were obtained from the schizophrenia working group of the psychiatric genomics consortium (PGC) for 161405 participants, of which the majority are of European ancestry (Schizophrenia Working Group of the Psychiatric Genomics Consortium et al., 2020).

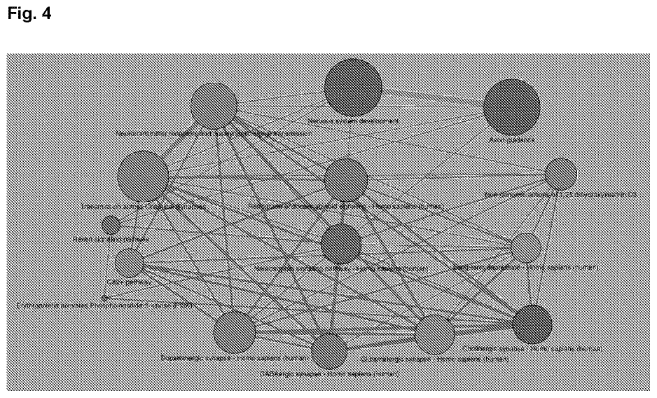

Gene-set association of the predicted target genes of differentially expressed miRNA was tested using MAGMA (Leeuw et al., 2015), as described elsewhere (Reay et al., 2018). Briefly, MAGMA aggregates SNP-wise P values at gene-level to act as the unit of effect in a test of gene-set association. Gene-based P values were calculated using the snp-wise=mean MAGMA model, whereby the linkage disequilibrium (LD) estimated between SNPs is leveraged to approximate the null χ\hat{ }2 distribution. The 1000 genomes phase 3 European reference panel was used to provide the LD estimations. Autosomal gene coordinates in hg19 assembly were obtained from NCBI and the 1000 genomes phase 3 European panel utilised as an LD reference. Genes within the MHC region were not considered due to the haplotype complexity of that region, as is usual practice (Wray et al, 2018; Reay et al, 2020). We extended the boundaries of the genic coordinates upstream and downstream to capture regulatory variation with both a conservative (5 kilobases (kb) upstream, 1.5 kb downstream) and liberal genic (35 kb upstream, 10 kb downstream) boundary definition used for analyses. After probit transformation of gene-based P to Z, MAGMA constructs a linear regression model such that Z was the outcome and a binary indicator of gene-set membership an explanatory variable to test whether genes in the set were more associated than all other genes considered. This model was covaried for confounders including gene-size and minor allele count (Leeuw et al., 2015; Leeuw et al., 2016). Given that the gene-sets considered are highly brain expressed, we additionally constructed a model covaried for the cortical expression of each gene (median transcript per million, PsychENCODE dorsolateral prefrontal cortex dataset) to assess whether this may bias the estimated association (see FIG. 4a) (Reay & Cairns, 2020; Leeuw et al., 2018; Gandal et al., 2018). Furthermore, quantile-quantile (QQ) plots were generated to visualise expected and observed residual genic Z scores from a null model without the gene-set of interest, as outlined elsewhere (see FIG. 4b) (Leeuw et al., 2018). The plot should start to deviate from the diagonal early and consistently if the association is not driven by a subset of genes in the set. An upper, one-sided 95% confidence band was plotted which represents deviation from the plot diagonal by chance.

### Results

The Serum miRNA Profile Featured miR-17~92 Cluster Molecules

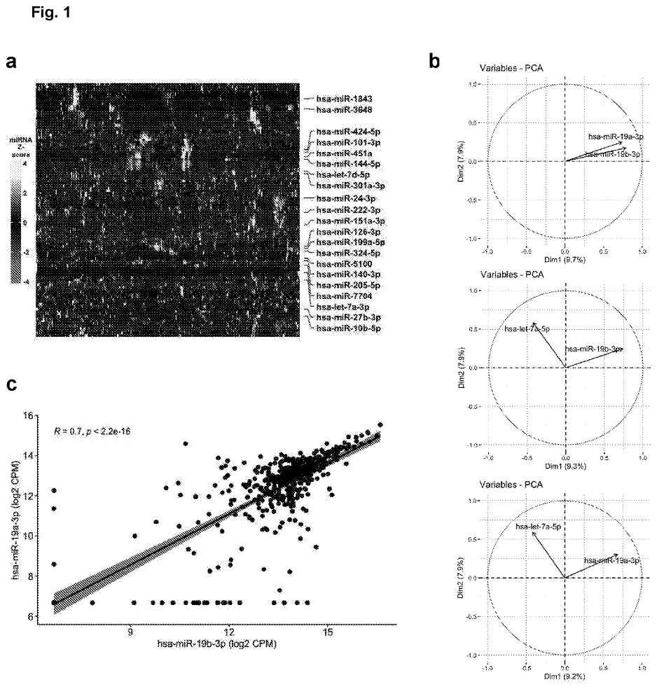

Initial analysis sought to identify the overall miRNA profile of putative brain associated molecules from neuronal enrichment of serum EV's, irrespective of diagnostic category (FIG. 1a). Enrichment analysis for miRNA with robust expression across all samples revealed tissue specificity for the brain (cerebellum, p=1.08e-16, Bonferroni=6.56e-14), the miR-17~92 cluster (p=3.71e-06, Bonferroni=2.25e-03), the let-7 family (p=9.46e-16, Bonferroni=5.74e-13) and miR-29 family (p=1.14e-05, Bonferroni=6.93e-03) (Table 4). Furthermore, principal components analysis identified miR-19a and miR-19b (members of miR-17~92 cluster) as explaining the largest proportion of variation in the data (FIG. 1b). Given that miR-19a and miR-19b can arise from the same primary transcript and show evidence of a positive monotonic association (FIG. 1c) (but no association for the other cluster molecules, Table 5), the present inventors repeated principal components analysis with each molecule removed and found each alone best describes the data (FIG. 1b).

TABLE 4
Enrichment analysis of putative brain associated miRNA expressed in the full
cohort.

| Category | Term | Count | P | Bonferroni | FDR | miRNA |
|---|---|---|---|---|---|---|
| Tissue | Brain | 16 | 1.08e−16 | 6.56e−14 | 6.22e−15 | miR-16-1, miR-16-2, miR- |
| specificity | (cerebellum) | | | | | 185, miR-19b-1, miR-19b-2, |
| | | | | | | miR-93, miR-451a, miR-25, |
| | | | | | | miR-223, miR-144, miR- |
| | | | | | | 15b, miR-19a, miR-486-1, |
| | | | | | | miR-486-2, miR-20a, miR- |
| | | | | | | 17 |
| Cluster | miR-17 | 5 | 3.71e−06 | 2.25e−03 | 4.56e−05 | miR-17, miR-19a, miR-20a, |
| | cluster | | | | | miR-19b-1, miR-92a-1 |
| Family | let-7 family | 12 | 9.46e−16 | 5.74e−13 | 4.96e−14 | let-7a-1, let-7a-2, let-7a-3, |
| | | | | | | let-7b, let-7c, let-7d, let-7e, |
| | | | | | | let-7f-1, let-7f-2, let-7g, let- |
| | | | | | | 7i, miR-98 |
| Family | miR-29 | 4 | 1.14e−05 | 6.93e−03 | 1.27e−04 | miR-29a, miR-29b-1, miR- |
| | family | | | | | 29b-2, miR-29c |

TABLE 5
Correlation values for miR-17~92 cluster molecules.

| | miR-17-5p | miR-19a-3p | miR-19b-3p | miR-20a-5p | miR-92a-3p | miR-25-3p | miR-93-5p |
|---|---|---|---|---|---|---|---|
| miR-17-5p | 1.00 | 0.36 | 0.38 | 0.36 | 0.28 | 0.05 | 0.42 |
| miR-19a-3p | 0.36 | 1.00 | 0.70 | 0.40 | 0.17 | 0.17 | 0.34 |
| miR-19b-3p | 0.38 | 0.70 | 1.00 | 0.41 | 0.14 | 0.15 | 0.33 |
| miR-20a-5p | 0.36 | 0.40 | 0.41 | 1.00 | 0.40 | 0.08 | 0.35 |
| miR-92a-3p | 0.28 | 0.17 | 0.14 | 0.40 | 1.00 | 0.17 | 0.38 |
| miR-25-3p | 0.05 | 0.17 | 0.15 | 0.08 | 0.17 | 1.00 | 0.25 |
| miR-93-5p | 0.42 | 0.34 | 0.33 | 0.35 | 0.38 | 0.25 | 1.00 |

Spearman's correlation analysis indicates expression of hsa-miR-19a-3p and hsa-miR-19b-3p have a positive monotonic association (Spearman's rho 0.70).

Differential Expression of miRNA for Schizophrenia Subjects and Non-Psychiatric Controls

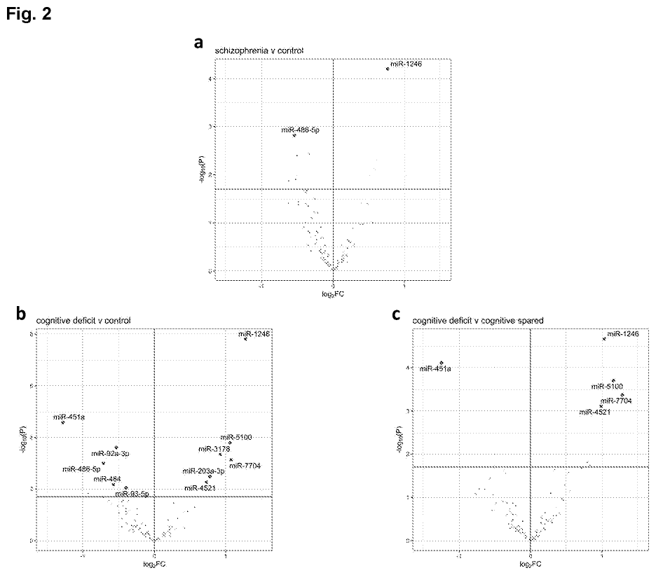

The expression of brain derived miRNA in serum collected from study participants with schizophrenia (n=221) and non-psychiatric controls (n=256) was investigated by sequencing small RNA of immuno-fractionated extracellular vesicles enriched for neuronal origin by virtue of the neuron specific membrane antigen, MAP1B. Prior to analysis, libraries with overall low read counts and miRNA with low expression were removed, followed by library normalisation to account for variations in sequencing depth. A generalized linear model was used to estimate log CPM (counts per million) for each miRNA while adjusting for covariates (batch, gender and age) and the difference in expression between groups determined using the likelihood ratio test (LRT) as implemented in edgeR. This analysis revealed two miRNA significantly altered (FDR<0.1) between groups (Table 6). Specifically, miR-486-5p was observed to display reduced expression, while miR-1246 demonstrated increased expression in schizophrenia subjects (FIG. 2a).

TABLE 6
miRNA differentially expressed in schizophrenia subjects.

| | | |
|---|---|---|
| | log₂ fold change (95% | |
| mature miRNA name | confidence interval) | false discovery rate |

Schizophrenia compared to controls

| | | |
|---|---|---|
| hsa-miR-1246 | 0.764 [0.39, 1.14] | 6.51E−03 |
| hsa-miR-486-5p | −0.540 [−0.87, −0.21] | 7.98E−02 |

Differential Expression of miRNA in Cognitive Subtypes of Schizophrenia Cases and Controls

The data was analysed to test for differential expression of miRNA between cognitive subtypes in schizophrenia subjects and non-psychiatric controls, using the same pipeline and subjects as above. This analysis revealed striking differences in schizophrenia subjects with cognitive deficit compared to both non-psychiatric controls and cognitively spared schizophrenia subjects. Eleven miRNA were differentially expressed in cognitive deficit compared to control subjects (FDR<0.1), with six miRNA showing increased and five miRNA showing decreased expression (FIG. 2b). Additionally, four of the eleven miRNA showed an absolute log 2 fold-change greater than 1.0 (Table 7). When the present inventors directly compared the cognitive deficit to cognitive spared subtype of schizophrenia, four miRNA were observed to display increased expression while another was observed to have decreased expression (FIG. 2c). Three of the five miRNA were observed to have an absolute log 2 fold-change greater than 1.0 (Table 7). By contrast when the present inventors compared the cognitive spared subtype with control subjects they did not observe any differentially expressed miRNA, although seven molecules with reduced expression were nominally significant (p-value<0.05), (Table 7).

TABLE 7
miRNA differentially expressed in cognitive
subtypes of schizophrenia subjects.

| | | |
|---|---|---|
| | log₂ fold change (95% | |
| mature miRNA name | confidence interval) | false discovery rate |

Cognitive deficit compared to controls

| | | |
|---|---|---|
| hsa-miR-1246 | 1.278 [0.83, 1.72] | 1.60E−06 |
| (SEQ ID NO: 1) | | |
| hsa-miR-451a | −1.278 [−1.87, −0.68] | 1.38E−03 |
| (SEQ ID NO: 8) | | |
| hsa-miR-5100 | 1.061 [0.51, 1.61] | 5.62E−03 |
| (SEQ ID NO: 3) | | |
| hsa-miR-92a-3p | −0.533 [−0.82, −0.25] | 6.62E−03 |
| (SEQ ID NO: 9) | | |
| hsa-miR-3178 | 0.926 [0.41, 1.45] | 9.80E−03 |
| (SEQ ID NO: 4) | | |
| hsa-miR-7704 | 1.076 [0.45, 1.70] | 1.31E−02 |
| (SEQ ID NO: 5) | | |
| hsa-miR-486-5p | −0.713 [−1.14, −0.29] | 1.52E−02 |
| (SEQ ID NO: 2) | | |
| hsa-miR-203a-3p | 0.778 [0.26, 1.30] | 4.34E−02 |
| (SEQ ID NO: 6) | | |
| hsa-miR-4521 | 0.730 [0.22, 1.24] | 6.37E−02 |
| (SEQ ID NO: 7) | | |
| hsa-miR-484 | −0.572 [−0.99, −0.16] | 7.10E−02 |
| (SEQ ID NO: 10) | | |
| hsa-miR-93-5p | −0.396 [−0.69, −0.10] | 8.64E−02 |
| (SEQ ID NO: 11) | | |

Cognitively spared compared to controls

| | | |
|---|---|---|
| hsa-miR-12136 | −0.620 [−1.03, −0.21] | 0.35 |
| (SEQ ID NO: 12) | | |
| hsa-miR-324-5p | −0.755 [−1.32, −0.19] | 0.49 |
| (SEQ ID NO: 13) | | |
| hsa-miR-26a-5p | −0.620 [−1.16, −0.08] | 0.54 |
| (SEQ ID NO: 14) | | |
| hsa-miR-146b-5p | −0.536 [−1.03, −0.04] | 0.54 |
| (SEQ ID NO: 15) | | |
| hsa-let-7d-3p | −0.441 [−0.85, −0.03] | 0.54 |
| (SEQ ID NO: 16) | | |
| hsa-miR-486-5p | −0.420 [−0.81, −0.03] | 0.54 |
| (SEQ ID NO: 2) | | |
| hsa-miR-425-5p | −0.441 [−0.88, −0.00] | 0.54 |
| (SEQ ID NO: 17) | | |

Cognitive deficit compared to cognitively spared

| | | |
|---|---|---|
| hsa-miR-1246 | 1.032 [0.56, 1.51] | 2.28E−03 |
| (SEQ ID NO: 1) | | |
| hsa-miR-451a | −1.247 [−1.86, −0.63] | 4.05E−03 |
| (SEQ ID NO: 8) | | |
| hsa-miR-5100 | 1.158 [0.55, 1.77] | 6.93E−03 |
| (SEQ ID NO: 3) | | |
| hsa-miR-7704 | 1.284 [0.57, 2.00] | 1.11E−02 |
| (SEQ ID NO: 5) | | |
| hsa-miR-4521 | 0.988 [0.41, 1.56] | 1.63E−02 |
| (SEQ ID NO: 7) | | |

miRNA Differentially Expressed in Schizophrenia and Cognitive Subtype Subjects Target Synaptic Genes and Neuronal Biological Processes

To gain insight into the functional significance of miRNA differentially expressed in schizophrenia and cognitive subtype subjects, the present inventors analysed the predicted target genes and found enrichment for annotations to synaptic genes and neuronal biological processes. Specifically, in the comparison of schizophrenia subjects with cognitive deficit and non-psychiatric controls, the biological process analysis displayed neurogenesis as significantly enriched with the predicted target genes. Cellular component analysis revealed enrichment for these genes in synapse, post-synapse, axon and dendritic tree (Table 8). In the comparison of schizophrenia subjects with cognitive deficit compared to cognitively spared counterparts, the biological process analysis displayed generation of neurons, neurogenesis and neuron differentiation as significantly enriched with the predicted target genes. Cellular component analysis displayed target gene enrichment in synapse and axon (Table 9). Finally, in the comparison of schizophrenia subjects and non-psychiatric controls, the biological process analysis displayed regulation of synapse organisation and regulation of synapse structure or activity as significantly enriched with the predicted target genes. Cellular component analysis displayed target gene enrichment in integral component of postsynaptic specialisation membrane, integral component of postsynaptic membrane, postsynaptic density, asymmetric synapse and intrinsic component of postsynaptic specialisation membrane (Table 10).

TABLE 8
Gene and disease ontologies enriched for genes targeted by miRNA that are dysregulated
in schizophrenia subjects with cognitive deficit compared to controls.

| | | | | | |
|---|---|---|---|---|---|
| Ontology ID | Ontology name | n | % | p-value | Bonferroni |

GO: Biological process

| | | | | | |
|---|---|---|---|---|---|
| GO: 0051254 | positive regulation of RNA | 400 | 12.7 | 2.204E−15 | 2.368E−11 |
| | metabolic process | | | | |
| GO: 0022008 | neurogenesis | 413 | 13.1 | 1.607E−14 | 1.727E−10 |
| GO: 1903508 | positive regulation of nucleic | 378 | 12.0 | 1.690E−14 | 1.816E−10 |
| | acid-templated transcription | | | | |
| GO: 0045893 | positive regulation of | 378 | 12.0 | 1.690E−14 | 1.816E−10 |
| | transcription, DNA- | | | | |
| | templated | | | | |
| GO: 1902680 | positive regulation of RNA | 378 | 12.0 | 1.846E−14 | 1.984E−10 |
| | biosynthetic process | | | | |

GO: Cellular component

| | | | | | |
|---|---|---|---|---|---|
| GO: 0045202 | synapse | 357 | 11.3 | 5.970E−16 | 7.779E−13 |
| GO: 0030424 | axon | 197 | 6.2 | 2.558E−10 | 3.333E−7 |
| GO: 0098794 | postsynapse | 186 | 5.9 | 4.525E−10 | 5.896E−7 |
| GO: 0098791 | Golgi apparatus | 211 | 6.7 | 8.500E−10 | 1.108E−6 |
| | subcompartment | | | | |
| GO: 0097447 | dendritic tree | 191 | 6.1 | 8.877E−10 | 1.157E−6 |

GO: Molecular function

| | | | | | |
|---|---|---|---|---|---|
| GO: 0140110 | transcription regulator | 444 | 14.1 | 2.094E−16 | 5.172E−13 |
| | activity | | | | |
| GO: 0043565 | sequence-specific DNA | 403 | 12.8 | 7.969E−15 | 1.968E−11 |
| | binding | | | | |
| GO: 0001067 | transcription regulatory | 378 | 12.0 | 1.278E−14 | 3.158E−11 |
| | region nucleic acid binding | | | | |
| GO: 1990837 | sequence-specific double- | 384 | 12.2 | 2.886E−14 | 7.128E−11 |
| | stranded DNA binding | | | | |
| GO: 0000976 | transcription cis-regulatory | 375 | 11.9 | 3.560E−14 | 8.793E−11 |
| | region binding | | | | |

n = number of annotated genes from input list, % = n/total number of genes from input list (total 3156).

TABLE 9
Gene and disease ontologies enriched for genes targeted by miRNA
that are dysregulated in schizophrenia subjects with cognitive
deficit compared to cognitively spared schizophrenia subjects.

| | | | | | |
|---|---|---|---|---|---|
| Ontology ID | Ontology name | n | % | p-value | Bonferroni |

GO: Biological process

| | | | | | |
|---|---|---|---|---|---|
| GO: 0048699 | generation of neurons | 371 | 12.5 | 8.007E−15 | 8.467E−11 |
| GO: 0022008 | neurogenesis | 388 | 13.1 | 1.580E−13 | 1.671E−9 |
| GO: 0030182 | neuron differentiation | 335 | 11.3 | 5.378E−13 | 5.687E−9 |
| GO: 0000902 | cell morphogenesis | 254 | 8.6 | 9.027E−12 | 9.545E−8 |
| GO: 0048858 | cell projection | 181 | 6.1 | 1.765E−11 | 1.866E−7 |
| | morphogenesis | | | | |

GO: Cellular component

| | | | | | |
|---|---|---|---|---|---|
| GO: 0045202 | synapse | 322 | 10.9 | 3.203E−12 | 4.109E−9 |
| GO: 0098791 | Golgi apparatus | 203 | 6.9 | 1.822E−10 | 2.337E−7 |
| | subcompartment | | | | |
| GO: 0030424 | axon | 187 | 6.3 | 2.632E−10 | 3.377E−7 |
| GO: 0005794 | Golgi apparatus | 332 | 11.2 | 5.207E−10 | 6.681E−7 |
| GO: 0000139 | Golgi membrane | 174 | 5.9 | 1.850E−9 | 2.373E−6 |

GO: Molecular function

| | | | | | |
|---|---|---|---|---|---|
| GO: 0140110 | transcription regulator | 410 | 13.9 | 6.737E−14 | 1.623E−10 |
| | activity | | | | |
| GO: 0001067 | transcription regulatory | 353 | 11.9 | 2.932E−13 | 7.062E−10 |
| | region nucleic acid binding | | | | |
| GO: 0003700 | DNA-binding transcription | 318 | 10.8 | 1.285E−12 | 3.096E−9 |
| | factor activity | | | | |
| GO: 0000976 | transcription cis-regulatory | 349 | 11.8 | 1.311E−12 | 3.159E−9 |
| | region binding | | | | |
| GO: 1990837 | sequence-specific double- | 357 | 12.1 | 1.318E−12 | 3.174E−9 |
| | stranded DNA binding | | | | |

n = number of annotated genes from input list, % = n/total number of genes from input list (total 2958).

TABLE 10
Gene and disease ontologies enriched for genes targeted by miRNA that
are dysregulated in schizophrenia subjects compared to controls.

| | | | | | |
|---|---|---|---|---|---|
| Ontology ID | Ontology name | n | % | p-value | Bonferroni |

GO: Biological process

| | | | | | |
|---|---|---|---|---|---|
| GO: 0050807 | regulation of synapse | 26 | 3.6 | 1.690E−6 | 1.071E−2 |
| | organisation | | | | |
| GO: 0050803 | regulation of synapse structure | 26 | 3.6 | 4.086E−6 | 2.590E−2 |
| | or activity | | | | |

GO: Cellular component

| | | | | | |
|---|---|---|---|---|---|
| GO: 0099060 | integral component of | 14 | 2.0 | 1.563E−5 | 1.230E−2 |
| | postsynaptic specialization | | | | |
| | membrane | | | | |
| GO: 0099055 | integral component of | 18 | 2.5 | 2.123E−5 | 1.671E−2 |
| | postsynaptic membrane | | | | |
| GO: 0014069 | postsynaptic density | 33 | 4.6 | 2.304E−5 | 1.813E−2 |
| GO: 0032279 | asymmetric synapse | 33 | 4.6 | 2.304E−5 | 1.813E−2 |
| GO: 0098948 | intrinsic component of | 14 | 2.0 | 2.941E−5 | 2.314E−2 |
| | postsynaptic specialization | | | | |
| | membrane | | | | |

GO: Molecular function

| | | | | | |
|---|---|---|---|---|---|
| GO: 1990837 | sequence-specific double- | 110 | 15.4 | 7.341E−10 | 9.286E−7 |
| | stranded DNA binding | | | | |
| GO: 0003690 | double-stranded DNA binding | 114 | 15.9 | 9.334E−10 | 1.181E−6 |
| GO: 0000976 | transcription cis-regulatory | 107 | 15.0 | 1.339E−9 | 1.694E−6 |
| | region binding | | | | |
| GO: 0001067 | transcription regulatory region | 107 | 15.0 | 1.582E−9 | 2.002E−6 |
| | nucleic acid binding | | | | |
| GO: 0003700 | DNA-binding transcription | 98 | 13.7 | 2.106E−9 | 2.664E−6 |
| | factor activity | | | | |

n = number of annotated genes from input list, % = n/total number of genes from input list (total 715).

Cognitive Deficit-Associated miRNA Targets are Enriched for Schizophrenia Association

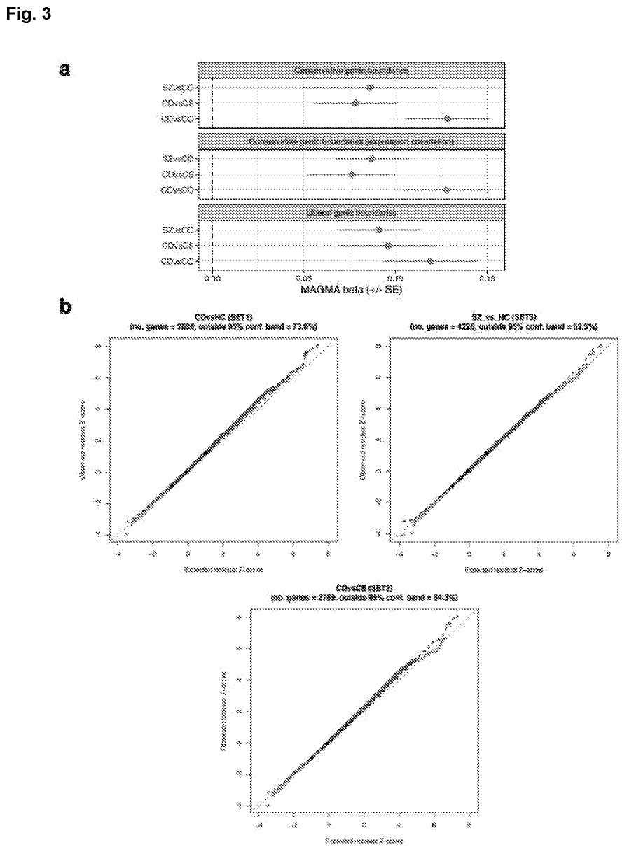

To further investigate the functional implications of the differentially expressed miRNA in relation to the molecular basis of disease, the present inventors tested whether their predicted targets were enriched with genes genetically associated with schizophrenia, as indexed by genetic variants from GWAS of the disorder. They observed that each of the three miRNA-associated gene sets displayed some evidence of association with schizophrenia, at both conservative and liberal genic boundaries and when adjusted for cortical expression of each gene (Table 11, FIG. 3a). Of the three gene sets tested using conservative genic boundaries, the schizophrenia subjects with cognitive deficit compared to non-psychiatric controls group was the most significantly enriched with common variant associations (β=0.089, SE=0.022, P=2.45×10-5), followed by the schizophrenia subjects with cognitive deficit compared to cognitively spared schizophrenia subjects (β=0.075, SE=0.022, P=4.30×10-4) and schizophrenia subjects compared to non-psychiatric controls group, where the association was more nominal (β=0.117, SE=0.043, P=3.66×10-3).

Among the most significant results within the three gene sets were transcription factor 4 (TCF4, P=3.45×10-17) and calcium voltage-gated channel subunit alpha1 C (CACNA1C, P=4.23×10-16).

TABLE 11
Gene-set association for predicted targets
of differentially expressed miRNA

| Cell-type gene-set | P_{Conservative}¹ | P_{Liberal}² |
|---|---|---|
| Schizophrenia vs non-psychiatric control | 3.66 × 10⁻³ | 0.03 |
| Cognitive deficit vs Cognitively spared | 4.30 × 10⁻⁴ | 1.49 × 10⁻⁴ |
| Cognitive deficit vs non-psychiatric controls | 2.45 × 10⁻⁵ | 7.25 × 10⁻⁴ |

¹Conservative refers to the genic boundaries for variant to gene annotation in MAGMA, that is, gene coordinates were extended 5 kb upstream and 1.5 kb downstream to capture regulatory variation,
²Liberal genic boundaries were 35 kb upstream and 10 kb downstream.

Cognitive Deficit Associated miRNA Regulate Genes Enriched in Neurotransmitter and Neurotrophin Signaling Pathways

Given the magnitude of miRNA alterations observed in schizophrenia subjects with cognitive deficit and the important role miRNA play in brain development and function, the present inventors investigated pathways predicted to be perturbed as a consequence of cognitive deficit associated miRNA. In accordance with the gene ontology analysis for the combined schizophrenia cases, several neuronal system pathways were enriched. For example, cholinergic synapse (q-value 0.001), neurotrophin signaling pathway (q-value 0.003) and reelin signalling pathway (q-value 0.010) (FIG. 4).

The data show the schizophrenia associated vesicular miRNA have dissimilar seed sequences and as they are packaged together in a relatively homogeneous subtype of EV's (MAP1B expressing EV's), this suggests their co-targeting potential is strong, therefore may have a pronounced effect on localised gene expression. Among the eleven differentially expressed miRNA in schizophrenia subjects with cognitive deficit, none of which share the same seed sequence (Table 12), three transcripts are targets of eight of these miRNA. TNFRSF13C (also known as BAFFR) has well established roles in B cell function and B lymphocyte lineages are enriched for schizophrenia associated loci (Ripke et al., 2014). Importantly, TNFRSF13C also has clear nervous system roles; it has been shown to function as a neurotrophic factor by promoting neuronal cell survival (Tada et al., 2013) and is upregulated in temporal neocortex of patients with epilepsy (Ma et al., 2017). TRMT9B (also known as KIAA1456) is brain enriched and the sixth most differentially expressed in dorsolateral prefrontal cortex of schizophrenia subjects (Abed-Esfahani et al., 2021). CNKSR3, a scaffold protein, is significantly down regulated in human induced pluripotent stem cell neurons from schizophrenia subjects (Brennand et al., 2011) and is regulated by the transcription factor TCF7L2, a schizophrenia risk gene (Lipiec et al., 2020). It is not known whether the products of these genes function together in pathways relevant to schizophrenia and cognition, but the strong co-targeting potential of the cognitive deficit associated miRNA suggests profound consequences for the neurobiology of schizophrenia and cognitive function.

TABLE 12
Dysregulated miRNA in cognitive deficit subtype
of schizophrenia have diverse seed sequences.

| | |
|---|---|
| CDvCO up regulated miRNA | Seed sequence |

>hsa-miR-1246 MIMAT0005898

| | |
|---|---|
| AAUGGAUUUUUGGAGCAGG | AUGGAUU |
| (SEQ ID NO: 1) | (SEQ ID NO: 52) |

>hsa-miR-5100 MIMAT0022259

| | |
|---|---|
| UUCAGAUCCCAGCGGUGCCUCU | UCAGAUC |
| (SEQ ID NO: 3) | (SEQ ID NO: 53) |

>hsa-miR-3178 MIMAT0015055

| | |
|---|---|
| GGGGCGCGGCCGGAUCG | GGGCGCG |
| (SEQ ID NO: 4) | (SEQ ID NO: 54) |

>hsa-miR-7704 MIMAT0030019

| | |
|---|---|
| CGGGGUCGGCGGCGACGUG | GGGGUCG |
| (SEQ ID NO: 5) | (SEQ ID NO: 55) |

>hsa-miR-203a-3p MIMAT0000264

| | |
|---|---|
| GUGAAAUGUUUAGGACCACUAG | UGAAAUG |
| (SEQ ID NO: 6) | (SEQ ID NO: 56) |

>hsa-miR-4521 MIMAT0019058

| GCUAAGGAAGUCCUGUGCUCAG | CUAAGGA |
|---|---|
| (SEQ ID NO: 7) | (SEQ ID NO: 57) |
| CDvCO down regulated miRNA | Seed sequence |

>hsa-miR-451a MIMAT0001631

| | |
|---|---|
| AAACCGUUACCAUUACUGAGUU | AACCGUU |
| (SEQ ID NO: 8) | (SEQ ID NO: 58) |

>hsa-miR-92a-3p MIMAT0000092

| | |
|---|---|
| UAUUGCACUUGUCCCGGCCUGU | AUUGCAC |
| (SEQ ID NO: 9) | (SEQ ID NO: 59) |

>hsa-miR-486-5p MIMAT0002177

| | |
|---|---|
| UCCUGUACUGAGCUGCCCCGAG | CCUGUAC |
| (SEQ ID NO: 2) | (SEQ ID NO: 60) |

>hsa-miR-484 MIMAT0002174

| | |
|---|---|
| UCAGGCUCAGUCCCCUCCCGAU | CAGGCUC |
| (SEQ ID NO: 10) | (SEQ ID NO: 61) |

>hsa-miR-93-5p MIMAT0000093

| | |
|---|---|
| CAAAGUGCUGUUCGUGCAGGUAG | AAAGUGC |
| (SEQ ID NO: 11) | (SEQ ID NO: 62) |

CD = Cognitive deficit subtype schizophrenia, CO = Healthy control

Early onset (EOS) and treatment resistant schizophrenia (TRS) are associated with severe disease, worse outcomes and poor prognosis. To investigate if there is a molecular signature of severe disease, participants were examined for TRS (proxied by clozapine use) and EOS (onset before 18 years of age). Participants (n=477) are the same as reported in the substantive analysis. The present inventors first asked if SZ cognitive subtypes (CD and CS) were disproportionately represented in TRS or EOS. While there was no significant representation of cognitive subtypes for EOS, the present inventors did observe that individuals with CD SZ were more likely than those with CS to be also designated TRS (Fisher's exact test, P=0.0003, odds ratio=4.0; 95% confidence interval 1.8-9.8).

The present inventors examined cases only for neuronal miRNA differences in TRS versus non-TRS (n=221, 42 with TRS, 179 with non-TRS) and in EOS versus non-EOS (n=221, 31 with EOS, 190 with non-EOS). The analysis was repeated with non-psychiatric controls (CO) included (n=477, 42 with TRS, 435 with non-TRS, 31 with EOS, 446 with non-EOS).

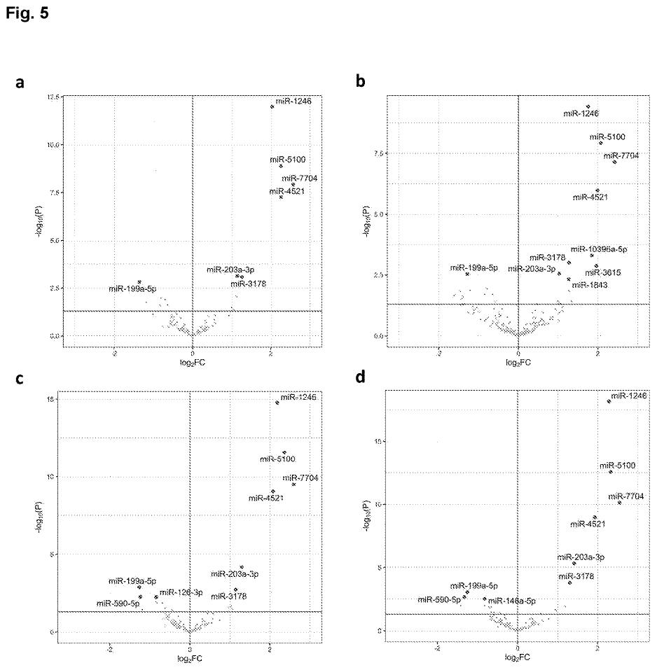

In the cases only analysis (CD and CS), the present inventors identified seven differentially expressed miRNA including six that were upregulated and one downregulated, in TRS compared to the non-TRS group (FIG. 5a, Table 13). With the exception of miR-199a-5p, the miRNA observed to be dysregulated in the TRS group was very similar to what was observed in the substantive analyses. Specifically, miR-5100, miR-7704 and miR-4521 were also increased in cognitive deficit subtype schizophrenia (CD) compared to cognitively spared subtype schizophrenia (CS). In addition to the above, miR-203a-3p and miR-3178 were also increased in CD compared to non-psychiatric controls (CO). Finally, miR-1246 was also increased in schizophrenia subjects (SZ) compared to CO and CD compared to both CO and CS. To gain functional insight into the miRNA differentially expressed in TRS, analysis of the predicted targets revealed they are enriched for the process of neuron development and the cell component synapse (Table 14).

TABLE 13
miRNA differentially expressed in treatment resistant schizophrenia
subjects compared to treatment responsive counterparts.

| | | |
|---|---|---|
| | log₂ fold change (95% | |
| mature miRNA name | confidence interval) | false discovery rate |

Treatment resistant schizophrenia compared to non-treatment
resistant schizophrenia (cases only, 105 miRNA)

| | | |
|---|---|---|
| hsa-miR-1246 | 2.038 [1.48, 2.60] | 1.08E−10 |
| (SEQ ID NO: 1) | | |
| hsa-miR-5100 | 2.259 [1.53, 2.99] | 6.64E−08 |
| (SEQ ID NO: 3) | | |
| hsa-miR-7704 | 2.566 [1.68, 3.45] | 4.25E−07 |
| (SEQ ID NO: 5) | | |
| hsa-miR-4521 | 2.258 [1.44, 3.07] | 1.42E−06 |
| (SEQ ID NO: 7) | | |
| hsa-miR-203a-3p | 1.135 [0.48, 1.79] | 1.48E−02 |
| (SEQ ID NO: 6) | | |
| hsa-miR-3178 | 1.252 [0.52, 1.99] | 1.48E−02 |
| (SEQ ID NO: 4) | | |
| hsa-miR-199a-5p | −1.356 [−2.19, −0.52] | 2.28E−02 |
| (SEQ ID NO: 18) | | |

TABLE 14
Gene and disease ontologies enriched for genes targeted by miRNA
that are dysregulated in subjects with treatment resistant schizophrenia
compared to treatment responsive counterparts.

| | | | | | |
|---|---|---|---|---|---|
| Ontology ID | Ontology name | n | % | p-value | Bonferroni |

GO: Biological process

| | | | | | |
|---|---|---|---|---|---|
| GO: 0048666 | neuron development | 487 | 11.5 | 2.542E−20 | 3.095E−16 |
| GO: 0007417 | central nervous system | 480 | 11.3 | 1.373E−17 | 1.671E−13 |
| | development | | | | |
| GO: 0000902 | cell morphogenesis | 413 | 9.7 | 7.167E−17 | 8.726E−13 |
| GO: 0031175 | neuron projection | 412 | 9.7 | 9.440E−17 | 1.149E−12 |
| | development | | | | |
| GO: 0060322 | head development | 396 | 9.3 | 1.161E−16 | 1.414E−12 |

GO: Cellular component

| | | | | | |
|---|---|---|---|---|---|
| GO: 0005794 | golgi apparatus | 506 | 11.9 | 1.053E−17 | 1.541E−14 |
| GO: 0045202 | synapse | 475 | 11.2 | 1.131E−17 | 1.655E−14 |
| GO: 0098791 | golgi apparatus | 299 | 7.0 | 8.813E−16 | 1.289E−12 |
| | subcompartment | | | | |
| GO: 0031984 | organelle subcompartment | 515 | 12.1 | 6.374E−13 | 9.325E−10 |
| GO: 0000139 | golgi membrane | 249 | 5.9 | 8.758E−13 | 1.281E−9 |

GO: Molecular function

| | | | | | |
|---|---|---|---|---|---|
| GO: 0043565 | sequence-specific DNA binding | 536 | 12.6 | 1.415E−17 | 4.067E−14 |
| GO: 0003700 | DNA-binding transcription | 444 | 10.5 | 1.292E−15 | 3.714E−12 |
| | factor activity | | | | |
| GO: 0001067 | transcription regulatory region | 492 | 11.6 | 1.392E−15 | 4.002E−12 |
| | nucleic acid binding | | | | |
| GO: 1990837 | sequence-specific double- | 501 | 11.8 | 2.414E−15 | 6.939E−12 |
| | stranded DNA binding | | | | |
| GO: 0000976 | transcription cis-regulatory | 488 | 11.5 | 5.046E−15 | 1.450E−11 |
| | region binding | | | | |

n = number of annotated genes from input list, % = n/total number of genes from input list (total 4243).

To expand the identification of potentially informative neuronal origin miRNA with respect to TRS, the present inventors repeated the analysis with a larger set of miRNA (by reducing the sample size in which detection is considered robust). In addition to the findings described above, they also observed miR-10396a-5p, miR-1843 and miR-3615 to be increased in TRS (FIG. 5b). Similar to miR-1246, miR-1843 was also increased in the substantive analyses but did not reach statistical significance in those comparisons (p-value>0.05).

To further increase the scope of disease associated molecules, the present inventors repeated the analysis with CO included (as non-TRS). Similar to the above, they identified the same six increased miRNA (miR-1246, miR-5100, miR-7704, miR-4521, miR-203a-3p and miR-3178) with (FIG. 5c, Table 15) and without (FIG. 5d) adjustment for case control status. For miRNA with decreased expression in TRS, they again observed miR-199a-5p, in addition to miR-590-5p, whereas miR-126-3p and miR-146a-5p were decreased in the adjusted and unadjusted analysis, respectively.

TABLE 15
miRNA differentially expressed in treatment resistant
schizophrenia subjects compared to treatment responsive
counterparts and healthy controls.

| | | |
|---|---|---|
| | log₂ fold change (95% | |
| mature miRNA name | confidence interval) | false discovery rate |

Treatment resistant schizophrenia compared to non-treatment resistant
schizophrenia plus controls (cases and controls), adjusted

| | | |
|---|---|---|
| hsa-miR-1246 | 2.185 [1.65, 2.72] | 1.80E−13 |
| (SEQ ID NO: 1) | | |
| hsa-miR-5100 | 2.359 [1.70, 3.02] | 1.40E−10 |
| (SEQ ID NO: 3) | | |
| hsa-miR-7704 | 2.599 [1.79, 3.41] | 1.10E−08 |
| (SEQ ID NO: 5) | | |
| hsa-miR-4521 | 2.082 [1.42, 2.75] | 2.36E−08 |
| (SEQ ID NO: 7) | | |
| hsa-miR-203a-3p | 1.289 [0.65, 1.92] | 1.43E−03 |
| (SEQ ID NO: 6) | | |
| hsa-miR-199a-5p | −1.263 [−2.04, −0.49] | 2.33E−02 |
| (SEQ I DNO: 18) | | |
| hsa-miR-3178 | 1.144 [0.42, 1.86] | 2.77E−02 |
| (SEQ ID NO: 4) | | |
| hsa-miR-590-5p | −1.237 [−2.11, −0.37] | 6.55E−02 |
| (SEQ ID NO: 19) | | |
| hsa-miR-126-3p | −0.840 [−1.43, −0.25] | 6.55E−02 |
| (SEQ ID NO: 20) | | |

There were no differences in miRNA expression between EOS and non-EOS for both cases only and the full cohort analyses, although reduced expression of miR-3175 and miR-223-3p in cases only EOS were nominally significant (p-value<0.05, data not shown).

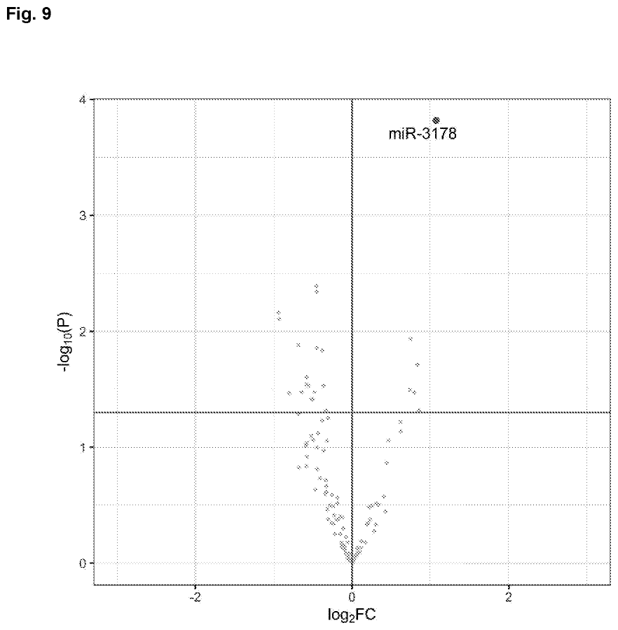

Depressive symptoms in schizophrenia are common and given that depression is a risk factor for suicide and suicide rates are much higher in schizophrenia subjects than the general population, the present inventors examined participants for depression (proxied by use of antidepressants). Participants (n=477) are the same as reported in the substantive analysis. Analysis of the full cohort revealed increased expression of miR-3178 (FDR<0.05) in schizophrenia subjects currently taking an antidepressant (n=60), compared to subjects not taking an antidepressant (n=417), (Table 16, FIG. 9). In the cases only analysis, differential expression of miRNA was not observed, although seven molecules with reduced and five molecules with elevated expression were nominally significant (p-value<0.05),

### (Table 16).

TABLE 16
miRNA differentially expressed in schizophrenia
subjects with depression

| | | |
|---|---|---|
| | log₂ fold change (95% | |
| mature miRNA name | confidence interval) | false discovery rate |

Schizophrenia with depression compared to non-depressed
schizophrenia plus controls (cases and controls), unadjusted

| | | |
|---|---|---|
| hsa-miR-3178 | 1.071 [0.52, 1.63] | 1.59E−02 |
| (SEQ ID NO: 4) | | |

Schizophrenia with depression compared to
non-depressed schizophrenia (cases only)

| | | |
|---|---|---|
| hsa-miR-451a | −0.851 [−1.52, −0.19] | 0.42 |
| (SEQ ID NO: 8) | | |
| hsa-miR-10b-5p | 0.960 [0.17, 1.75] | 0.42 |
| (SEQ ID NO: 21) | | |
| hsa-miR-16-5p | −0.670 [−1.22, −0.12] | 0.42 |
| (SEQ ID NO: 24) | | |
| hsa-miR-7704 | 0.942 [0.13, 1.76] | 0.42 |
| (SEQ ID NO: 5) | | |
| hsa-miR-19b-3p | −0.377 [−0.71, −0.04] | 0.42 |
| (SEQ ID NO: 25) | | |
| hsa-miR-23a-3p | −0.386 [−0.74, −0.03] | 0.42 |
| (SEQ ID NO: 26) | | |
| hsa-miR-20a-5p | −0.861 [−1.66, −0.06] | 0.42 |
| (SEQ ID NO: 27) | | |
| hsa-miR-3178 | 0.748 [0.05, 1.45] | 0.42 |
| (SEQ ID NO: 4) | | |
| hsa-miR-3648 | 0.747 [0.05, 1.45] | 0.42 |
| (SEQ ID NO: 22) | | |
| hsa-miR-1843 | 0.790 [0.03, 1.55] | 0.42 |
| (SEQ ID NO: 23) | | |
| hsa-miR-21-5p | −0.581 [−1.15, −0.02] | 0.42 |
| (SEQ ID NO: 28) | | |
| hsa-let-7a-5p | −0.363 [−0.72, −0.00] | 0.42 |
| (SEQ ID NO: 29) | | |

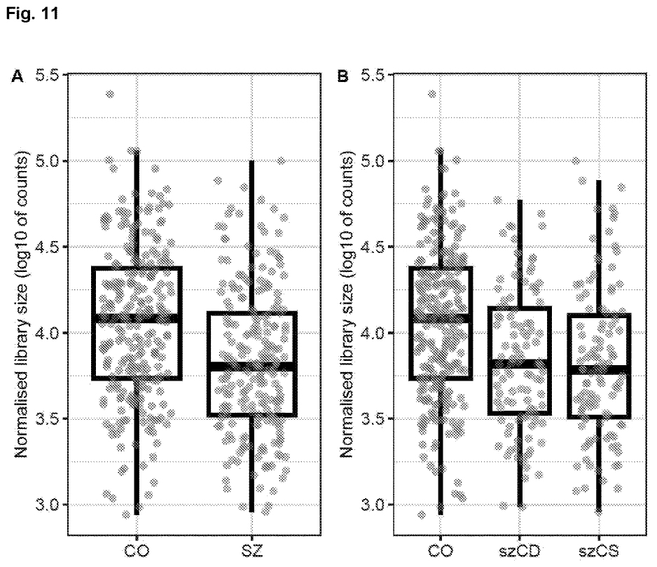

Disease associated alterations in circulating neuronal origin EVs provide a read-out of synaptic plasticity disorders. The present inventors compared the quantity of neuronal origin EVs, by virtue of their miRNA cargo, recovered from serum of subjects with schizophrenia (SZ, n=221) and comparison subjects (CO, n=256) (FIG. 11). The present inventors identified reduced circulating miRNA in SZ compared to CO (Wilcoxon rank sum test, P=4.385e-08) which equates to a log 10 fold decrease of about 0.282 (95% CI: −0.380, −0.185) (FIG. 11A). When partitioning SZ by cognition, schizophrenia subjects with severe cognitive deficits (szCD, n=111) and schizophrenia subjects with spared cognition (szCS, n=110), each demonstrate reduced circulating miRNA compared to non-psychiatric comparison subjects (szCD: P=2.5e-05; Wilcoxon rank sum test, szCS: P=1.8e-05; Wilcoxon rank sum test) but not compared to each other (szCD vs. szCS: P=0.6; Wilcoxon rank sum test) (FIG. 11B).

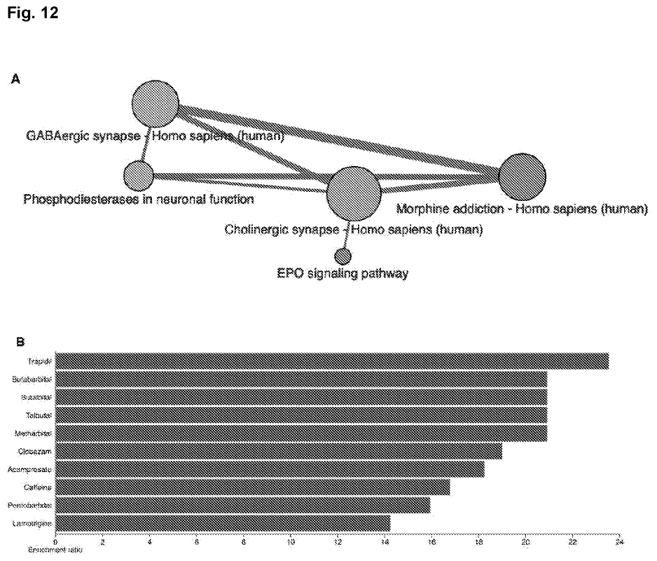

Patient stratification for the purposes of biologically informed treatment strategies is one aspect of implementing precision medicine. Here, the present inventors demonstrate that schizophrenia subjects with severe cognitive deficits have a neuronal miRNA profile that indicates potential treatment options to improve cognitive performance. This represents an urgent unmet need given that cognitive impairments in schizophrenia are a strong predictor of poor outcomes and are not adequately treated with current pharmacotherapies. Analysis of genes targeted by miRNA altered in szCD identified enrichment in signaling pathways important to synaptic plasticity, including Brain-derived neurotrophic factor (BDNF) signaling, CAMP signaling and splicing factor NOVA regulated synaptic proteins. Additionally, the enriched pathways also include Erythropoietin (EPO) signaling, Morphine addiction, Cholinergic synapse, Phosphodiesterases in neuronal function and GABAergic synapse (FIG. 12A). Notably, these five pathways collectively contain 117 miRNA target genes, many of which encode for proteins that are drug targets of approved medications (77 of 117, 66%) (FIG. 12B). Importantly, none of medications listed (FIG. 12B) were reported for the ASRB participants included in this work.

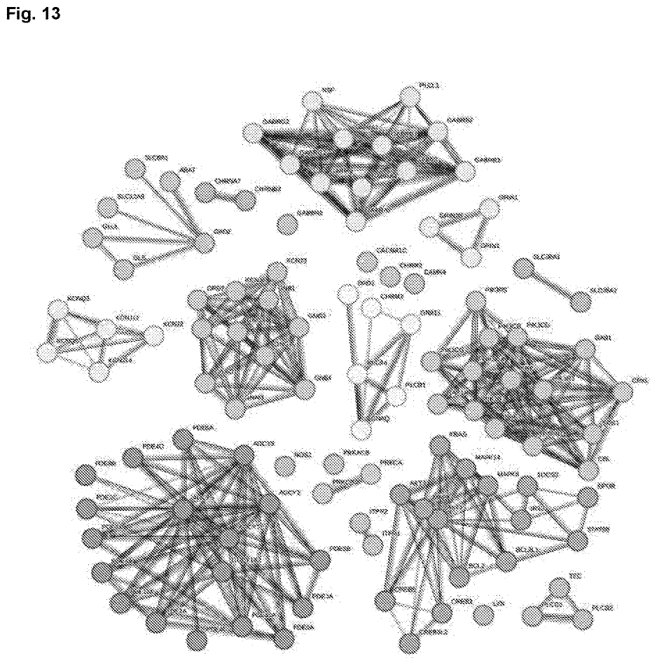

Most of the identified drugs (FIG. 12B) target GABA subunits, for example butabarbital, pentobarbital and clobazam. However, clustering of the pathway genes by protein-protein interaction networks identifies phosphodiesterases in neuronal function genes as the largest cluster (FIG. 13), suggesting potential therapeutic application for phosphodiesterase inhibitors, such as trapidil (FIG. 12B), particularly for szCD. Additionally, the second and third largest clusters are composed of EPO signaling and cholinergic synapse pathway genes.

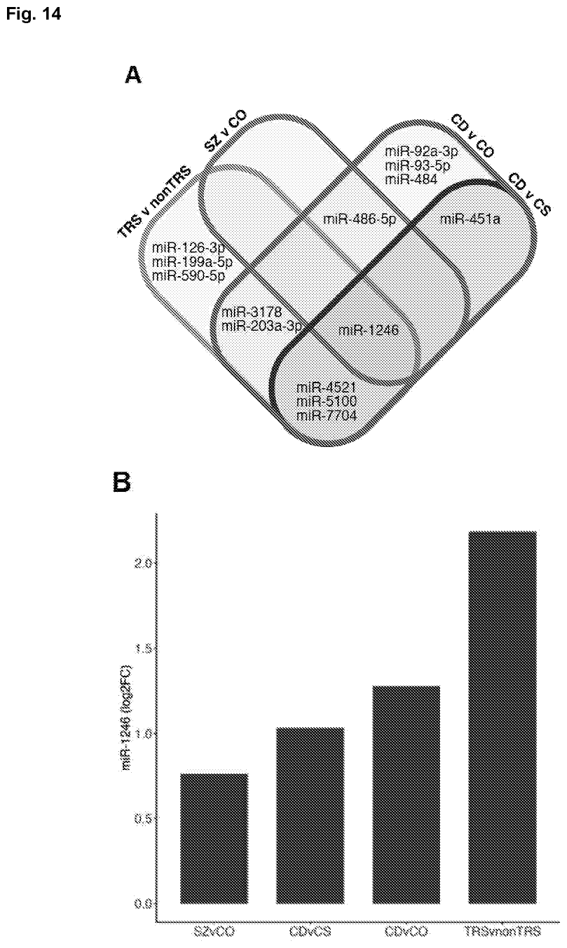

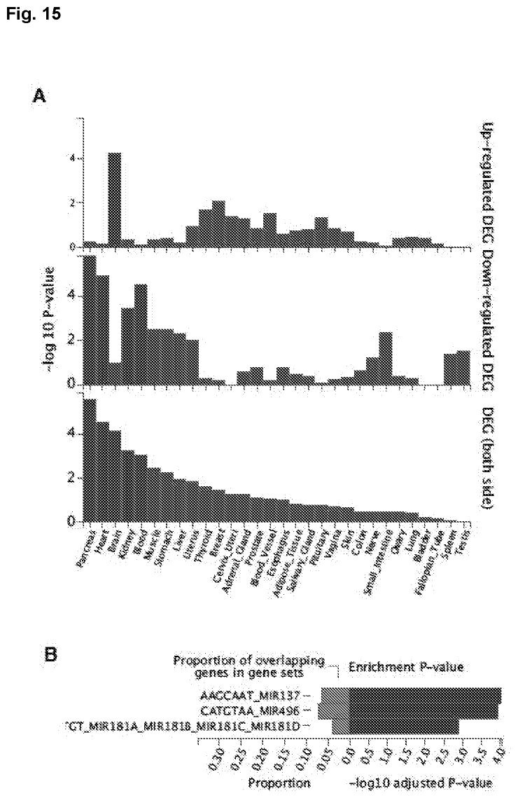

The present inventors observed several overlapping miRNA when identifying expression differences between groups (FIG. 14A). miR-1246 was the most consistently differentially expressed, showing increased expression across all groups (FIG. 14B). High confidence predicted targets of miR-1246 are brain enriched and overlap with miR-137 targets (FIGS. 15, A and B). The second set of miRNA (miR-4521, miR-5100 and miR-7704) were consistent across three groups, followed miR-203a-3p, miR-3178, miR-451a and miR-486-5p which were consistent across two groups.

### Discussion

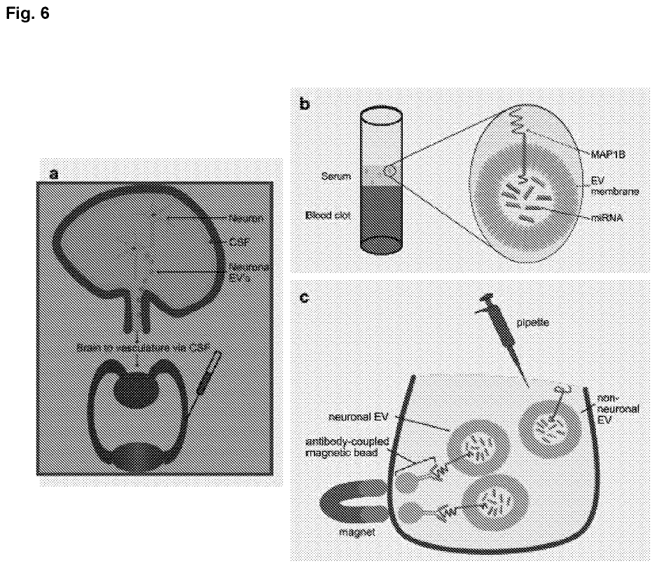

In this study, the present inventors explored the utility of MAP1B-immuno-fractionated serum EV's as a proxy of brain associated miRNA expression (FIG. 6) and used this insight to explore the difference in a large serum cohort of individuals with schizophrenia and non-psychiatric controls. They demonstrate that enrichment for neuronal origin serum EV's is both viable and informative for revealing a profile of miRNA species consistently observed across samples from an otherwise relatively inaccessible neural tissue. This profile was enriched for miR-17~92 cluster molecules, encoded as a polycistronic miRNA gene on chromosome thirteen (with paralogs on chromosomes seven and X). This cluster is highly conserved among vertebrates and is processed into six mature miRNA (miR-17, miR-18a, miR-19a, miR-19b-1, miR-20a, miR-92a), which are expressed in a variety of tissues, including the brain (Ludwig et al., 2016). The miR-17~92 cluster molecules play roles in neuronal differentiation (Beveridge et al., 2009), axonal outgrowth (Zhang et al., 2013) and when overexpressed in mesenchymal stem cell exosomes, they promote neurological recovery from stroke (Xin et al., 2017). Additionally, clustering analysis reveals miR-19a and miR-19b (members of miR-17~92 cluster) are the most strongly correlated molecules (positively) with the first principal component (dimension one), explaining the largest proportion of variation in the data (9.7%). Interestingly, miR-19a and miR-19b have been shown to direct the migration of adult-born neural progenitor cells and regulate dendritic morphogenesis of adult born neurons in the dentate gyrus of the hippocampus (Han et al., 2016). The present inventors also show that the brain associated miRNA profile from all subjects shows tissue specificity for the brain (cerebellum) and is enriched for let-7 and miR-29 families, molecules known to be expressed in mammalian brain (Landgraf et al., 2007; Pena et al., 2009). Taken together, the present inventors show that serum EV's enriched for neuronal origin provide a readout of the brain associated miRNA regulatory environment in living subjects.

The present inventors investigated if alterations in the neuronal miRNA profile could be detected in psychiatric illness and found schizophrenia subjects do indeed show dysregulated expression of two miRNA, miR-486-5p and miR-1246. These two miRNA are brain enriched (Panwar, Omenn and Guan, 2017), predicted to target genes that function in synapse organisation and are enriched at the postsynaptic membrane. Additionally, miR-486-5p from L1CAM enriched plasma EV's was recently reported to be associated with antidepressant treatment response in MDD (Saeedi et al., 2021), although L1CAM as a neuronal EV marker may not be ideal, at least in proteomics studies (Norman et al., 2021). There is a broad spectrum in the symptom profile of schizophrenia, suggesting biological subtypes may be more meaningful than diagnostic categories for determining mechanisms of dysfunction and improving outcomes. Cognitive symptoms are a core feature of schizophrenia and a key predictor of poor outcome. Given that miRNA play important roles in processes that subserve cognition (synaptic plasticity, learning and memory), the present inventors investigated the neuronal miRNA profile based on cognitive subtypes (Green et al., 2013). Indeed, stratification by cognition revealed subjects with more severe cognitive deficit displayed an even greater degree of dysregulation in miRNA expression than was seen in cognitively spared counterparts, suggesting the circulating neuronal miRNA profile may be informative for cognitive domains of illness. This profile contained brain enriched miRNA and was mostly characterised by increased expression, suggesting the cellular compartment of origin contains an abundance of these regulatory molecules. Furthermore, the predicted targets of dysregulated miRNA are enriched in synaptic plasticity related terms such as neurogenesis, while also showing enrichment for schizophrenia associated common variation. To the best of their knowledge, miRNA analysis of fractionated neuronal-origin EV's from the peripheral circulation of schizophrenia subjects has not been previously reported.

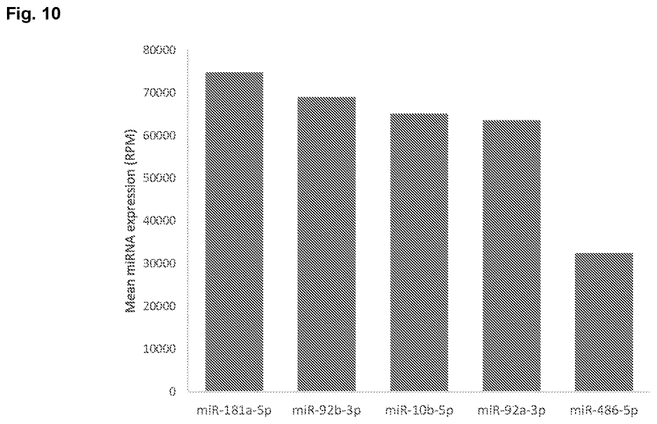

The findings of schizophrenia-associated alterations in miRNA abundance demonstrate that neuronal-origin enrichment of serum EV's reveals disease associated miRNA profiles from disease relevant cell types (neurons). As this may represent a niche of human brain miRNA in the peripheral circulation, the present inventors speculated that these miRNA would have been previously observed in human CNS tissues such as cerebrospinal fluid, prefrontal cortex, prefrontal cortex EV's, superior temporal gyrus and amygdala. They observed that the vast majority of differentially expressed miRNA (31 of 33 unique miRNA) have been reported as expressed in CNS tissues and eleven of these miRNA have been previously associated with schizophrenia (Table 17). Furthermore, they also queried a brain dataset that specifically analysed miRNA (Panwar, Omenn and Guan, 2017) and ranked those mature miRNA based on the reported mean RPM expression. They found that two of the most dysregulated miRNA identified here (hsa-miR-486-5p and hsa-miR-92a-3p) are among the top five expressed in the human brain (FIG. 10). These results support the objective to capture brain expressed miRNA from the peripheral circulation, not unlike a recent report for MDD (Saeedi et al., 2021). Moreover, just as bulk tissue analysis can dilute molecules with relatively low abundance leading to loss of biological information, their enrichment for relatively homogenous EV's rather than the global population, has revealed disease associated miRNA that may otherwise be missed. Also, in contrast to bulk analysis of circulating EV's, the function of these miRNA can be interpreted based on the cellular context, in this case miRNA regulation of neuronal gene expression. For example, miR-7704 is expressed at low copy number in the human brain (Panwar, Omenn and Guan, 2017) and this likely contributes to a lack of differential expression in bulk brain tissue analyses, whereas in this targeted analysis the signal to noise ratio is improved and miR-7704 is found to be robustly detected and significantly upregulated. Thus, this novel approach to interrogating miRNA dysregulation in an otherwise relatively inaccessible neural tissue, is highly relevant to psychiatric disorders and thus increases the scope of disease associated molecules, an important advance given the complexity and heterogeneity of psychiatric disorders.

TABLE 17
Neuronal origin miRNA from serum EV's differentially expressed
in schizophrenia subjects and expressed in human CNS tissues.

| miRNA from | Differential | | Previously reported as |
|---|---|---|---|
| neuronal origin | expression by | Previously reported as human | human CNS expressed in |
| serum EV's | group | CNS expressed | schizophrenia |
| hsa-miR-1246 | SZ v CO | Brain, region unspecified [7] | N/A |
| | CD v CO | | |
| | CD v CS | | |
| | TRS v nonTRS | | |
| hsa-miR-203a-3p | SZ v CO | Brain, region unspecified [7] | N/A |
| | CD v CO | PFC_BA9_EV's [8] | No change |
| | TRS v nonTRS | CSF [9] | Not reported |
| hsa-miR-150-5p | SZ v CO | Brain, region unspecified [7] | N/A |
| | | PFC_BA9_EV's [8] | Decreased |
| | | STG_BA42 [10] | Decreased |
| hsa-miR-92a-3p | SZ v CO | Brain, region unspecified [7] | N/A |
| | CD v CO | PFC_BA9_EV's [8] | Increased* |
| | CD v CS | PFC_BA9 [11] | Decreased* |
| hsa-miR-3178 | SZ v CO | Brain, region unspecified [7] | N/A |
| | CD v CO | | |
| | CD v CS | | |
| | TRS v nonTRS | | |
| | Dep v nonDep | | |
| hsa-miR-5100 | SZ v CO | CSF [12] | N/A |
| | CD v CO | | |
| | CD v CS | STG_BA22 [13] | N/A |
| | TRS v nonTRS | | |
| hsa-miR-486-5p | SZ v CO | Brain, region unspecified [7] | N/A |
| | CD v CO | PFC_BA9_EV's [8] | No change |
| | CS v CO | CSF_EV's [14] | Decreased* |
| hsa-miR-146a-5p | SZ v CO | Brain, region unspecified [7] | N/A |
| | CD v CO | PFC_BA9_EV's [8] | Increased |
| | | CSF [15] | N/A |
| | | CSF [16] | N/A |
| | | CSF [9] | Not reported |
| | | STG_BA22 [13] | N/A |
| hsa-miR-425-5p | SZ v CO | Brain, region unspecified [7] | N/A |
| | CS v CO | PFC_BA9_EV's [8] | Increased |
| | | PFC_BA9 [17] | Decreased |
| hsa-miR-484 | SZ v CO | Brain, region unspecified [7] | N/A |
| | CD v CO | PFC_BA9_EV's [8] | Decreased |
| | | CSF [16] | N/A |
| hsa-miR-26b-5p | SZ v CO | Brain, region unspecified [7] | N/A |
| | CD v CO | PFC_BA9_EV's [8] | Decreased* |
| | | PFC_BA9 [11] | Decreased* |
| | | STG_BA22 [18] | Increased* |
| | | STG_BA22 [13] | N/A |
| | | CSF [16] | N/A |
| hsa-miR-93-5p | SZ v CO | Brain, region unspecified [7] | N/A |
| | CD v CO | PFC_BA9_EV's [8] | Increased* |
| hsa-miR-451a | CD v CO | PFC_BA9_EV's [8] | Increased |
| | CD v CS | Amygdala [19] | Increased* |
| | SZ v CO | CSF_EV's [20] | Increased* |
| | | PFC_BA10 [21] | N/A |
| | | CSF [12] | N/A |
| | | STG_BA22 [13] | N/A |
| hsa-miR-7704 | CD v CO | CSF [12] | N/A |
| | CD v CS | | |
| | TRS v nonTRS | | |
| hsa-miR-4521 | CD v CO | Temporal lobe cortex [22] | N/A |
| | CD v CS | CSF [23] | N/A |
| | TRS v nonTRS | Cerebellum [24] | N/A |
| hsa-miR-19a-3p | CD v CS | STG_BA22 [18] | Increased* |
| | | PFC_BA9 [18] | Increased* |
| | | PFC_BA9_EV's [8] | No change |
| | | CSF [9] | Not reported |
| | | PFC_BA10 (Mor et al 2015) | N/A |
| hsa-miR-324-5p | CD v CS | PFC_BA9 [25] | N/A |
| | CS v CO | PFC_BA9_EV's [8] | No change |
| hsa-miR-12136 | SZ v CO | N/A | N/A |
| | CS v CO | | |
| hsa-miR-146b-5p | SZ v CO | Brain, region unspecified [7] | N/A |
| | CD v CO | PFC_BA9_EV's [8] | No change |
| | CS v CO | CSF [15] | N/A |
| | | CSF [16] | N/A |
| | | CSF [9] | Not reported |
| hsa-miR-328-3p | SZ v CO | Brain, region unspecified [7] | N/A |
| | | PFC_BA9_EV's [8] | Decreased |
| | | PFC_BA46 [26] | Increased* |
| hsa-miR-335-5p | CD v CO | Brain, region unspecified [7] | N/A |
| | | PFC_BA9_EV's [8] | No change |
| | | STG_BA22 [18] | Increased* |
| hsa-miR-144-3p | SZ v CO | PFC_BA9_EV's [8] | No change |
| | CD v CO | Amygdala [19] | Increased* |
| | | PFC_BA10 (Mor et al 2015) | N/A |
| hsa-miR-145-5p | CD v CO | PFC_BA9_EV's [8] | No change |
| | | PFC_BA46 [27] | Increased^{‡} |
| | | STG_BA22 [13] | N/A |
| hsa-let-7d-3p | SZ v CO | Brain, region unspecified [7] | N/A |
| | CS v CO | | |
| hsa-let-7i-5p | CD v CO | PFC_BA9_EV's [8] | Increased |
| | | CSF [28] | N/A |
| hsa-miR-20a-5p | CD v CO | PFC_BA9_EV's [8] | No change |
| | | STG_BA22 [18] | Increased* |
| | | PFC_BA9 [18] | Increased* |
| | | CSF [9] | Not reported |
| hsa-miR-423-3p | CD v CO | PFC_BA9_EV's [8] | Decreased |
| hsa-miR-590-5p | CD v CO | CSF [16] | N/A |
| | | CSF [9] | Not reported |
| hsa-miR-199a-5p | TRS v nonTRS | CSF [1] | N/A |
| | | PFC_BA9_EV's [8] | No change |
| | | Brain, region unspecified [1] | N/A |
| hsa-miR-142-5p | CD v CO | PFC_BA9_EV's [8] | No change |
| | CD v CS | PFC_BA10 [21] | N/A |
| | | PFC_BA10 [21] | N/A |
| | | CSF [9] | Not reported |
| hsa-miR-301a-3p | CD v CS | PFC_BA9_EV's [8] | Decreased* |
| | | PFC_BA9 [17] | Increased |
| | | PFC_BA9 [25] | N/A |
| | | CSF_EV's [29] | N/A |
| hsa-miR-29a-3p | CD v CS | PFC_BA9_EV's [8] | Decreased |
| | | PFC_BA9 [11] | Decreased |
| | | PFC_BA46 [27] | Decreased^{‡} |
| | | CSF [9] | Not reported |
| | | STG_BA22 [13] | N/A |
| hsa-miR-26a-5p | SZ v CO | PFC_BA9_EV's [8] | No change |
| | CS v CO | CSF_EV's [29] | N/A |
| | | Substantia nigra [30] | N/A |
| | | STG_BA22 [13] | N/A |
| hsa-miR-4485-5p | CD v CS | N/A | N/A |

SZ = Schizophrenia, CD = Cognitive deficit subtype schizophrenia, CS = Cognitive spared subtype schizophrenia, CO = Control, Dep = depression, TRS = treatment resistant schizophrenia, BA = Brodmann's area, CSF = Cerebrospinal fluid, EV's = Extracellular vesicles, PFC = Prefrontal cortex, STG = Superior temporal gyrus.
Groups in bold font represent significant differential expression (FDR < 0.1) and normal font represent nominally significant differential expression (p-value < 0.05) for that miRNA in the current study.
*statistically significant difference in schizophrenia observed by others.
^{‡}Differential expression was observed in bipolar disorder for hsa-miR-145-5p and hsa-miR-29a-3p.

Differential expression of brain-expressed miRNA has been identified in schizophrenia previously and several of these miRNA were also identified in the current study. It is difficult to compare the findings with others given the unique approach used here, namely miRNA profiling of neuronal-origin EV's, whereas others have used bulk tissue homogenates which likely dilute low abundance molecules. Also, the present inventors analysed cognitive subtypes whereas others have not. Nevertheless, they observed significant differential expression of miR-92a-3p and miR-93-5p in cognitive deficit subjects compared to non-psychiatric controls. These miRNA have been identified as dysregulated in EV's from prefrontal cortex of schizophrenia subjects, but cognitive subtypes were not investigated (Banigan et al., 2013). miR-92a-3p was also identified as dysregulated in the prefrontal cortex of schizophrenia subjects, but neither EV's nor cognitive subtypes were investigated (Perkins et al., 2007). In the current study, the present observed significant differential expression of miR-451a in cognitive deficit subjects, compared to both non-psychiatric controls and cognitive spared subjects, and this miRNA has previously been identified as dysregulated in the amygdala of schizophrenia subjects, but neither EV's nor cognitive subtypes were investigated (Liu et al., 2018). Finally, in the current study, the present inventors observed significant differential expression of miR-7704 in cognitive deficit subjects, compared to both non-psychiatric controls and cognitive spared subjects, and this miRNA has previously been identified as dysregulated in schizophrenia neurons generated from induced pluripotent stem cells, but neither EV's nor cognitive subtypes were investigated (Zhao et al., 2015).

In the context of EV's, reports indicate specific miRNA are incorporated in EV's rather than a random selection of the cellular pool of miRNA (Guduric-Fuchs et al., 2012). While it is not expected that all cell types under all conditions select the same miRNA for EV loading, there is a general consensus for EV associated miRNA in some instances. For example, miR-451a is enriched in EV's relative to cells (Guduric-Fuchs et al., 2012), robustly detected in plasma EV's (Gamez-Valero et al., 2019), serum EV's (Ebrahimkhani et al., 2017), post-mortem prefrontal cortex EV's (Banigan et al., 2013) and CSF EV's (Gallego et al., 2018b). It has been suggested that the frequent reports for EV localisation of miR-451a may be related to the noncanonical processing of this miRNA, specifically Dicer-independent, AGO2-dependent slicing (Yang et al., 2010). Furthermore, it appears miR-451a is not the only EV-enriched miRNA that is dependent on AGO2 because miR-486 is also EV enriched and AGO2 dependent for processing (Yang et al., 2010; Guduric-Fuchs et al., 2012; Jee et al., 2018). Given that the current study found miR-451a and miR-486-5p were significantly decreased in cognitive deficit compared to non-psychiatric controls and they each show degrees of AGO2 dependency, this may be indicative of perturbations that manifest at the level of AGO2 functionality. Furthermore, AGO2 itself has been shown to be secreted in EV's (Gibbings et al., 2009; Mckenzie et al., 2016) and mediates miRNA sorting to EV's (Mckenzie et al., 2016). Taken together, these lines of evidence suggest that in schizophrenia, and particularly cognitive deficit subjects, there is a link between sorting of miRNA to EV's and miRNA biogenesis (at the level of AGO2). Interestingly, miRNA biogenesis genes (Drosha, DGCR8 and Dicer) have previously been observed to be dysregulated in schizophrenia (Beveridge et al., 2010; Santarelli et al., 2011).

In the context of specific miRNA identified in the current study, some interesting insights emerge. High expression of miR-451a in erythrocytes has been extensively reported (review (Wang, Wu and Yu, 2019)), yet this miRNA is frequently observed in non-erythroid tissues (as described above) and the presence of this miRNA was assumed by others to be due to contamination. There is however, mounting evidence that miR-451a is also important in neuronal function; it is dysregulated in CNS tissues from a variety of neurodegenerative and mental disorders including prion disease (Boese et al., 2016), epilepsy (Raoof et al., 2017), Alzheimer's disease (Mckeever et al., 2018) and schizophrenia (Banigan et al., 2013; Gallego et al., 2018a; Liu et al., 2018). Recently, endogenous expression of miR-451a was shown to increase with neuronal differentiation and was detected in adult mouse hippocampus, especially the subgranular zone and hilus of the dentate gyrus (Trattnig et al., 2018), regions important for adult neurogenesis. In the same study, overexpression of miR-451a induced earlier neuronal differentiation and neurite network formation while knockdown had the opposite effect. Finally, genetic ablation led to subtle differences that ultimately suggested the balance between neurogenesis, migration and differentiation was impaired (Trattnig et al., 2018). There is also support for correlation between miR-451a expression in mouse hippocampus and behavioural phenotypes relevant to schizophrenia, namely exploratory behaviour and learning and memory (Parsons et al., 2008). Early-life stress, a risk factor for schizophrenia, was associated with reduced expression of miR-451a in the rat hippocampus and reversed with chronic fluoxetine treatment (O'Connor et al., 2013). The results reveal a strong signal for miR-451a in schizophrenia subjects with cognitive deficits, more than two fold down regulated compared to both controls and cognitively spared schizophrenia subjects. Given the enrichment for neuronal origin miRNA, the present inventors can interpret the dysregulation of miR-451a in the context of neuronal function. Together with the aforementioned studies and the present results demonstrating enrichment for neurogenesis, miR-451a may play an important role in regulating neurogenesis related expression programs with functional consequences for cognition.

Like miR-451a, miR-486 is also EV enriched (Guduric-Fuchs et al., 2012), undergoes AGO2-dependent slicing (Jee et al., 2018) and is highly expressed in erythrocytes (Jee et al., 2018). As mentioned earlier, miR-486-5p is also highly abundant in human brain (Panwar, Omenn and Guan, 2017). Until recently, a specific role for miR-486-5p in the brain had not been established, but recent work of Dori et al (2020) reported increased neural progenitors at the expense of neurons following inhibition of mature miR-486-5p in mouse lateral cortices (Dori et al., 2020).

Both miR-451a and miR-486-5p are significantly reduced in neuronal origin EV's from schizophrenia subjects with cognitive deficit and this could be interpreted in two ways. Firstly, the reduced EV localisation could arise from depleted cellular levels of these molecules. Secondly, the reduced EV localisation could arise from cellular retention of these miRNA implying that even though they are typically EV-enriched, sorting of these molecules to EV's is impaired. Indirect support for the latter interpretation comes from previous studies demonstrating increased expression of miRNA and miRNA biogenesis genes in schizophrenia postmortem brain (Beveridge et al., 2010; Santarelli et al., 2011). Cellular elevation of these molecules may lead to increased EV localisation of miRNA, perhaps to achieve higher turnover rates in response to cellular elevation (Ghosh et al., 2015). The present results largely support this idea; the majority of differentially expressed miRNA are increased (six of eleven and four of five in schizophrenia subjects with cognitive deficit compared to non-psychiatric controls and cognitively spared, respectively). Under homeostatic conditions, approximately 40% of miRNA duplexes do not produce mature miRNA, and this is posited to ensure a balance in the availability of AGO substrates (miRNA) and free AGO (Reichholf et al., 2019). In a scenario of increased miRNA, this would increase the demand for free AGO proteins due to the increased availability of substrates. The vast majority of miRNA are Dicer processed (Dicer is upregulated in schizophrenia (Beveridge et al., 2010; Santarelli et al., 2011)) and can associate with any of the four AGO proteins for RISC assembly and so the increased demand is distributed across the four AGO proteins. For AGO2 dependent miRNA, especially miR-451a but also miR-486-5p, there is no alternative for AGO2 activity as AGO1, 3 and 4 cannot efficiently slice these duplexes and this may lead to reduced maturation and subsequent reduction in EV localisation, in line with our observation. It is interesting to speculate that miR-451a and miR-486-5p, being uniquely vulnerable to the availability of AGO2, act as a signal for a bottle neck occurring at the RISC assembly stage of miRNA-mediated posttranscriptional regulation.

The present inventors observed several miRNA associated with TRS that were similar to those revealed in the main analysis, except the magnitudes were much greater. For example, while miR-7704 was increased by approximately two-fold in CD compared to CO and CS, it was increased by more than five-fold in TRS compared to non-TRS. This supports the disease severity model of TRS (Howes, Thase and Pillinger, 2021) as opposed to other models which suggest that TRS is neurobiologically distinct from non-TRS (Kim et al., 2017). Interestingly, the expression of miR-7704 was observed to be elevated in neurons generated from induced pluripotent stem cells derived from schizophrenia subjects (Zhao et al., 2015).

The observation of reduced miR-199a-5p, miR-590-5p and miR-126-3p expression in TRS compared to non-TRS is interesting in that neither miR-199a-5p nor miR-126-3p were differentially expressed and miR-590-5p was nominally significant (p<0.05) in the main analysis even though the miRNA and SZ subjects tested were the same. This could be interpreted as reflecting antipsychotic treatment or severe disease. In support of the latter, in exosomes from human prefrontal cortex (Brodmann's area 9) of schizophrenia subjects, miR-199a-5p and miR-126-3p were detected but not differentially expressed, while miR-199a-3p was significantly increased and remained significant after adjusting for medication use which included clozapine (Banigan et al., 2013). While Banigan et al did not specifically test TRS versus non-TRS, the evidence does suggest a disease association rather than a medication effect, at least for miR-199a-3p (Banigan et al., 2013). Conversely, in rodents, miR-199a-5p was increased in the rat medial frontal cortex following haloperidol treatment (Perkins et al., 2007). Given that none of the TRS subjects in our study were receiving haloperidol treatment, this suggests that the differential expression of miR-199a-5p may not be drug specific or dependent. When clozapine was directly tested in a mouse model, miR-199a-5p, miR-590-5p and miR-126-3p were not reported as detected or differentially expressed in whole brain (Santarelli et al., 2013) or in the C6 rat glioma cell line, although miR-199a-3p was reduced in the latter following clozapine treatment (Wang et al., 2020). Finally, plasma miR-126-3p levels were significantly decreased in subjects taking clozapine, compared to healthy control subjects that had never used clozapine (Burns et al. 2020). Importantly, this reduction was suggested by the authors to be a disease related event rather than clozapine induced, because miR-126-3p levels, although differentially expressed, were stable in both controls and cases over a six week period (from commencement of clozapine treatment) (Burns et al., 2020).

The lack of miRNA association with EOS is not unanticipated given it's relatively rare occurrence (Guo et al., 2021), in addition to suggested differences (between EOS and adult onset SZ) in genetic and environmental contributions (Forsyth and Asarnow, 2020), both of which influence miRNA expression. Nevertheless, although decreased miR-3175 was only nominally significant in the current work, it has previously been reported as downregulated in neurons generated from induced pluripotent stem cells from SZ subjects which included childhood onset cases (two of six cases) (Zhao et al., 2015). Likewise, decreased miR-223-3p was only nominally significant in the current work but increased plasma expression of miR-223-3p has been reported for first episode SZ (Zhao et al., 2019). Furthermore, Amoah et al identified increased miR-223-3p expression from postmortem orbitofrontal cortex of SZ subjects (including nine of twenty-nine EOS) and the authors went on to identify this miRNA as enriched in neuronal exosomes (compared to neuronal cellular levels) from primary mouse cortical cultures (Amoah et al., 2019). The authors further demonstrated haloperidol and olanzapine treatment of primary mouse cortical cultures were associated with reduced miR-223-3p in neuronal exosomes compared to vehicle (Amoah et al., 2019). Given that there were no EOS subjects taking haloperidol and only five EOS subjects taking olanzapine in the current work, it is difficult to reconcile these findings. Taken together, alterations in miR-3175 and miR-223-3p, particularly in the context of human neuronal and neuronal EV expression, may serve as indicators of severe disease (proxied by EOS) but larger sample sizes are needed to confirm this.

This work provides evidence that the neuronal origin miRNA profile in serum EV's may have the potential to predict TRS and could support clinical decisions to initiate clozapine treatment at an earlier stage given its advantage for treating resistant disease (Bachmann et al., 2017).

In addition to alterations in cognitive and treatment resistant subtypes of schizophrenia, the present inventors extend those findings to mood disorders, where they observed increased expression of miR-3178 in schizophrenia subjects with depression. Interestingly, HTR1A (5-hydroxytryptamine receptor 1A), a gene encoding a serotonin receptor, is a validated target of hsa-miR-3178 in neuronal cell lines (Wu et al., 2019). Also in a neuronal cell line (SH-SY5Y) hsa-miR-3178 was identified as enriched in neurites relative to the cell body (Goldie et al., 2014), implying a neuron specific function for this miRNA, at least under cell culture conditions. Furthermore, HTR1A shows neuron specific splice variants that are found in human brain but not mouse (Le François et al., 2018). Finally, the spliced variants lose a miR-135 binding site leading to increased stability of HTR1A, while the miR-3178 binding site is retained. It is interesting to speculate that the retention of miR-3178 binding site provides more nuanced control over the stability and translational competence of this receptor. To the best of the present inventors knowledge, miR-3178 has not previously been identified as associated with depression, nor depression in the context of schizophrenia (see a recent review (Gibbons, Sundram and Dean, 2020)).

Previous studies of miRNA have not reported miR-3178 changes with respect to antidepressant medication (Baudry et al., 2010; Launay et al., 2011, Bocchio-Chiavetto et al., 2013, Pan and Liu, 2015; Zhang et al., 2015, Mundalil Vasu et al., 2016, Saeedi et al., 2021), suggesting that the dysregulation observed here is, at least in part, due to psychopathology.

SZ is a disorder of synaptic plasticity (Bartolomeis et al., 2023), with many lines of evidence supporting alterations in the synaptic proteome and transcriptome (Lipska et al., 2006; Hashimoto et al., 2008; Maldonado-Aviles et al., 2009; Gandal et al., 2018). Although MAP1B itself has not been directly linked to SZ, it is plasticity associated, participating in structural and functional synaptic adaptations during development and in the adult brain (Nothias et al., 1996; Eriksson et al., 2010; Gandini et al., 2014; Kim et al., 2014; Bodaleo et al., 2016). miRNA also play a key role in synaptic plasticity via homeostatic and activity driven regulation of gene expression (Cohen et al., 2011; Sambandan et al., 2017; Martins and Schratt 2021). In this context, miRNA biogenesis and turnover are the main determinants of abundance and both processes are regulated by neuronal activity (Krol et al., 2010). Given that a mechanism of miRNA turnover includes loading into EVs (Ghosh et al., 2015; Ghosh et al., 2021) and brain EVs are enriched for MAP1B (Vella et al., 2017; You et al., 2023) and miRNA (Goldie et al., 2014), this suggests that miRNA loaded into MAP1B EVs function as a mechanism to augment the synaptic regulatory environment. Therefore, our observation that neuronal origin serum EV miRNA, collectively, are decreased in SZ suggests this feature is identifying a read-out of altered synaptic function and can be applied to biomarker development.

The enrichment of genes in phosphodiesterases in neuronal function, cholinergic synapse and EPO signaling is intriguing given a resurgence of interest in targeting these pathways. For example, the phosphodiesterase (PDE) inhibitor roflumilast (selective PDE4 inhibitor) was recently shown to improve cognitive performance in schizophrenia subjects (Livingston et al., 2021). Additionally, a combination cholinergic treatment (central agonist and peripheral antagonist at muscarinic receptors, xanomeline and trospium respectively) demonstrated improved cognitive performance in SZ subjects with cognitive impairments (Sauder et al., 2022). Finally, adjunctive treatment of SZ with EPO improved cognitive performance (Li et al., 2018). Taken together, these data provide evidence that neuronal origin miRNA from serum EVs can be leveraged to identify disease associated signaling pathways enriched with treatment targets and is particularly relevant given that cognitive deficits in SZ are a strong predictor of poor outcomes and are not adequately treated.

The present inventors observed several miRNA consistently dysregulated across groups, the most prominent being hsa-miR-1246 with increased expression in all groups. Interestingly, the magnitude of miR-1246 differential expression increases as we move from SZ as a whole undifferentiated group, towards more focused comparisons with severe disease (CD and TRS), suggesting miR-1246 may be important to core features of schizophrenia and even more so to distinct biological domains within the diagnostic category. Furthermore, the present inventors found miR-1246 targets are specifically enriched in the brain and overlap with miR-137 targets. There is strong evidence for association between miR-137 and schizophrenia, via risk enhancing genetic variants in the host gene (MIR137), enrichment of genetic variants within validated targets of miR-137 and plausible pathways in which these targets function (Ripke et al., 2013; Kwon et al., 2013; Wright et al., 2015; Thomas et al., 2018). Given the overlap in miR-1246 and miR-137 targets, it is possible their combined regulation is important for sculpting gene expression in pathways and networks important for neuronal function. On the other hand, hsa-miR-451a was consistently reduced in CD and this may indicate specificity to severe cognitive impairments for a subset of subjects with schizophrenia. Interestingly, miR-451a was reduced in CSF exosomes from young (and late) onset Alzheimer's Disease subjects, suggesting miR-451a may report on cognitive decline in neurological disorders (Mckeever et al., 2018).

### Example 2 Neuronal Origin miRNA from Serum Extracellular Vesicles Associated with Schizophrenia are Conserved

In humans, the present inventors have identified neuronal origin miRNA from serum extracellular vesicles (EV's) that are associated with mental disorders (schizophrenia and schizophrenia subtypes). Animals, particularly non-human primates and mammals, also suffer from diseases that affect the central nervous system (Murray and Mitchell, 2022) and are similarly in need of relatively non-invasive tests that can aid in diagnosis, prognosis and treatment response. Additionally, livestock, companion and zoo (particularly endangered species) animal stress testing may be applicable given that the stress response in humans and rodents is mediated by miRNA (Hollins and Cairns, 2016) and this may allow opportunity to modify animal wellbeing, leading to improved meat quality in the case of livestock and behaviour in the case of companion and zoo animals. Therefore, the miRNA profiles identified as associated with neurological disorders may have veterinary applications, especially where the identified miRNA are conserved across species.

The present inventors used microRNAviewer (Kiezun et al., 2012) and UCSC genome browser to identify evolutionary conservation for thirty-five human miRNA that were identified from differential expression analysis (Table 18). The present inventors found most miRNA are conserved from vertebrates to primates, suggesting these miRNA play critical roles in fundamental organismal processes, such as mediating the response to toxic levels of environmental contaminants (miR-126) (Wang et al., 2013). Furthermore, miRNA with restricted conservation (mammals and primates) suggest these molecules are important for more recently emerged behaviours such as anxiety (miR-451a, miR-5100) (Du et al., 2019).

Taken together, the present inventors have shown that immuno-fractionation of serum EV's to obtain a neuronal origin miRNA profile has utility beyond human health, as most of the identified miRNA are evolutionary conserved, extending the work to include veterinary applications.

TABLE 18
Evolutionary conservation of psychiatric associated human miRNA

| | | | Mammals | | |
|---|---|---|---|---|---|
| | | | and | | |
| Vertebrates, mammals and primates | | | vertebrates | Primates and mammals | |
| let-7d-3p | miR-16-5p | miR-21-5p | miR-1843 | miR-1246 | miR-423-3p |
| miR-10b-5p | miR-199a-5p | miR-23a-3p | miR-451a | miR-3178 | miR-423-5p |
| miR-126-3p | miR-203a-3p | miR-26a-5p | let-7a-5p | miR-5100 | miR-484 |
| miR-126-5p | miR-205-5p | miR-26b-5p | let-7f-5p | miR-185-5p | miR-486-5p |
| miR-146b-5p | miR-20a-5p | miR-425-5p | miR-19b-3p | miR-324-5p | miR-590 |
| | | miR-93-5p | miR-92a-3p | | |

Underlined miRNA are mammals only and bold miRNA are primates only.
Note:
miR-7704, miR-4521 and miR-12136 are annotated to humans only.

### Example 3 Estimating the Purity of Neuronal-Origin Serum Extracellular Vesicles by Immuno-Fractionation of Total Serum Extracellular Vesicles

Extracellular vesicles (EV's) are released by most cell types and can be found in several biofluids, including the peripheral circulation. The biogenesis of EV's occurs intracellularly and as such the vesicle characteristics reflect the cell of origin. For example, neuronal EV's are enriched in miRNA and express neuronal proteins (Goldie et al., 2014) on their membrane surface (Tanner et al., 2000). These observations suggest that capture of neuronal origin serum EV's, by virtue of neuronal membrane antigens, can be leveraged to interrogate neuronal miRNA which are important molecules in synaptic plasticity (Aksoy-Aksel, Zampa and Schratt, 2014) and psychiatric disorders (Beveridge et al., 2010; Santarelli et al., 2011). Here, the present inventors demonstrate that immuno-fractionation of serum EV's to enrich for neuronal origin generates a unique miRNA profile and this has potential for biomarker development for brain disorders.

### Materials and Methods

Whole blood was obtained from a singly healthy male volunteer, immediately processed to serum and stored at −80° C. Enrichment for neuronal origin EV's was performed by incubating serum (250 μL) with protein G magnetic beads, antibodies (1 μg anti-MAP1B) and TBS supplemented with protease inhibitors to a final volume of 500 μL. The sample was incubated overnight at 4° C. with end-on-end rotation. Beads were washed three times, placed on magnet and supernatant (MAP1B-depleted fraction) was collected, combined with one volume exosome binding buffer and transferred to exoRNeasy spin columns, as per the manufacturer's instructions (Qiagen) for RNA extraction. The bead-bound fraction, representing neuronal origin EV's, was eluted by vortexing in 100 mM glycine (pH 2.8). Neutral pH was restored by addition of one-tenth volume 1M Tris-HCl (pH 8.5) and made up to 100 μL with TBS. The sample was placed on the magnet and eluent (MAP1B enriched) transferred to a clean tube, mixed with one volume exosome binding buffer and transferred to exoRNeasy spin columns, as per the manufacturer's instructions (Qiagen) for RNA extraction.

Additionally, to validate anti-MAP1B, the present inventors tested whether pre-clearing serum affected RNA recovery. Specifically, serum was incubated with naked beads to allow non-specific binding of serum proteins to be retained on-bead and supernatant (pre-cleared serum) recovered. Then, fresh antibody-coupled magnetic beads were incubated with pre-cleared serum or serum that was not pre-cleared, followed by total RNA extraction and quantification by NanoDrop. Secondly, RNA recovery was compared with and without anti-MAP1B.

Small RNA libraries were prepared from total RNA extracted from neuronal serum EV's, MAP1B depleted serum EV's and total serum EV's, using NEBNext Multiplex small RNA Library prep set for Illumina (New England BioLabs) and ran for 75 cycles of single end sequencing on Illumina NextSeq500. Relative abundance of each miRNA per fraction was calculated as miRNA count/total miRNA counts. The top one-third miRNA from neuronal enriched and neuronal depleted fractions, corresponding to relative abundance of >0.0127, were selected for further analysis.

### Results

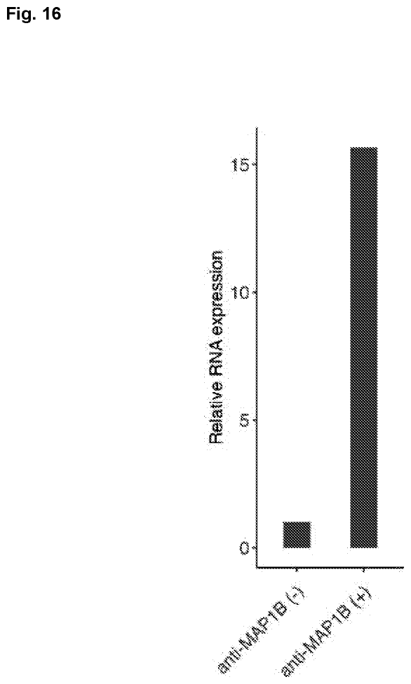

Serum pre-clearing had no effect on the amount of RNA recovered, demonstrated by equivalent RNA recovery from immuno-fractionated serum (RNA from pre-cleared serum=7.9 ng/UL and RNA from serum that was not pre-cleared=8.0 ng/μL). Furthermore, immuno-fractionation with and without anti-MAP1B demonstrated >15-fold recovery of RNA with the use of anti-MAP1B compared to no antibody (FIG. 16).

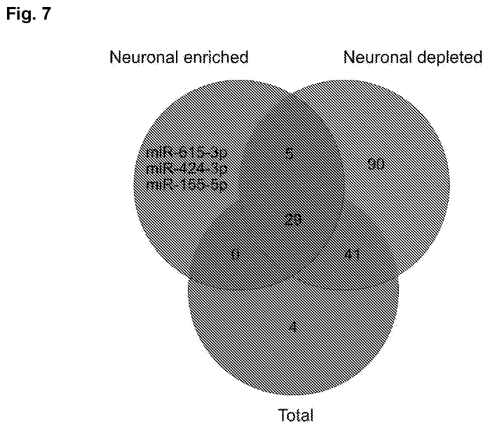

Small RNA sequencing of neuronal enriched, neuronal depleted and total serum EV's, identified 37, 165 and 74 miRNA in these fractions, respectively. Three miRNA were observed to be expressed only in neuronal enriched EV's; hsa-miR-615-3p, hsa-miR-424-3p and hsa-miR-155-5p (FIG. 7). These 3 miRNA are predicted to regulate the expression of synaptic genes involved in neuron development and associated with intellectual disability (Table 19).

TABLE 19
Neuronal origin miRNA from serum EV's target synaptic
genes associated with intellectual disability.

| | | | | | |
|---|---|---|---|---|---|
| Ontology ID | Ontology name | n | % | p-value | Bonferroni |

GO: Biological process

| | | | | | |
|---|---|---|---|---|---|
| GO: 0048666 | neuron development | 291 | 13.3 | 1.049E−20 | 1.091E−16 |
| GO: 0007417 | central nervous system | 303 | 13.8 | 1.604E−20 | 1.668E−16 |
| | development | | | | |
| GO: 1902680 | positive regulation of RNA | 320 | 14.6 | 3.544E−20 | 3.686E−16 |
| | biosynthetic process | | | | |
| GO: 1903508 | positive regulation of nucleic | 318 | 14.5 | 7.778E−20 | 8.091E−16 |
| | acid-templated transcription | | | | |
| GO: 0045893 | positive regulation of DNA- | 318 | 14.5 | 7.778E−20 | 8.091E−16 |
| | templated transcription | | | | |

GO: Cellular component

| | | | | | |
|---|---|---|---|---|---|
| GO: 000785 | chromatin | 229 | 10.5 | 1.546E−12 | 1.874E−9 |
| GO: 0045202 | synapse | 256 | 11.7 | 4.237E−11 | 5.135E−8 |
| GO: 0036477 | somatodendritic | 185 | 8.5 | 3.051E−8 | 3.698E−5 |
| | compartment | | | | |
| GO: 0098794 | postsynapse | 133 | 6.1 | 7.129E−8 | 8.641E−5 |
| GO: 0043025 | neuronal cell body | 133 | 6.1 | 1.926E−7 | 2.334E−4 |

GO: Disease

| | | | | | |
|---|---|---|---|---|---|
| C3714756 | intellectual disability | 228 | 10.4 | 3.118E−22 | 4.642E−18 |
| C1535926 | neurodevelopmental | 111 | 5.1 | 1.718E−17 | 2.558E−13 |
| | disorders | | | | |
| C0557874 | global developmental delay | 124 | 5.7 | 2.851E−15 | 4.245E−11 |
| C0424605 | developmental delay | 113 | 5.2 | 1.559E−14 | 2.321E−10 |
| | (disorder) | | | | |
| C0008073 | developmental disabilities | 73 | 3.3 | 3.875E−11 | 5.770E−7 |

n = number of annotated genes from input list, % = n/total number of genes from input list (total 2189).

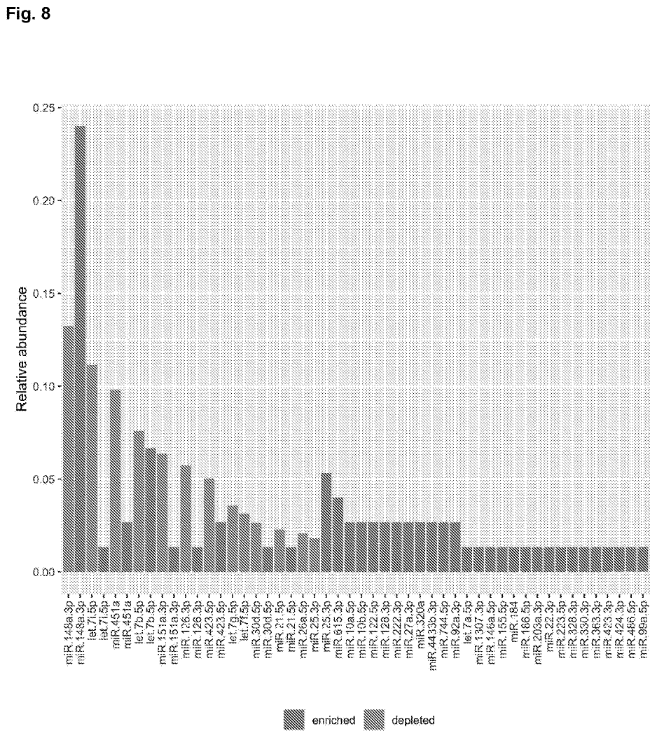

Given that neuronal origin EV's are a subset of total circulating EV's, the relative abundance of miRNA in neuronal origin enriched versus depleted fractions is likely to be distinct. Indeed, among the most abundant miRNA, relatively high expression of twenty-four miRNA in the neuronal enriched fraction was observed (FIG. 8). These data demonstrate that neuronal enrichment of serum EV's may be characterised by i) hsa-miR-615-3p, hsa-miR-424-3p and hsa-miR-155-5p only in the neuronal enriched fraction and/or ii) twenty-four miRNA in the neuronal enriched fraction at relative abundance of 0.013-0.027 (Table 20).

TABLE 20
Relative abundance of miRNA from neuronal enriched and depleted serum
EV's.

| | Relative abundance | | | Relative abundance | | | Relative abundance | |
|---|---|---|---|---|---|---|---|---|
| miRNA | enriched | depleted | miRNA | enriched | depleted | miRNA | enriched | depleted |
| miR-615- | 0.040 | nd | miR- | 0.013 | 0.011 | miR-328- | 0.013 | 0.001 |
| 3p | | | 146a-5p | | | 3p | | |
| (SEQ ID | | | (SEQ ID | | | (SEQ ID | | |
| NO: 30) | | | NO: 37) | | | NO: 45) | | |
| miR-155- | 0.013 | nd | miR-184 | 0.013 | <0.001 | miR-330- | 0.013 | <0.001 |
| 5p | | | (SEQ ID | | | 3p | | |
| (SEQ ID | | | NO: 38) | | | (SEQ ID | | |
| NO: 31) | | | | | | NO: 46) | | |
| miR-424- | 0.013 | nd | miR-186- | 0.013 | 0.003 | miR-363- | 0.013 | <0.001 |
| 3p | | | 5p | | | 3p | | |
| (SEQ ID | | | (SEQ ID | | | (SEQ ID | | |
| NO: 32) | | | NO: 39) | | | NO: 47) | | |
| let-7a-5p | 0.013 | 0.012 | miR- | 0.013 | <0.001 | miR-423- | 0.013 | 0.004 |
| (SEQ ID | | | 203a-3p | | | 3p | | |
| NO: 29) | | | (SEQ ID | | | (SEQ ID | | |
| | | | NO: 6) | | | NO: 48) | | |
| miR-10a- | 0.027 | 0.003 | miR-22- | 0.013 | 0.003 | miR- | 0.027 | 0.004 |
| 5p | | | 3p | | | 4433b-3p | | |
| (SEQ ID | | | (SEQ ID | | | (SEQ ID | | |
| NO: 33) | | | NO: 40) | | | NO: 49) | | |
| miR-10b- | 0.027 | 0.005 | miR-222- | 0.027 | 0.004 | miR-486- | 0.013 | 0.012 |
| 5p | | | 3p | | | 5p | | |
| (SEQ ID | | | (SEQ ID | | | (SEQ ID | | |
| NO: 21) | | | NO: 41) | | | NO: 2) | | |
| miR-122- | 0.027 | 0.004 | miR-223- | 0.013 | 0.003 | miR-744- | 0.027 | 0.005 |
| 5p | | | 5p | | | 5p | | |
| (SEQ ID | | | (SEQ ID | | | (SEQ ID | | |
| NO: 34) | | | NO: 42) | | | NO: 50) | | |
| miR-128- | 0.027 | <0.001 | miR-27a- | 0.027 | 0.003 | miR-92a- | 0.027 | 0.006 |
| 3p | | | 3p | | | 3p | | |
| (SEQ ID | | | (SEQ ID | | | (SEQ ID | | |
| NO: 35) | | | NO: 43) | | | NO: 9) | | |
| miR-1307- | 0.013 | <0.001 | miR-320a- | 0.027 | 0.005 | miR-99a- | 0.013 | 0.005 |
| 3p | | | 3p | | | 5p | | |
| (SEQ ID | | | (SEQ ID | | | (SEQ ID | | |
| NO: 36) | | | NO: 44) | | | NO: 51) | | |

### Discussion

The present inventors have demonstrated that the use of anti-MAP1B coupled to magnetic beads to immuno-fractionate serum EV's, is fit for the purpose of enrichment for neuronal origin EVs from human serum. As there was equivalent RNA recovered from serum and pre-cleared serum, this suggests that non-specific binding of serum proteins to the magnetic beads either does not occur or has no measurable effect on neuronal EV enrichment (determined by RNA quantification), whereas the presence of anti-MAP1B had a very large effect on RNA recovery. These two observations allow for reduced sample handling (as there is no need for pre-clearing) and therefore reduced variation due to technical factors while also maximizing the number of samples that can be processed with the described protocol and maximizing the quantity of RNA recovered.

Furthermore, the described enrichment protocol results in a miRNA population that is distinct from the total and remaining vesicle-associated population in the circulation. Importantly, three miRNA (hsa-miR-615-3p, hsa-miR-424-3p and hsa-miR-155-5p) were observed only in the neuronal fraction and another twenty-four miRNA were observed in the top third highest abundance for neuronal enriched but not neuronal depleted. This suggests that the enrichment protocol may have application to identification of molecular biomarkers for disorders that affect neuronal cells. The human brain is composed of billions of neurons but is largely inaccessible in living subjects, especially for molecular studies. Therefore, the ability to access state biomarkers associated with brain disorders from a relatively non-invasive sampling of the peripheral circulation represents an important advance for diagnosis, prognosis and treatment response of many conditions including psychiatric, neurodegenerative and traumatic injury.

TABLE 21
miRNA from neuronal enriched serum EV's.

Seed sequence

>hsa-miR-615-3p MIMAT0003283

| | |
|---|---|
| UCCGAGCCUGGGUCUCCCUCUU | CCGAGCC |
| (SEQ ID NO: 30) | (SEQ ID NO: 63) |

>hsa-miR-155-5p MIMAT0000646

| | |
|---|---|
| UUAAUGCUAAUCGUGAUAGGGGUU | UAAUGCU |
| (SEQ ID NO: 31) | (SEQ ID NO: 64) |

>hsa-miR-424-3p MIMAT0004749

| | |
|---|---|
| CAAAACGUGAGGCGCUGCUAU | AAAACGU |
| (SEQ ID NO: 32) | (SEQ ID NO: 65) |

The claims defining the invention are as follows:

1. A method of diagnosing a disease state of a subject, said method comprising the steps of:
a) measuring the level of one or more small non-coding RNA in a sample from the subject, wherein the one or more small non-coding RNA comprises a nucleotide sequence shown in any one of SEQ ID NOs: 1 to 29, and wherein, the sample comprises extracellular vesicles; and
b) comparing the level of the one or more small non-coding RNA to a reference level of the one or more small non-coding RNA, wherein an increased or decreased level of the one or more small non-coding RNA compared to the reference level is indicative of a disease state or a stage of development of a disease state.

2. The method according to claim 1, wherein the reference level of the one or more small non-coding RNA is the level of the one or more small non-coding RNA in a sample from a normal subject.

3. The method according to claim 1 or claim 2, wherein the reference level of the one or more small non-coding RNA is a threshold level.

4. The method according to claim 3, wherein the threshold level is a cut-off value.

5. The method according to any one of claims 1 to 4, wherein the one or more small non-coding RNA is a microRNA (miRNA).

6. The method according to any one of claims 1 to 5, wherein extracellular vesicles in the sample comprise microvesicles and/or exosomes.

7. The method according to any one of claims 1 to 6, wherein the sample comprising extracellular vesicles is a sample enriched for extracellular vesicles.

8. The method according to any one of claims 1 to 7, wherein the sample is blood, serum or plasma.

9. The method according to any one of claims 1 to 8, wherein the extracellular vesicles are exosomes.

10. The method according to any one of claims 1 to 9, wherein the disease is a neurodegenerative disease or neuropsychiatric disease.

11. The method according to any one of claims 1 to 10, wherein the disease is Schizophrenia.

12. The method according to claim 11, wherein an increased level of a small non-coding RNA comprising the nucleotide sequence shown in SEQ ID NO: 1 compared to the reference level of the small non-coding RNA is indicative of Schizophrenia or a predisposition to Schizophrenia.

13. The method according to claim 12, wherein a log 2 fold increase of at least about 0.8 compared to a reference level of the small non-coding RNA in a sample from a normal subject is indicative of Schizophrenia or a predisposition to Schizophrenia.

14. The method according to any one of claims 11 to 13, wherein a decreased level of a small non-coding RNA comprising the nucleotide sequence shown in SEQ ID NO: 2 compared to the reference level of the small non-coding RNA is indicative of Schizophrenia or a predisposition to Schizophrenia.

15. The method according to claim 14, wherein a log₂ fold decrease of at least about 0.5 compared to a reference level of the small non-coding RNA in a sample from a normal subject is indicative of Schizophrenia or a predisposition to Schizophrenia.

16. The method of any one of claims 1 to 10, wherein the disease is Schizophrenia with cognitive deficit.

17. The method according to claim 16, wherein an increased level of one or more small non-coding RNA(s) comprising the nucleotide sequence shown in any one of SEQ ID NOs: 1, and 3 to 7 compared to the reference level(s) of the one or more small non-coding RNA(s) is indicative of Schizophrenia with cognitive deficit.

18. The method according to claim 17, wherein a log₂ fold increase of at least about 0.7 to at least about 1.3 compared to a reference level(s) of the one or more small non-coding RNA(s) in a sample from a normal subject is indicative of Schizophrenia with cognitive deficit.

19. The method according to any one of claims 16 to 18, wherein an increased level of a small non-coding RNA comprising the nucleotide sequence shown in SEQ ID NO: 1 compared to the reference level of the non-coding RNA is indicative of Schizophrenia with cognitive deficit.

20. The method according to claim 19, wherein a log₂ fold increase of at least about 1.3 compared to a reference level of the small non-coding RNA in a sample from a normal subject is indicative of Schizophrenia with cognitive deficit.

21. The method according to any one of claims 16 to 20, wherein an increased level of one or more small non-coding RNA(s) comprising the nucleotide sequence shown in any one of SEQ ID NOs: 3, 5, and 7 compared to the reference level(s) of the one or more small non-coding RNA(s) is indicative of Schizophrenia with cognitive deficit.

22. The method according to claim 21, wherein a log₂ fold increase of at least about 0.7 to at least about 1.1 compared to a reference level(s) of the one or more small non-coding RNA(s) in a sample from a normal subject is indicative of Schizophrenia with cognitive deficit.

23. The method according to any one of claims 16 to 22, wherein an increased level of one or more small non-coding RNA(s) comprising the nucleotide sequence shown in any one of SEQ ID NOs: 4 and 6 compared to the reference level(s) of the one or more small non-coding RNA(s) is indicative of Schizophrenia with cognitive deficit.

24. The method according to claim 23, wherein a log₂ fold increase of at least about 0.8 to at least about 0.9 compared to a reference level(s) of the one or more small non-coding RNA(s) in a sample from a normal subject is indicative of Schizophrenia with cognitive deficit.

25. The method according to any one of claims 16 to 24, wherein a decreased level of one or more small non-coding RNA(s) comprising the nucleotide sequence shown in any one of SEQ ID NOs: 2, and 8 to 11 compared to the reference level(s) of the one or more small non-coding RNA(s) is indicative of Schizophrenia with cognitive deficit.

26. The method according to claim 25, wherein a log₂ fold decrease of at least about 0.4 to at least about 1.3 compared to a reference level(s) of the one or more small non-coding RNA(s) in a sample from a normal subject is indicative of Schizophrenia with cognitive deficit.

27. The method according to any one of claims 16 to 26, wherein a decreased level of a small non-coding RNA comprising the nucleotide sequence shown in SEQ ID NO: 8 compared to the reference level of the small non-coding RNA is indicative of Schizophrenia with cognitive deficit.

28. The method according to claim 27, wherein a log₂ fold decrease of at least about 1.3 compared to a reference level of the one or more small non-coding RNA in a sample from a normal subject is indicative of Schizophrenia with cognitive deficit.

29. The method according to any one of claims 16 to 28, wherein a decreased level of a small non-coding RNA comprising the nucleotide sequence shown in SEQ ID NO: 2 compared to the reference level of the small non-coding RNA is indicative of Schizophrenia with cognitive deficit.

30. The method according to claim 29, wherein a log₂ fold decrease of at least about 0.7 compared to a reference level of the one or more small non-coding RNA in a sample from a normal subject is indicative of Schizophrenia with cognitive deficit.

31. The method according to claim 16, wherein an increased level of one or more small non-coding RNA(s) comprising the nucleotide sequence shown in any one of SEQ ID NOs: 1, 3, 5, and 7 compared to the reference level(s) of the one or more small non-coding RNA(s) is indicative of Schizophrenia with cognitive deficit.

32. The method according to claim 31, wherein a log₂ fold increase of at least about 1.0 to at least about 1.3 compared to a reference level(s) of the one or more small non-coding RNA(s) in a sample from a subject with cognitively spared Schizophrenia is indicative of Schizophrenia with cognitive deficit.

33. The method according to any one of claim 16, 31, or 32, wherein an increased level of a small non-coding RNA comprising the nucleotide sequence shown in SEQ ID NO: 1 compared to the reference level of the small non-coding RNA is indicative of Schizophrenia with cognitive deficit.

34. The method according to claim 33, wherein a log₂ fold increase of at least about 1 compared to a reference level of the small non-coding RNA in a sample from a subject with cognitively spared Schizophrenia is indicative of Schizophrenia with cognitive deficit.

35. The method according to any one of claims 16, or 31 to 34, wherein an increased level of one or more small non-coding RNA(s) comprising the nucleotide sequence shown in any one of SEQ ID NOs: 3, 5, and 7 compared to the reference level(s) of the one or more small non-coding RNA(s) is indicative of Schizophrenia with cognitive deficit.

36. The method according to claim 35, wherein a log₂ fold increase of at least about 1 to at least about 1.3 compared to a reference level(s) of the one or more small non-coding RNA(s) in a sample from a subject with cognitively spared Schizophrenia is indicative of Schizophrenia with cognitive deficit.

37. The method according to any one of claims 16, or 31 to 36, wherein a decreased level of a small non-coding RNA comprising the nucleotide sequence shown in SEQ ID NO: 8 compared to the reference level of the one or more small non-coding RNA is indicative of Schizophrenia with cognitive deficit.

38. The method according to claim 37, wherein a log₂ fold decrease of at least about 1.2 compared to a reference level of the small non-coding RNA in a sample from a subject with cognitively spared Schizophrenia is indicative of Schizophrenia with cognitive deficit.

39. The method of any one of claims 1 to 10, wherein the disease is cognitively spared Schizophrenia.

40. The method according to claim 39, wherein a decreased level of at least one or more small non-coding RNA(s) comprising the nucleotide sequence shown in any one of SEQ ID NOs: 2, and 12 to 17 compared to the reference level(s) of the one or more small non-coding RNA(s) is indicative of cognitively spared Schizophrenia.

41. The method according to claim 40, wherein a log₂ fold decrease of at least about 0.4 to at least about 0.8 compared to a reference level(s) of the small non-coding RNA(s) in a sample from a normal subject is indicative of cognitively spared Schizophrenia.

42. The method of any one of claims 1 to 10, wherein the disease is treatment resistant Schizophrenia.

43. The method according to claim 42, wherein an increased level of at least one or more small non-coding RNA(s) comprising the nucleotide sequence shown in any one of SEQ ID NOs: 1, and 3 to 7 compared to the reference level(s) of the one or more small non-coding RNA(s) is indicative of treatment resistant Schizophrenia.

44. The method according to claim 43, wherein a log₂ fold increase of at least about 1.1 to at least about 2.6 compared to a reference level(s) of the small non-coding RNA(s) in a sample from a subject with Schizophrenia that is not treatment resistant is indicative of treatment resistant Schizophrenia.

45. The method according to any one of claims 42 to 44, wherein an increased level of a small non-coding RNA comprising the nucleotide sequence shown in SEQ ID NO: 1 compared to the reference level of the small non-coding RNA is indicative of treatment resistant Schizophrenia.

46. The method according to claim 45, wherein a log₂ fold increase of at least about 2 compared to a reference level of the small non-coding RNA in a sample from a subject with Schizophrenia that is not treatment resistant is indicative of treatment resistant Schizophrenia.

47. The method according to any one of claims 42 to 46, wherein an increased level of one or more small non-coding RNA(s) comprising the nucleotide sequence shown in any one of SEQ ID NOs: 3, 5, and 7 compared to the reference level(s) of the one or more small non-coding RNA(s) is indicative of treatment resistant Schizophrenia.

48. The method according to claim 47, wherein a log₂ fold increase of at least about 2.3 to at least about 2.6 compared to a reference level(s) of the one or more small non-coding RNA(s) in a sample from a subject with Schizophrenia that is not treatment resistant is indicative of treatment resistant Schizophrenia.

49. The method according to any one of claims 42 to 48, wherein an increased level of at least one or more small non-coding RNA(s) comprising the nucleotide sequence shown in any one of SEQ ID NOs: 4 and 6 compared to the reference level(s) of the one or more small non-coding RNA(s) is indicative of treatment resistant Schizophrenia.

50. The method according to claim 49, wherein a log₂ fold increase of at least about 1.1 to at least about 1.3 compared to a reference level(s) of the small non-coding RNA in a sample from a subject with Schizophrenia that is not treatment resistant is indicative of treatment resistant Schizophrenia.

51. The method according to any one of claims 42 to 50, wherein a decreased level of at least one or more small non-coding RNA(s) comprising the nucleotide sequence shown in any one of SEQ ID NOs: 18 to 20 compared to the reference level(s) of the one or more small non-coding RNA(s) is indicative of treatment resistant Schizophrenia.

52. The method according to claim 51, wherein the small non-coding RNA comprises the nucleotide sequence shown in SEQ ID NO: 18.

53. The method according to claim 52, wherein a log₂ fold decrease of at least about 1.4 compared to a reference level(s) of the one or more small non-coding RNA in a sample from a subject with Schizophrenia that is not treatment resistant, or from a pooled sample from a subject with Schizophrenia that is not treatment resistant and a normal subject is indicative of treatment resistant Schizophrenia.

54. The method of any one of claims 1 to 10, wherein the disease is Schizophrenia with depression.

55. The method according to claim 54, wherein an increased level of a small non-coding RNA comprising the nucleotide sequence shown in SEQ ID NO: 4 compared to the reference level(s) of the small non-coding RNA is indicative of Schizophrenia with depression.

56. The method according to claim 55, wherein a log₂ fold increase of at least about 1.1 compared to a reference level of the small non-coding RNA in a sample from a non-depressed subject with Schizophrenia or from a normal subject, or from a pooled sample from a non-depressed subject with Schizophrenia and a normal subject is indicative of Schizophrenia with depression.

57. The method according to claim any one of claims 1 to 56, wherein the method further comprises measuring the total circulating neuronal origin extracellular vesicle small non-coding RNA level.

58. The method according to claim 57, wherein a log₁₀ fold decrease of at least about 0.3 total circulating neuronal origin extracellular vesicle small non-coding RNA compared to a reference level of total circulating neuronal origin extracellular vesicle small non-coding RNA in a sample from a normal subject is indicative of Schizophrenia or a predisposition to Schizophrenia.

59. The method according to claim 57, wherein a log₁₀ fold decrease of at least about 0.3 total circulating neuronal origin extracellular vesicle small non-coding RNA compared to a reference level of total circulating neuronal origin extracellular vesicle small non-coding RNA in a sample from a normal subject is indicative of Schizophrenia with cognitive deficit.

60. The method according to claim 57, wherein a log₁₀₂ fold decrease of at least about 0.3 total circulating neuronal origin extracellular vesicle small non-coding RNA compared to a reference level of total circulating neuronal origin extracellular vesicle small non-coding RNA in a sample from a normal subject is indicative of Schizophrenia with spared cognition.

61. The method of any one of claims 1 to 60, further comprising a psychological, behavioural, physiological and/or genetic assessment of the subject.

62. The method of claim 61, wherein the psychological assessment determines the presence and/or level of cognitive impairment.

63. The method of any one of claims 1 to 62, further comprising selecting a treatment or modifying a treatment for the disease state or the predisposition to the disease state, based on the diagnosis of disease state.

64. The method of any one of claims 1 to 63, further comprising administering to the subject a therapeutically effective amount of an anti-disease state therapeutic.

65. A method for preparing an extracellular vesicle fraction enriched for neuron derived extracellular vesicles from a sample for analysing small non-coding RNA involved in a disease state, comprising:
(a) providing a biological sample;
(b) contacting the sample with a microtubule associated protein (MAP) binding agent to selectively bind extracellular vesicles expressing MAP; and
(c) isolating the bound extracellular vesicles.

66. The method according to claim 65, wherein the method further comprises:
(d) extracting RNA from the extracellular vesicles; and
(e) analysing the level of the one or more small non-coding RNA to a reference level of the one or more small non-coding RNA, wherein an increased or decreased level of the one or more small non-coding RNA compared to the reference level is indicative of a disease state or a stage of development of a disease state.

67. The method according to claim 65 or claim 66, wherein the MAP is MAP1B.

68. The method according to any one of claims 65 to 67, wherein the MAP binding agent is immobilised to a non-porous support.

69. The method according to any one of claims 65 to 68, wherein isolating the bound extracellular vesicles comprises:
(i) removing the unbound fraction
(ii) releasing the extracellular vesicles from the binding agent.

70. The method according to any one of claims 65 to 69, wherein the MAP binding agent is an antibody or binding fragment thereof.

71. A method for the isolation of neuron derived extracellular from a blood sample, comprising:
(a) contacting the sample with a microtubule associated protein (MAP) binding agent to selectively bind extracellular vesicles expressing MAP1B; and
(b) isolating the bound extracellular vesicles.

72. A population of neuron derived extracellular vesicles, wherein the extracellular vesicles comprise one or more small non-coding RNA comprising a sequence selected from any one of SEQ ID NOs: 2, 6, 9, 21, or 29 to 51.

73. A population of neuron derived extracellular vesicles, wherein the extracellular vesicles comprise one or more small non-coding RNA comprising a sequence selected from any one of SEQ ID NOs: 30 to 32.

74. The population of claim 73, wherein the extracellular vesicles comprise one or more additional small non-coding RNA comprising a sequence selected from any one of SEQ ID NOs: 2, 6, 9, 21, 29, or 33 to 51.

75. The population of any one of claims 72 to 74, wherein the relative abundance of the one or more small non-coding RNA is about 0.013 to about 0.027.

76. The population according to any one of claims 72 to 75, wherein the extracellular vesicles are isolated according to the method of claim 71.

77. The method of any one of claims 1 to 64, wherein the method further comprises preparing an extracellular vesicle fraction enriched for neuron derived extracellular vesicles from the sample according to the method of any one of claims 65 to 70 for analysing small non-coding RNA(s) involved in a disease state.

78. A kit for the isolation of neuron derived extracellular vesicles from a blood sample comprising:
(a) a microtubule associated protein (MAP) binding agent; and
(b) optionally, one or more agents selected a solid support, wash buffer, elution buffer.
